(19) United States
(12) Patent Application Publication
(10) Pub. No.: US 2024/0336559 A1
(43) Pub. Date: Oct. 10, 2024

(54) ANTI-FUNGALS COMPOUNDS TARGETING THE SYNTHESIS OF FUNGAL SPHINGOLIPIDS

(71) Applicant: The Research Foundation for the State University of New York, Albany, NY (US)

(72) Inventors: Iwao Ojima, Stony Brook, NY (US); Krupanandan HARANAHALLI, Stony Brook, NY (US); Maurizio DEL POETA, Stony Brook, NY (US); Ashna GARG, Stony Brook, NY (US)

(73) Assignee: The Research Foundation for the State University of New York, Albany, NY (US)

(21) Appl. No.: 18/698,584

(22) PCT Filed: Oct. 6, 2022
(86) PCT No.: PCT/US2022/077653

§ 371 Date: Apr. 4, 2024

Related U.S. Application Data
(60) Provisional application No. 63/252,795, filed on Oct. 6, 2021.

(51) Int. Cl. C07C 251/86; A61K 31/166; A61K 31/396; A61P 31/10; C07D 229/02

(52) U.S. Cl. CPC C07C 251/86; A61K 31/166; A61K 31/396; A61P 31/10; C07D 229/02

(57) ABSTRACT

The present invention provides a compound having the structure:

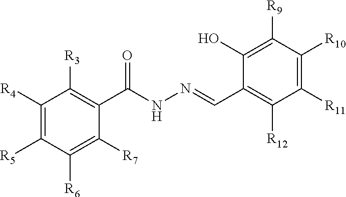

wherein
R₃, R₄, R₅, R₆, and R₇ are each independently, H, halogen, CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, CN, alkenyl, alkynyl, aryl, heteroaryl, cycloalkyl, heterocycloalkyl, —OAc, —COR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl,
   wherein each occurrence of R₁₄ is independently —H, alkyl, alkenyl, alkynyl, aryl, or heteroaryl,
   wherein each occurrence of R₁₅ is independently —H, alkyl, alkenyl, alkynyl, aryl, or heteroaryl,
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H.

This application claims the benefit of U.S. Provisional Application No. 63/252,795, filed Oct. 6, 2021, the content of which is hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### GOVERNMENT SUPPORT

This invention was made with government support under AI 116420 awarded by the National Institutes of Health. The government has certain rights in the invention.

### BACKGROUND OF THE INVENTION

According to recent statistics, nearly 300 million people are affected by serious fungal infections globally (Fungal Infection Trust 2011). Current anti-fungal agents possessing serious drawbacks such as drug-drug interactions, toxicity and narrow spectrum of activity and with an increase in the emergence of resistant strains of fungi to these drugs, there is a desperate need for the development of new antifungal agents with novel mechanisms of action (Mor V, Rella, 2015, Lazzarini C, 2018).

A particular challenge with the discovery of antifungal drugs is toxicity due to the similarities between the fungal and human eukaryotic genomes. Recent studies have focused on the role of bioactive lipids in fungal pathogenesis. In exploring potential therapeutic targets, it became apparent that fungal sphingolipid pathways are quite distinct from human sphingolipid pathways. In addition, the sphingolipid pathway is involved in the virulence of clinically important pathogenic fungi including Cryptococcus neoformans (Cn). The fungal sphingolipid complex, glucosylceramide (GlcCer), has increased expression on the fungal membrane in a lung infection model. GlcCer maintains fungal cell membrane integrity and represents an attractive therapeutic target. In addition, gene deletion of glucosylceramide synthase (Gcs1) results in the creation of a C. neoformans strain, Δgcs1, that does not cause morbidity or mortality in a mouse model of CM. Moreover, Δgcs1 fungi exhibit deficient growth in vitro at a pH of >7, a similar pH to that found in the extracellular alveolar space in the lung where Cn thrives and is the predominant first site of infection (Talar B. Kechichian, 2007). Because of these preliminary data, a chemical library of 49,120 compounds from ChemBridge was screened to detect inhibition of fungal growth at alkaline pH (>7). Several of the early leads exhibited dose dependent inhibition of GlcCer synthesis in C. neoformans but no effect on mammalian GlcCer synthesis.

Based on these preliminary results, a library of novel arylhydrazone compositions were designed and synthesized and evaluated for their potency. The SAR study indicated that a 2-hydroxyl group in the aromatic B-ring of the acylhydrazones (FIG. 1) is preferred for antifungal activity. The compositions described herein provide analogs with substituted aromatic/heteroaromatic groups in the ring B, especially with ethynyl and alkynyl groups as substituents. Their antifungal activities were examined, and a number of compounds exhibited comparable potencies and favorable pharmacological properties.

C. neoformans (Cn) is an opportunistic pathogen and a major cause of fungal disease, especially in immunocompromised patients including HIV positive individuals, transplant patients, patients with reticuloendothelial malignancies, and those being treated with corticosteroids. More recently, Cryptococcus gattii has emerged as an important pathogen that is able to infect healthy individuals. Following initial infection in the lung, Cn and C. gattii commonly spread to most major organ systems including the central nervous system. The first-line therapeutic remains Amphotericin B (AmB), often co-administered with flucytosine, followed by ongoing maintenance therapy with fluconazole. Even in newer liposomal formulations, AmB side effects may include hypokalemia, headache, nausea, vomiting, nephrotoxicity, and cardiac failure. Moreover, echinocandins do not show any activity against Cryptococcus in vivo, and newer triazoles including voriconazole have not shown comparable efficacy to AmB in clinical studies. Moreover, for patients who fail to respond to AmB or who are too ill to receive it, there is a lack of effective, well-tolerated alternatives. Although other factors contribute to the high mortality associated with CM, failure of CM patients to respond to AmB severely reduces their chances of survival and alternative therapeutics are desperately needed.

The compositions and methods described herein have broad spectrum of antifungal activity, little toxicity in vitro and in animal models. The compositions and methods have a different mechanism of action and thus are synergistic with current antifungals, and do not develop resistance, at least in vitro.

These new antifungal agents (NCEs) can be used not only to treat humans affected by fungi but also to treat animals and plants affected by fungal organisms. Thus, these NCEs will have humans as well as veterinarian and agricultural use.

### SUMMARY OF THE INVENTION

The present invention provides a compound having the structure:

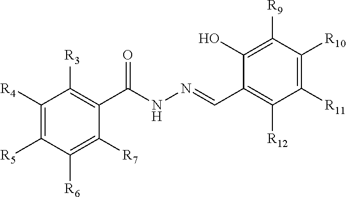

wherein
R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, —CN, alkenyl, alkynyl, aryl, heteroaryl, cycloalkyl, heterocycloalkyl, —OAc, —OR₁₃, —COR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
wherein when one of R₉, R₁₀, R₁₁, and R₁₂ is —OR₁₃, R₃, R₄, R₅, R₆, and R₇ are not halogen or alkyl.

The present invention provides a compound having the structure:

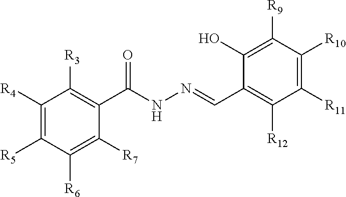

wherein
   R₃, R₄, R₅, R₆, and R₇ are each independently H, alkenyl, alkynyl, aryl, heteroaryl, —OAc, —COR₁₃, —CH₂OR₁₃, —SH, —SR₁₃, —SO₂R₁₃, —NH₂, —NR₁₄R₁₅, —NHCOR₁₂, or —CONR₁₄R₁₅;
   R₉, R₁₀, R₁₁, R₁₂ are each independently —H, halogen, —CN, —CF₃, —OCF₃, —NO₂, alkyl, alkenyl, alkynyl, aryl, heteroaryl, —OH, —OAc, —OR₁₃, —COR₁₃, —SH, —SR₁₃, —SO₂R₁₃, —SO₂NR₁₄R₁₅, —NH₂, —NHR₁₃, NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is H, R₁₅ is not methyl or when R₁₅ is H, R₁₄ is not methyl;
wherein at least one of R₉, R₁₀, R₁₁, and R₁₂ is not H;
wherein at least one of R₃, R₄, R₅, R₆, and R₇ is not H.

The present invention provides a method of inhibiting the growth of a fungus comprising contacting the fungus with an effective amount of a compound having the structure:

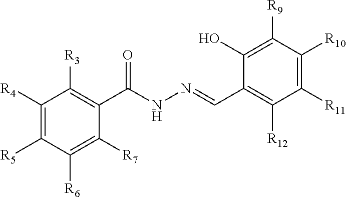

wherein
R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, —CN, alkenyl, alkynyl, aryl, heteroaryl, cycloalkyl, heterocycloalkyl, —OAc, —OR₁₃, —COR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
wherein when one of R₉, R₁₀, R₁₁, and R₁₂ is —OR₁₃, R₃, R₄, R₅, R₆, and R₇ are not halogen or alkyl;
or a pharmaceutically acceptable salt or ester thereof, so as to thereby inhibit the growth of the fungus.

The present invention provides method of inhibiting fungal sphingolipid synthesis in a fungus comprising contacting the fungus with an effective amount of a compound having the structure:

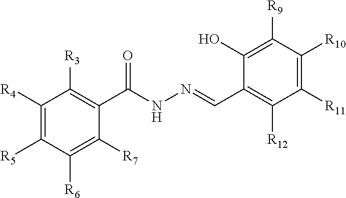

wherein
R₃, R₄, R₅, R₆ and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, —CN, alkenyl, alkynyl, aryl, heteroaryl, cycloalkyl, heterocycloalkyl, —OAc, —OR₁₃, —COR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
wherein when one of R₉, R₁₀, R₁₁, and R₁₂ is —OR₁₃, R₃, R₄, R₅, R₆, and R₇ are not halogen or alkyl;
or a pharmaceutically acceptable salt or ester thereof, so as to thereby inhibit sphingolipid synthesis in the fungus.

The present invention provides a method of inhibiting fungal sphingolipid synthesis in a fungus in a mammal without substantially inhibiting mammalian sphingolipid synthesis comprising administering to the mammal an effective amount of a compound having the structure:

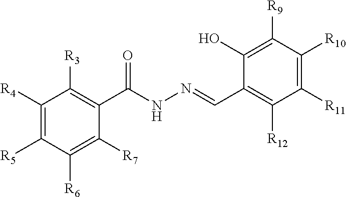

wherein
R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, —CN, alkenyl, alkynyl, aryl, heteroaryl, cycloalkyl, heterocycloalkyl, —OAc, —OR₁₃, —COR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
      wherein when R₁₄ is methyl, R₁₅ is not methyl;
      wherein at least one of R₉, R₁₀, R₁₁, and R₁₂ is not H;
      wherein at least one of R₃, R₄, R₅, R₆, and R₇ is not H;
      wherein when one of R₉, R₁₀, R₁₁, and R₁₂ is —OR₁₃, R₃, R₄, R₅, R₆, and R₇ are not halogen or alkyl;
or a pharmaceutically acceptable salt or ester thereof, so as to thereby inhibit fungal sphingolipid synthesis in the fungus in the mammal without substantially inhibiting mammalian sphingolipid synthesis.

The present invention provides a method of inhibiting the growth of a fungus comprising contacting the fungus with an effective amount of a compound having the structure:

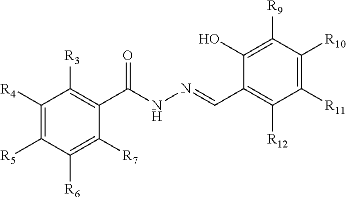

wherein
   R₃, R₄, R₅, R₆, and R₇ are each independently H, alkenyl, alkynyl, aryl, heteroaryl, —OAc, —COR₁₃, —CH₂OR₁₃, —SH, —SR₁₃, —SO₂R₁₃, —NH₂, —NR₁₄R₁₅, —NHCOR₁₂, or —CONR₁₄R₁₅;
   R₉, R₁₀, R₁₁, R₁₂ are each independently —H, halogen, —CN, —CF₃, —OCF₃, —NO₂, alkyl, alkenyl, alkynyl, aryl, heteroaryl, —OH, —OAc, —OR₁₃, —COR₁₃, —SH, —SR₁₃, —SO₂R₁₃, —SO₂NR₁₄R₁₅, —NH₂, —NHR₁₃, NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is H, R₁₅ is not methyl or when R₁₅ is H, R₁₄ is not methyl;
wherein at least one of R₉, R₁₀, R₁₁, and R₁₂ is not H;
wherein at least one of R₃, R₄, R₅, R₆, and R₇ is not H;
or a pharmaceutically acceptable salt or ester thereof, so as to thereby inhibit the growth of the fungus.

The present invention provides a method of inhibiting fungal sphingolipid synthesis in a fungus comprising contacting the fungus with an effective amount of a compound having the structure:

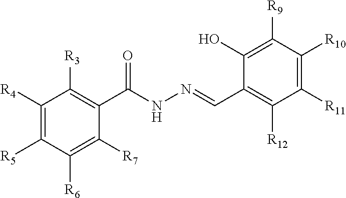

wherein
R₃, R₄, R₅, R₆, and R₇ are each independently H, alkenyl, alkynyl, aryl, heteroaryl, —OAc, —COR₁₃, —CH₂OR₁₃, —SH, —SR₁₃, —SO₂R₁₃, —NH₂, —NR₁₄R₁₅, —NHCOR₁₂, or —CONR₁₄R₁₅;
R₉, R₁₀, R₁₁, R₁₂ are each independently —H, halogen, —CN, —CF₃, —OCF₃, —NO₂, alkyl, alkenyl, alkynyl, aryl, heteroaryl, —OH, —OAc, —OR₁₃, —COR₁₃, —SH, —SR₁₃, —SO₂R₁₃, —SO₂NR₁₄R₁₅, —NH₂, —NHR₁₃, NR₁₄R₁₅, —NHCOR₁₂, or —CONR₁₄R₁₅;
wherein each occurrence of R₁₃ is independently alkyl, alkenyl, alkynyl, aryl, or heteroaryl;
wherein each occurrence of R₁₄ is independently —H, alkyl, alkenyl, alkynyl, aryl, or heteroaryl;
wherein each occurrence of R₁₅ is independently —H, alkyl, alkenyl, alkynyl, aryl, or heteroaryl;
wherein when R₁₄ is H, R₁₅ is not methyl or when R₁₅ is H, R₁₄ is not methyl;
wherein at least one of R₉, R₁₀, R₁₁, and R₁₂ is not H;
wherein at least one of R₃, R₄, R₅, R₆, and R₇ is not H;
or a pharmaceutically acceptable salt or ester thereof, so as to thereby inhibit sphingolipid synthesis in the fungus.

The present invention provides a method of inhibiting fungal sphingolipid synthesis in a fungus in a mammal without substantially inhibiting mammalian sphingolipid synthesis comprising administering to the mammal an effective amount of a compound having the structure:

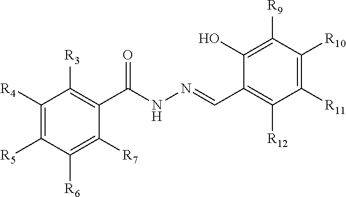

wherein
R₃, R₄, R₅, R₆, and R₇ are each independently H, alkenyl, alkynyl, aryl, heteroaryl, —OAc, —COR₁₃, —CH₂OR₁₃, —SH, —SR₁₃, —SO₂R₁₃, —NH₂, —NR₁₄R₁₅, —NHCOR₁₂, or —CONR₁₄R₁₅;
R₉, R₁₀, R₁₁, R₁₂ are each independently —H, halogen, —CN, —CF₃, —OCF₃, —NO₂, alkyl, alkenyl, alkynyl, aryl, heteroaryl, —OH, —OAc, —OR₁₃, —COR₁₃, —SH, —SR₁₃, —SO₂R₁₃, —SO₂NR₁₄R₁₅, —NH₂, —NHR₁₃, NR₁₄R₁₅, —NHCOR₁₂, or —CONR₁₄R₁₅;
wherein each occurrence of R₁₃ is independently alkyl, alkenyl, alkynyl, aryl, or heteroaryl;
wherein each occurrence of R₁₄ is independently —H, alkyl, alkenyl, alkynyl, aryl, or heteroaryl;
wherein each occurrence of R₁₅ is independently —H, alkyl, alkenyl, alkynyl, aryl, or heteroaryl;
wherein when R₁₄ is H, R₁₅ is not methyl or when R₁₅ is H, R₁₄ is not methyl;
wherein at least one of R₉, R₁₀, R₁₁, and R₁₂ is not H;
wherein at least one of R₃, R₄, R₅, R₆, and R₇ is not H;
or a pharmaceutically acceptable salt or ester thereof, so as to thereby inhibit fungal sphingolipid synthesis in the fungus in the mammal without substantially inhibiting mammalian sphingolipid synthesis.

### BRIEF DESCRIPTION OF THE FIGURES

FIG. 1. Structure of acylhydrazones.

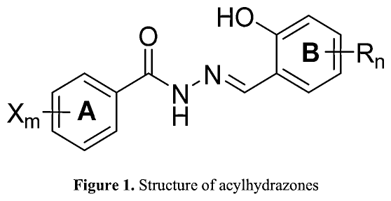

FIG. 2. Inhibition of GlcCer from Cryptococcus neoformans by BHBM and D2.

FIG. 3. General Synthesis Scheme of acylhydrazones.

FIG. 4. Potential for bioisosterism between ethynyl and halogen substituents.

### DETAILED DESCRIPTION OF THE INVENTION

The present invention provides a compound having the structure:

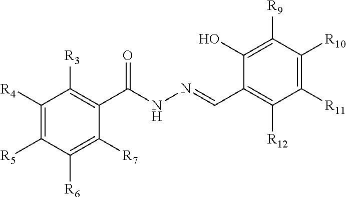

wherein
R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, —CN, alkenyl, alkynyl, aryl, heteroaryl, cycloalkyl, heterocycloalkyl, —OAc, —OR₁₃, —COR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
   wherein when one of R₉, R₁₀, R₁₁, and R₁₂ is —OR₁₃, R₃, R₄, R₅, R₆, and R₇ are not halogen or alkyl.

The present invention provides a compound having the structure:

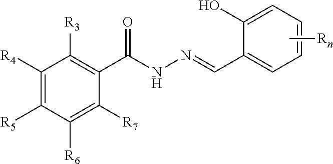

wherein R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
wherein Rₙ is —CN, alkenyl, alkynyl, aryl, heteroaryl, cycloalkyl, heterocycloalkyl, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
wherein when Rₙ is —OR₁₃, R₃, R₄, R₅, R₆, and R₇ are not halogen or alkyl.

In some embodiments the present invention provides a compound having the structure:

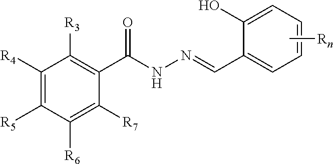

wherein R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
wherein Rₙ is alkenyl, alkynyl, aryl, heteroaryl, cycloalkyl, or heterocycloalkyl.

In some embodiment, when Rₙ is —OR₁₃, R₃, R₄, R₅, R₆, and R₇ are not halogen or alkyl.

In some embodiments, at least two of R₉, R₁₀, R₁₁, and R₁₂ is not H.

In some embodiments, at least two of R₃, R₄, R₅, R₆, and R₇ is not H.

In some embodiments, when R₁₄ is methyl, R₁₅ is not methyl.

In some embodiments, when R₁₅ is methyl, R₁₄ is not methyl.

In some embodiments, at least one of R₉, R₁₀, R₁₁, and R₁₂ is not H.

In some embodiments, at least one of R₃, R₄, R₅, R₆, and R₇ is not H.

In some embodiments, Rₙ is C₁-C₆ alkynyl or aryl.

In some embodiments, Rₙ is C₁-C₆ alkynyl.

In some embodiments, C₁-C₆ alkynyl is ethynyl, prop-1-yne, but-1-yne, but-2-yne, pent-1-yne, pent-2-yne, hex-1-yne, hex-2-yne, hex-3-yne, buta-1,3-diyne, hexa-1,3-diyne, hexa-1,4-diyne, hexa-1,5-diyne, hexa-2,4-diyne, hexa-1,3,5-triyne, 4-methylpent-2-yne, 4-methylpent-1-yne, or 3-methylpent-1-yne.

In some embodiments, Rₙ is unsubstituted or substituted ethynyl.

In some embodiments, Rₙ is unsubstituted ethynyl.

In some embodiments, Rₙ is substituted ethynyl.

In some embodiments, Rₙ is aryl.

In some embodiments, aryl is phenyl, p-toluenyl (4-methylphenyl), naphthyl, tetrahydro-naphthyl, indanyl, biphenyl, phenanthryl, anthryl or acenaphthyl.

In some embodiments, Rₙ is substituted or unsubstituted phenyl.

In some embodiments, Rₙ is unsubstituted phenyl.

In some embodiments, Rₙ is substituted phenyl.

In some embodiments, R₃, R₄, R₅, R₆, and R₇ are each independently, H, halogen, —CN, alkyl, alkenyl, alkynyl, aryl, heteroaryl, heterocycle, —OH, —OAc, —OR₁₃, —COR₁₃, or —CH₂OR₁₃.

In some embodiments, R₃, R₄, R₅, R₆, and R₇ are each independently, H, halogen, —OCF₃, heterocycle, or —CH₂OR₁₃.

In some embodiments, R₃, R₄, R₅, R₆, and R₇ are each independently H or halogen.

In some embodiments, R₃ is halogen, R₄, R₅, R₆, and R₇ are H.

In some embodiments, R₄ is halogen, R₃, R₅, R₆, and R₇ are H.

In some embodiments, R₅ is halogen, R₃, R₄, R₆, and R₇ are H.

In some embodiments, R₆ is halogen, R₃, R₄, R₅, and R₇ are H.

In some embodiments, R₇ is halogen, R₃, R₄, R₅, and R₆ are H.

In some embodiments, R₃ and R₄ are halogen, R₅, R₆, R₇ are H.

In some embodiments, R₃ and R₅ are halogen, R₄, R₆, R₇ are H.

In some embodiments, R₃ and R₆ are halogen, R₄, R₅, R₇ are H.

In some embodiments, R₃ and R₇ are halogen, R₄, R₅, R₆ are H.

In some embodiments, R₄ and R₅ are halogen, R₃, R₆, R₇ are H.

In some embodiments, R₄ and R₆ are halogen, R₃, R₅, R₇ are H.

In some embodiments, R₄ and R₇ are halogen, R₃, R₅, R₆ are H.

In some embodiments, R₅ and R₆ are halogen, R₃, R₄, R₇ are H.

In some embodiments, R₅ and R₇ are halogen, R₃, R₄, R₆ are H.

In some embodiments, R₆ and R₇ are halogen, R₃, R₄, R₅ are H.

In some embodiments, R₄ is OH, R₃, R₅, R₆ and R₇ are each independently H or halogen.

In some embodiments, R₄ is OH, R₃, R₆ and R₇ are H and R₅ is halogen.

In some embodiments, R₄ is OH, R₃, R₅, and R₆ are H, R₇ is halogen.

In some embodiments, R₄ is OH, R₃, R₅, and R₇ are H, R₆ is halogen.

In some embodiments, R₄ is OH, R₃, R₆, and R₇ are H, R₅ is halogen.

In some embodiments, R₄ is OH, R₅, R₆, and R₇ are H, R₃ is halogen.

In some embodiments, R₃ is OH, R₄, R₅, and R₆ are H, R₇ is halogen.

In some embodiments, R₃ is OH, R₅, R₆, and R₇ are H, R₄ is halogen.

In some embodiments, R₃ is OH, R₄, R₆, and R₇ are H, R₅ is halogen.

In some embodiments, R₃ is OH, R₄, R₅, and R₇ are H, R₆ is halogen.

In some embodiments, R₆ is OH, R₃, R₄, and R₅ are H, R₇ is halogen.

In some embodiments, R₆ is OH, R₃, R₄, and R₇ are H, R₅ is halogen.

In some embodiments, R₆ is OH, R₅, R₆, and R₇ are H, R₃ is halogen.

In some embodiments, R₆ is OH, R₃, R₅, and R₇ are H, R₄ is halogen.

In some embodiments, R₇ is OH, R₃, R₄, and R₅ are H, R₆ is halogen.

In some embodiments, R₇ is OH, R₃, R₄, and R₆ are H, R₅ is halogen.

In some embodiments, R₇ is OH, R₄, R₅, and R₆ are H, R₃ is halogen.

In some embodiments, R₇ is OH, R₃, R₅, and R₆ are H, R₄ is halogen.

In some embodiments, R₃ is OH, R₃, R₄, R₅ and R₇ are each independently H or halogen.

In some embodiments, R₃ is OH, R₄ and R₅ are halogen, R₆ and R₇ are H.

In some embodiments, R₃ is OH, R₄ and R₆ are halogen, R₅ and R₇ are H.

In some embodiments, R₃ is OH, R₄ and R₇ are halogen, R₅ and R₆ are H.

In some embodiments, R₃ is OH, R₅ and R₆ are halogen, R₄ and R₇ are H.

In some embodiments, R₃ is OH, R₅ and R₇ are halogen, R₄ and R₆ are H.

In some embodiments, R₃ is OH, R₆ and R₇ are halogen, R₄ and R₅ are H.

In some embodiments, R₄ is OH, R₃ and R₅ are halogen, R₆ and R₇ are H.

In some embodiments, R₄ is OH, R₃ and R₆ are halogen, R₅ and R₇ are H.

In some embodiments, R₄ is OH, R₃ and R₇ are halogen, R₄ and R₆ are H.

In some embodiments, R₄ is OH, R₅ and R₆ are halogen, R₃ and R₇ are H.

In some embodiments, R₄ is OH, R₅ and R₇ are halogen, R₃ and R₆ are H.

In some embodiments, R₄ is OH, R₆ and R₇ are halogen, R₃ and R₅ are H.

In some embodiments, R₅ is OH, R₄ and R₃ are halogen, R₆ and R₇ are H.

In some embodiments, R₅ is OH, R₄ and R₆ are halogen, R₃ and R₇ are H.

In some embodiments, R₅ is OH, R₄ and R₇ are halogen, R₅ and R₃ are H.

In some embodiments, R₅ is OH, R₃ and R₆ are halogen, R₄ and R₇ are H.

In some embodiments, R₅ is OH, R₃ and R₇ are halogen, R₄ and R₆ are H.

In some embodiments, R₅ is OH, R₆ and R₇ are halogen, R₃ and R₄ are H.

In some embodiments, R₆ is OH, R₄ and R₃ are halogen, R₅ and R₇ are H.

In some embodiments, R₆ is OH, R₄ and R₅ are halogen, R₃ and R₇ are H.

In some embodiments, R₆ is OH, R₄ and R₇ are halogen, R₅ and R₃ are H.

In some embodiments, R₆ is OH, R₃ and R₅ are halogen, R₄ and R₇ are H.

In some embodiments, R₆ is OH, R₃ and R₇ are halogen, R₄ and R₅ are H.

In some embodiments, R₆ is OH, R₅ and R₇ are halogen, R₃ and R₄ are H.

In some embodiments, R₇ is OH, R₄ and R₅ are halogen, R₃ and R₆ are H.

In some embodiments, R₇ is OH, R₄ and R₆ are halogen, R₃ and R₅ are H.

In some embodiments, R₇ is OH, R₃ and R₄ are halogen, R₅ and R₆ are H.

In some embodiments, R₇ is OH, R₃ and R₅ are halogen, R₄ and R₆ are H.

In some embodiments, R₇ is OH, R₃ and R₆ are halogen, R₄ and R₅ are H.

In some embodiments, R₇ is OH, R₅ and R₆ are halogen, R₃ and R₄ are H.

In some embodiments, halogen is F, Cl, Br, and I.

In some embodiments, halogen is Br.

In some embodiments, halogen is F.

In some embodiments, halogen is Cl.

In some embodiments, halogen is I.

In some embodiments, R₃, R₄, R₅, R₆, R₇ are each independently, H or —CH₂OR₁₃.

In some embodiments, R₃ is —CH₂OR₁₃, R₄, R₅, R₆, and R₇ are H.

In some embodiments, R₄ is —CH₂OR₁₃, R₃, R₅, R₆, and R₇ are H.

In some embodiments, R₅ is —CH₂OR₁₃, R₃, R₄, R₆, and R₇ are H.

In some embodiments, R₆ is —CH₂OR₁₃, R₃, R₄, R₅, and R₇ are H.

In some embodiments, R₇ is —CH₂OR₁₃, R₃, R₄, R₅, and R₆ are H.

In some embodiments, R₃ and R₄ are —CH₂OR₁₃, R₅, R₆, R₇ are H.

In some embodiments, R₃ and R₅ are —CH₂OR₁₃, R₄, R₆, R₇ are H.

In some embodiments, R₃ and R₆ are —CH₂OR₁₃, R₄, R₅, R₇ are H.

In some embodiments, R₃ and R₇ are —CH₂OR₁₃, R₄, R₅, R₆ are H.

In some embodiments, R₄ and R₅ are —CH₂OR₁₃, R₃, R₆, R₇ are H.

In some embodiments, R₄ and R₆ are —CH₂OR₁₃, R₃, R₅, R₇ are H.

In some embodiments, R₄ and R₇ are —CH₂OR₁₃, R₃, R₅, R₆ are H.

In some embodiments, R₅ and R₆ are —CH₂OR₁₃, R₃, R₄, R₇ are H.

In some embodiments, R₅ and R₇ are —CH₂OR₁₃, R₃, R₄, R₆ are H.

In some embodiments, R₆ and R₇ are —CH₂OR₁₃, R₃, R₄, R₅ are H.

In some embodiments, R₁₃ is alkyl.

In some embodiments, alkyl is C₁-C₆ alkyl, branched or unbranched.

In some embodiments, alkyl is ethyl, or branched or unbranched propyl.

In some embodiments, alkyl is methyl, ethyl, propyl, or isopropyl.

In some embodiments, R₃, R₄, R₅, R₆, and R₇ are each independently, H, halogen or —OCF₃.

In some embodiments, R₃ is halogen, R₄, R₅, and R₆ are H, R₇ is —OCF₃.

In some embodiments, R₃ is halogen, R₅, R₆, and R₇ are H, R₄ is —OCF₃.

In some embodiments, R₃ is halogen, R₄, R₆, and R₇ are H, R₅ is —OCF₃.

In some embodiments, R₃ is halogen, R₄, R₅, and R₇ are H, R₆ is —OCF₃.

In some embodiments, R₄ is halogen, R₃, R₅, and R₆ are H, R₇ is —OCF₃.

In some embodiments, R₄ is halogen, R₃, R₅, and R₇ are H, R₆ is —OCF₃.

In some embodiments, R₄ is halogen, R₃, R₆, and R₇ are H, R₅ is —OCF₃.

In some embodiments, R₄ is halogen, R₅, R₆, and R₇ are H, R₃ is —OCF₃.

In some embodiments, R₆ is halogen, R₃, R₄, and R₅ are H, R₇ is —OCF₃.

In some embodiments, R₆ is halogen, R₃, R₄, and R₇ are H, R₅ is —OCF₃.

In some embodiments, R₆ is halogen, R₅, R₆, and R₇ are H, R₃ is —OCF₃.

In some embodiments, R₆ is halogen, R₃, R₅, and R₇, are H, R₄ is —OCF₃.

In some embodiments, R₇ is halogen, R₃, R₄, and R₅ are H, R₆ is —OCF₃.

In some embodiments, R₇ is halogen, R₃, R₄, and R₆ are H, R₅ is —OCF₃.

In some embodiments, R₇ is halogen, R₄, R₅, and R₆ are H, R₃ is —OCF₃.

In some embodiments, R₇ is halogen, R₃, R₅, and R₆ are H, R₄ is —OCF₃.

In some embodiments, halogen is F, Cl, Br, or I.

In some embodiments, halogen is F.

In some embodiments, R₃, R₄, R₅, R₆, and R₇ are each H or heterocycle.

In some embodiments, R₃ is heterocycle, R₄, R₅, R₆, and R₇ are H.

In some embodiments, R₄ is heterocycle, R₃, R₅, R₆, and R₇ are H.

In some embodiments, R₅ is heterocycle, R₃, R₄, R₆, and R₇ are H.

In some embodiments, R₆ is heterocycle, R₃, R₄, R₅, and R₇ are H.

In some embodiments, R₇ is heterocycle, R₃, R₄, R₅, and R₆ are H.

In some embodiments, R₃ and R₄ are heterocycle, R₅, R₆, R₇ are H.

In some embodiments, R₃ and R₅ are heterocycle, R₄, R₆, R₇ are H.

In some embodiments, R₃ and R₆ are heterocycle, R₄, R₅, R₇ are H.

In some embodiments, R₃ and R₇ are heterocycle, R₄, R₅, R₆ are H.

In some embodiments, R₄ and R₅ are heterocycle, R₃, R₆, R₇ are H.

In some embodiments, R₄ and R₆ are heterocycle, R₃, R₅, R₇ are H.

In some embodiments, R₄ and R₇ are heterocycle, R₃, R₅, R₆ are H.

In some embodiments, R₅ and R₆ are heterocycle, R₃, R₄, R₇ are H.

In some embodiments, R₅ and R₇ are heterocycle, R₃, R₄, R₆ are H.

In some embodiments, R₆ and R₇ are heterocycle, R₃, R₄, R₅ are H.

In some embodiments, R₆ and R₇ are heterocycle, R₃, R₄, R₅ are H.

In some embodiments, heterocycle is three to four-membered and has one or more degrees of unsaturation.

In some embodiments, heterocycle is three to four-membered and has one degrees of unsaturation.

In some embodiments, heterocycle is three-membered and has one or more degrees of unsaturation.

In some embodiments, heterocycle is three-membered and has one degree of unsaturation.

In some embodiments, heterocycle is aziridine, 2H-azirine, oxirane, thiirane or azirine.

In some embodiments, azirine is 3-methyl-3-(trifluoromethyl)-3H-diazirine.

The present invention provides a compound having the structure

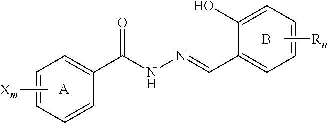

wherein ring A has the following structure:

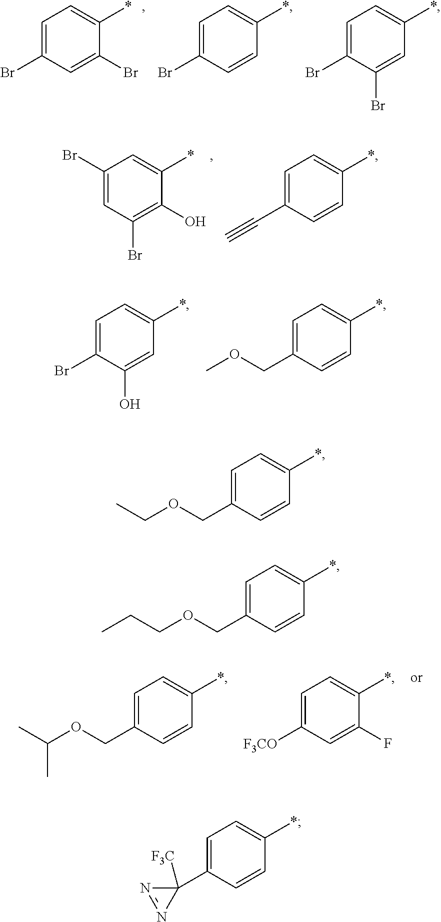

wherein Ring B has the following structure:

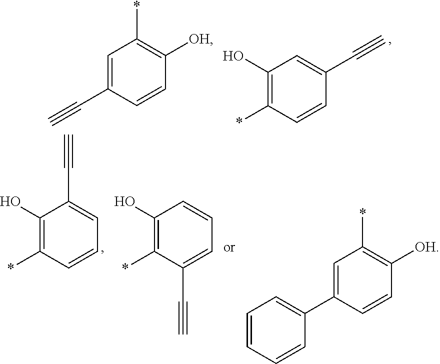

The present invention provides a compound having the structure:

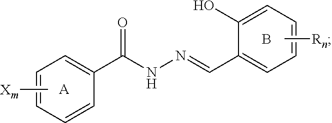

wherein Ring A has the following structure:

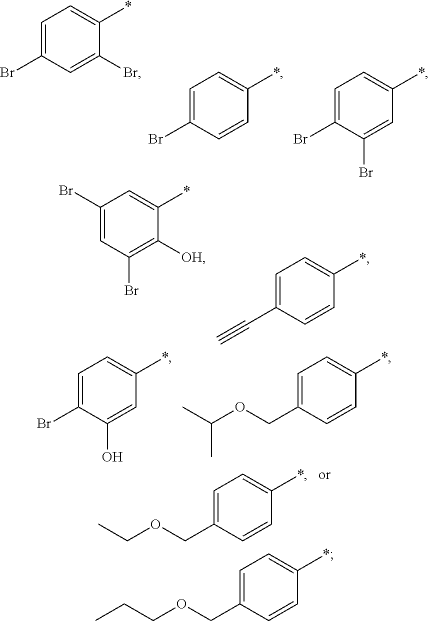

wherein Ring B has the following structure:

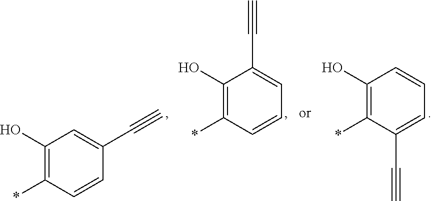

The present invention provides a compound having the structure:

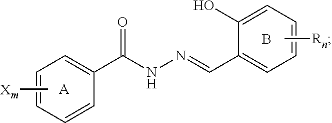

wherein Ring A has the following structure:

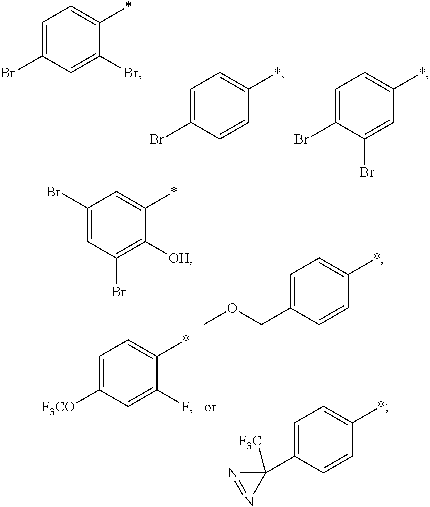

wherein Ring B has the following structure:

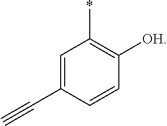 [SMILES: *c1cc(C#C)ccc1O]

The present invention provides a compound having the structure:

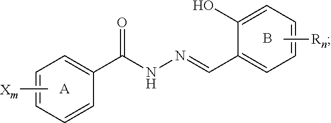

wherein Ring A has the following structure:

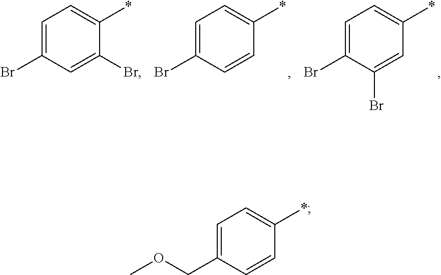

wherein Ring B has the following structure:

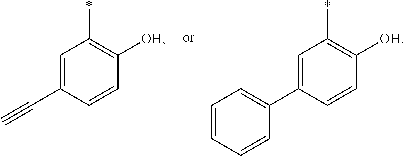

The present invention provides a compound having the structure:

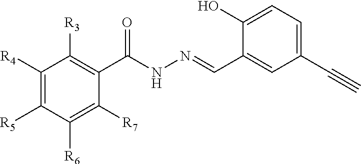

wherein R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₃, R₄, R₅, R₆, and R₇ is not H.

The present invention provides a compound having the structure:

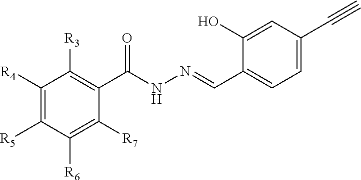

wherein R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₃, R₄, R₅, R₆, and R₇ is not H.

The present invention provides a compound having the structure:

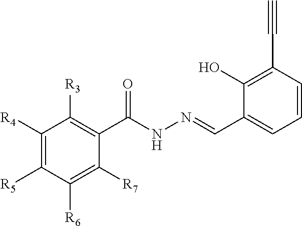

wherein R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₃, R₄, R₅, R₆, and R₇ is not H.

The present invention provides a compound having the structure:

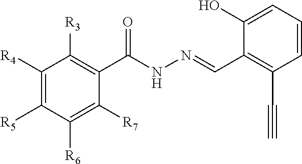

wherein R₃, R₄, R₅, R₆ and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₃, R₄, R₅, R₆, and R₇ is not H.

The present invention provides a compound having the structure:

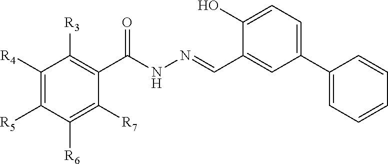

wherein R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₃, R₄, R₅, R₆, and R₇ is not H.

The present invention provides a compound having the structure:

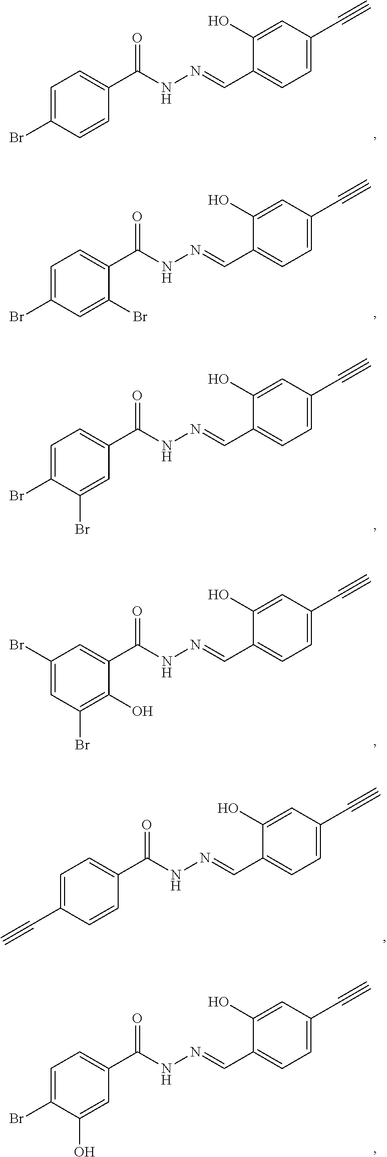

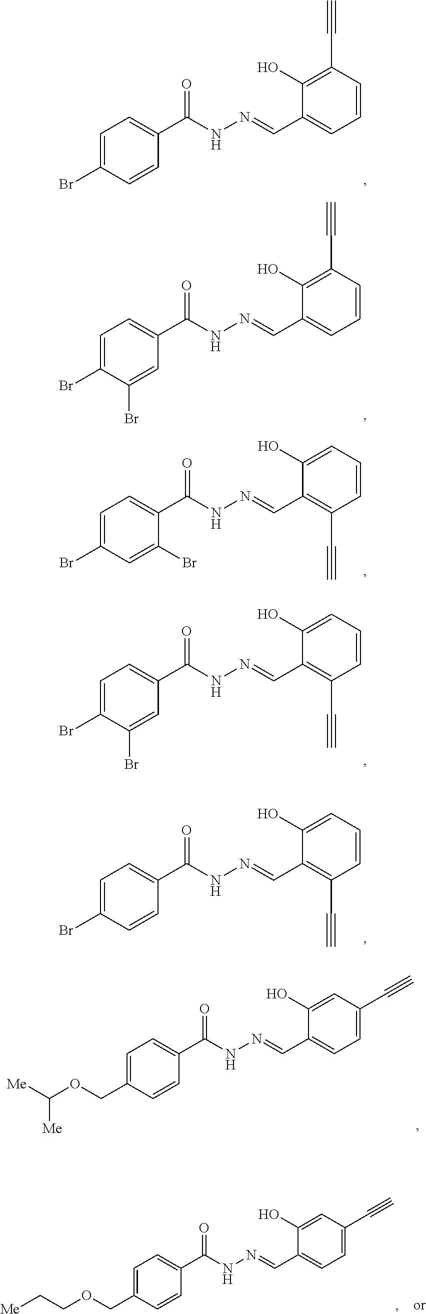

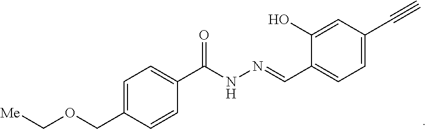 [SMILES: C#Cc1ccc(/C=N/NC(=O)c2ccc(COCC)cc2)c(O)c1]

The present invention provides a compound having the structure:

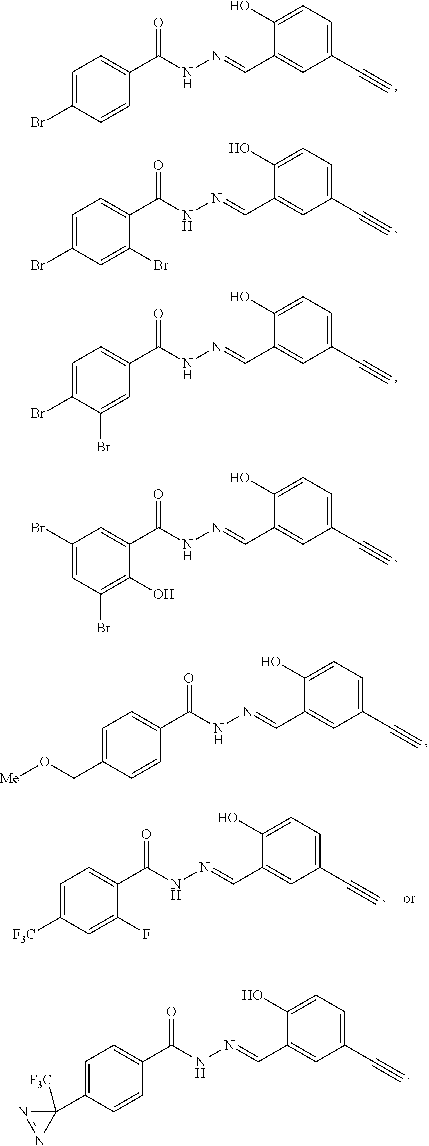

The present invention provides a compound having the structure:

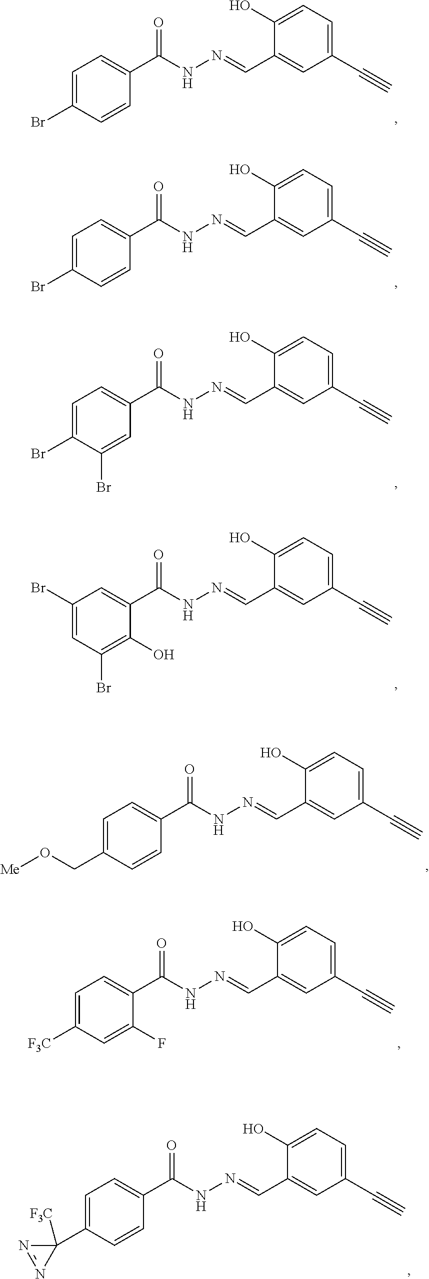

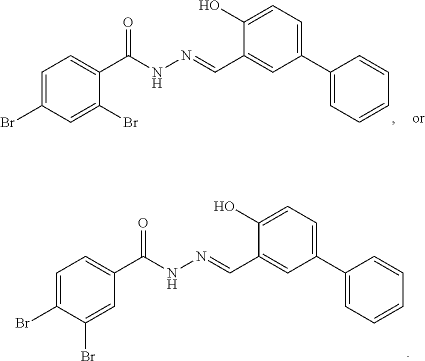 [SMILES: O=C(N/N=C/c1cc(-c2ccccc2)ccc1O)c1ccc(Br)cc1Br ; O=C(N/N=C/c1cc(-c2ccccc2)ccc1O)c1ccc(Br)c(Br)c1]

The present invention provides a compound having the structure:

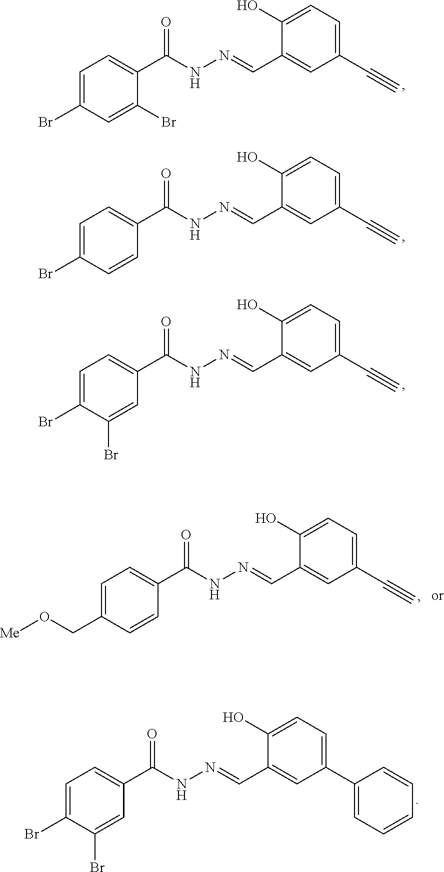 [SMILES: C#Cc1ccc(O)c(/C=N/NC(=O)c2ccc(Br)cc2Br)c1 ; C#Cc1ccc(O)c(/C=N/NC(=O)c2ccc(Br)cc2)c1 ; C#Cc1ccc(O)c(/C=N/NC(=O)c2ccc(Br)c(Br)c2)c1 ; C#Cc1ccc(O)c(/C=N/NC(=O)c2ccc(COC)cc2)c1 ; O=C(N/N=C/c1cc(-c2ccccc2)ccc1O)c1ccc(Br)c(Br)c1]

The present invention provides a compound having the structure:

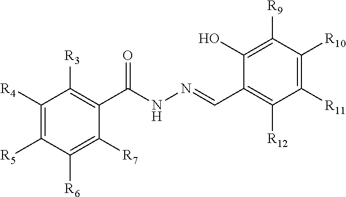

wherein
R₃, R₄, R₅, R₆ and R₇ are each independently H, alkenyl, alkynyl, aryl, heteroaryl, —OAc, —COR₁₃, —CH₂OR₁₃, —SH, —SR₁₃, —SO₂R₁₃, —NH₂, —NR₁₄R₁₅, —NHCOR₁₂, or —CONR₁₄R₁₅;
R₉, R₁₀, R₁₁, R₁₂ are each independently —H, halogen, —CN, —CF₃, —OCF₃, —NO₂, alkyl, alkenyl, alkynyl, aryl, heteroaryl, —OH, —OAc, —OR₁₃, —COR₁₃, —SH, —SR₁₃, —SO₂R₁₃, —SO₂NR₁₄R₁₅, —NH₂, —NHR₁₃, NR₁₄R₁₅, —NHCOR₁₂, or —CONR₁₄R₁₅;
wherein each occurrence of R₁₃ is independently alkyl, alkenyl, alkynyl, aryl, or heteroaryl;
wherein each occurrence of R₁₄ is independently —H, alkyl, alkenyl, alkynyl, aryl, or heteroaryl;
wherein each occurrence of R₁₅ is independently —H, alkyl, alkenyl, alkynyl, aryl, or heteroaryl;
wherein when R₁₄ is H, R₁₅ is not methyl or when R₁₅ is H, R₁₄ is not methyl;
wherein at least one of R₉, R₁₀, R₁₁, and R₁₂ is not H;
wherein at least one of R₃, R₄, R₅, R₆, and R₇ is not H.

In some embodiments, at least two of R₉, R₁₀, R₁₁, and R₁₂ is not H.

In some embodiments, at least two of R₃, R₄, R₅, R₆, and R₇ is not H.

In some embodiments, R₃, R₄, R₅, R₆, and R₇ are each independently H, alkenyl, alkynyl, aryl, heteroaryl, —OAc, or —CH₂OR₁₃.

In some embodiments, R₃, R₄, R₅, R₆, and R₇ are each independently H, alkynyl, or —CH₂OR₁₃.

In some embodiments, R₃, R₄, R₅, R₆, and R₇ are independently H or —CH₂OR₁₃.

In some embodiments, R₃ is —CH₂OR₁₃, R₄, R₅, R₆, and R₇ are H.

In some embodiments, R₄ is —CH₂OR₁₃, R₃, R₅, R₆, and R₇ are H.

In some embodiments, R₅ is —CH₂OR₁₃, R₃, R₄, R₆, and R₇ are H.

In some embodiments, R₆ is —CH₂OR₁₃, R₃, R₄, R₅, and R₇ are H.

In some embodiments, R₇ is —CH₂OR₁₃, R₃, R₄, R₅, and R₆ are H.

In some embodiments, R₃ and R₄ are —CH₂OR₁₃, R₅, R₆, R₇ are H.

In some embodiments, R₃ and R₅ are —CH₂OR₁₃, R₄, R₆, R₇ are H.

In some embodiments, R₃ and R₆ are —CH₂OR₁₃, R₄, R₅, R₇ are H.

In some embodiments, R₃ and R₇ are —CH₂OR₁₃, R₄, R₅, R₆ are H.

In some embodiments, R₄ and R₅ are —CH₂OR₁₃, R₃, R₆, R₇ are H.

In some embodiments, R₄ and R₆ are —CH₂OR₁₃, R₃, R₅, R₇ are H.

In some embodiments, R₄ and R₇ are —CH₂OR₁₃, R₃, R₅, R₆ are H.

In some embodiments, R₅ and R₆ are —CH₂OR₁₃, R₃, R₄, R₇ are H.

In some embodiments, R₅ and R₇ are —CH₂OR₁₃, R₃, R₄, R₆ are H.

In some embodiments, R₆ and R₇ are —CH₂OR₁₃, R₃, R₄, R₅ are H.

In some embodiments, R₁₃ is alkyl.

In some embodiments, R₃ is C₂-C₆ branched or unbranched.

In some embodiments, R₁₃ is methyl, ethyl, propyl, or isopropyl.

In some embodiments, R₁₃ is ethyl or propyl.

In some embodiments, R₁₃ is methyl.

In some embodiments, R₁₃ is ethyl.

In some embodiments, R₁₃ is propyl.

In some embodiments, R₁₃ is isopropyl.

In some embodiments, R₉, R₁₀, R₁₁, R₁₂ are each independently H, halogen, —CN, —CF₃, —OCF₃, —NO₂, alkyl, alkenyl, alkynyl, aryl, heteroaryl, —OH.

In some embodiments, R₉, R₁₀, R₁₁, R₁₂ are each independently halogen or H.

In some embodiments, R₉ is halogen, R₁₀, R₁₁, and R₁₂ are H.

In some embodiments, R₁₀ is halogen, R₉, R₁₁, and R₁₂ are H.

In some embodiments, R₁₁ is halogen, R₉, R₁₀, and R₁₂ are H.

In some embodiments, R₁₂ is halogen, R₉, R₁₀, and R₁₁ are H.

In some embodiments, R₉ and R₁₀ are halogen, R₁₁ and R₁₂ are H.

In some embodiments, R₉ and R₁₁ are halogen, R₁₀ and R₁₂ are H.

In some embodiments, R₉ and R₁₂ are halogen, R₁₀ and R₁₁ are H.

In some embodiments, R₁₀ and R₁₁ are halogen, R₉ and R₁₀ are H.

In some embodiments, R₁₀ and R₁₂ are halogen, R₉ and R₁₁ are H.

In some embodiments, R₁₁ and R₁₂ are halogen, R₉ and R₁₀ are H.

In some embodiments, halogen is F, Br, Cl, or I.

In some embodiments, halogen is Br.

The present invention provides a compound having the structure

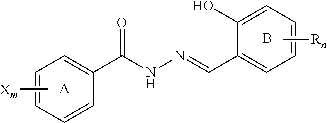

wherein Ring A has the following structure:

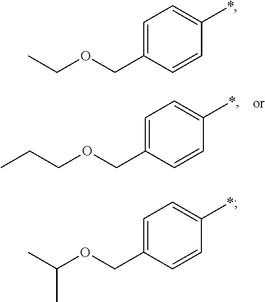

wherein Ring B has the following structure:

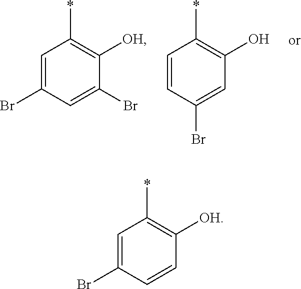

The present invention provides a compound having the structure:

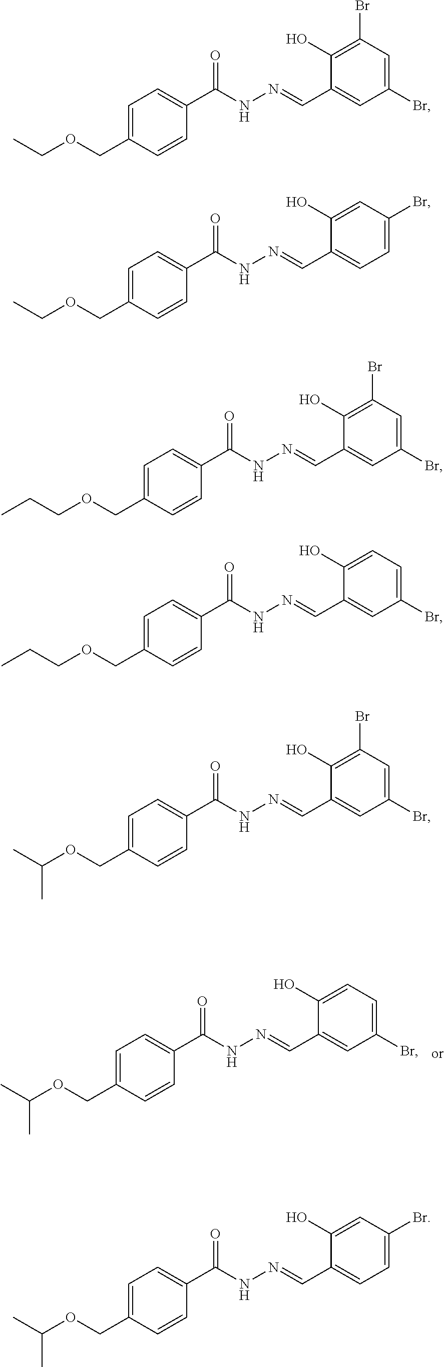

The present invention provides a pharmaceutical composition comprising the compound disclosed in the present invention and a pharmaceutically acceptable carrier.

The present invention provides a method of inhibiting the growth of a fungus comprising contacting the fungus with an effective amount of a compound having the structure:

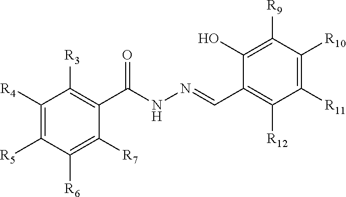

wherein
R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, —CN, alkenyl, alkynyl, aryl, heteroaryl, cycloalkyl, heterocycloalkyl, —OAc, —COR₁₃, —OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
   wherein when one of R₉, R₁₀, R₁₁, and R₁₂ is —OR₁₃, R₃, R₄, R₅, R₆, and R₇ are not halogen or alkyl;
or a pharmaceutically acceptable salt or ester thereof, so as to thereby inhibit the growth of the fungus.

The present invention provides a method of inhibiting fungal sphingolipid synthesis in a fungus comprising contacting the fungus with an effective amount of a compound having the structure:

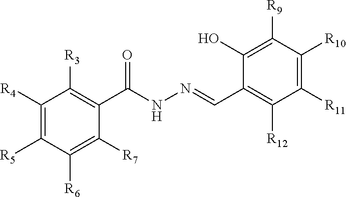

wherein
R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, —CN, alkenyl, alkynyl, aryl, heteroaryl, cycloalkyl, heterocycloalkyl, —OAc, —COR₁₃, —OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
   wherein when one of R₉, R₁₀, R₁₁, and R₁₂ is —OR₁₃, R₃, R₄, R₅, R₆, and R₇ are not halogen or alkyl;
or a pharmaceutically acceptable salt or ester thereof, so as to thereby inhibit sphingolipid synthesis in the fungus.

The present invention provides a method of inhibiting fungal sphingolipid synthesis in a fungus in a mammal without substantially inhibiting mammalian sphingolipid synthesis comprising administering to the mammal an effective amount of a compound having the structure:

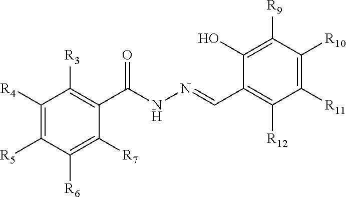

wherein
R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, —CN, alkenyl, alkynyl, aryl, heteroaryl, cycloalkyl, heterocycloalkyl, —OAc, —COR₁₃, —OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
wherein when one of R₉, R₁₀, R₁₁, and R₁₂ is —OR₁₃, R₃, R₄, R₅, R₆, and R₇ are not halogen or alkyl;
or a pharmaceutically acceptable salt or ester thereof, so as to thereby inhibit fungal sphingolipid synthesis in the fungus in the mammal without substantially inhibiting mammalian sphingolipid synthesis.

The present invention provides a method of inhibiting the growth of a fungus comprising contacting the fungus with an effective amount of a compound having the structure:

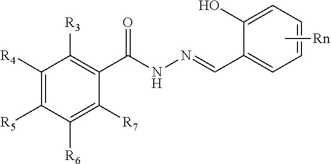

wherein R₃, R₄, R₅, R₆ and R₇ are each independently H halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
wherein Rₙ is alkenyl, alkynyl, aryl, heteroaryl, cycloalkyl, or heterocycloalkyl;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₃, R₄, R₅, R₆, and R₇ is not H.

The present invention provides a method of inhibiting the growth of a fungus comprising contacting the fungus with an effective amount of a compound having the structure:

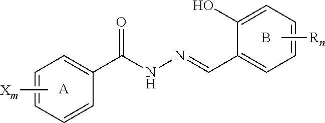

wherein ring A has the following structure:

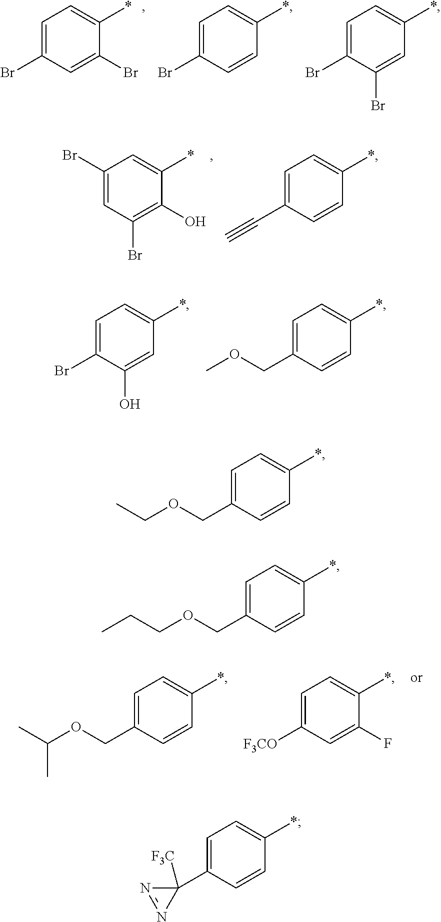

wherein ring B has the following structure:

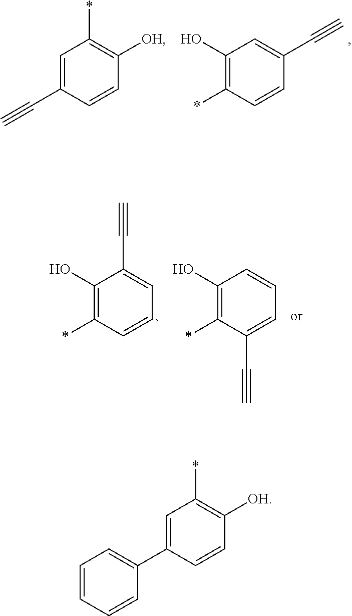

The present invention provides a method of inhibiting the growth of a fungus comprising contacting the fungus with an effective amount of a compound having the structure:

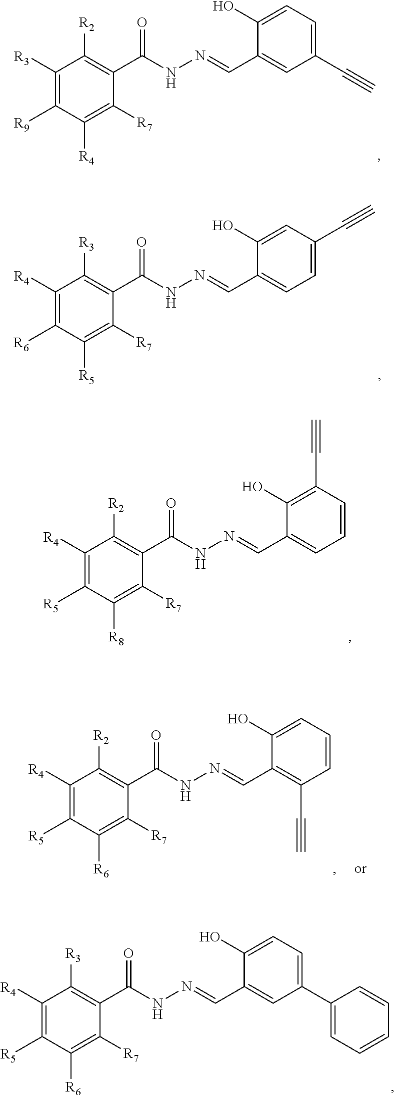

wherein R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is methyl, R₁₅ is not methyl;
wherein at least one of R₃, R₄, R₅, R₆, and R₇ is not H.

The present invention provides a method of inhibiting the growth of a fungus comprising contacting the fungus with an effective amount of a compound having the structure:

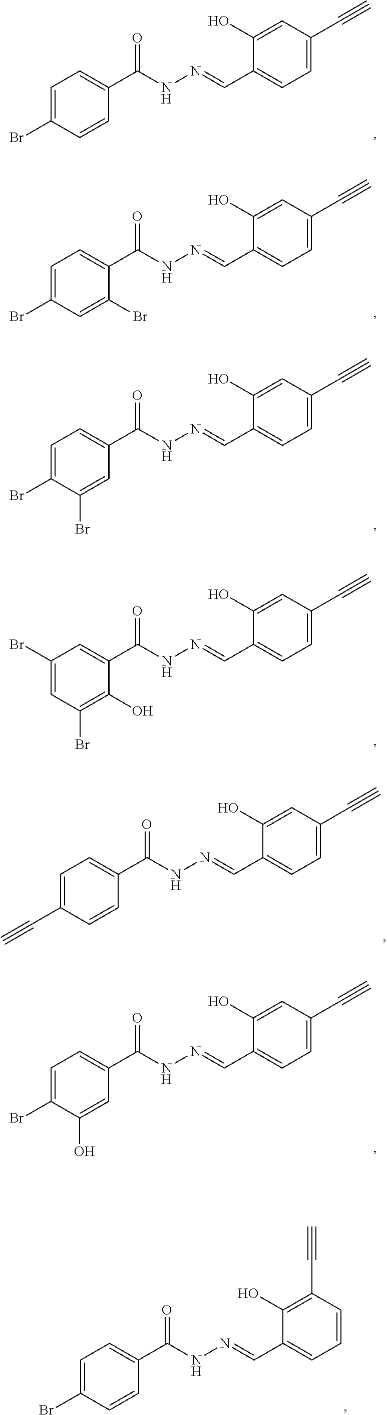

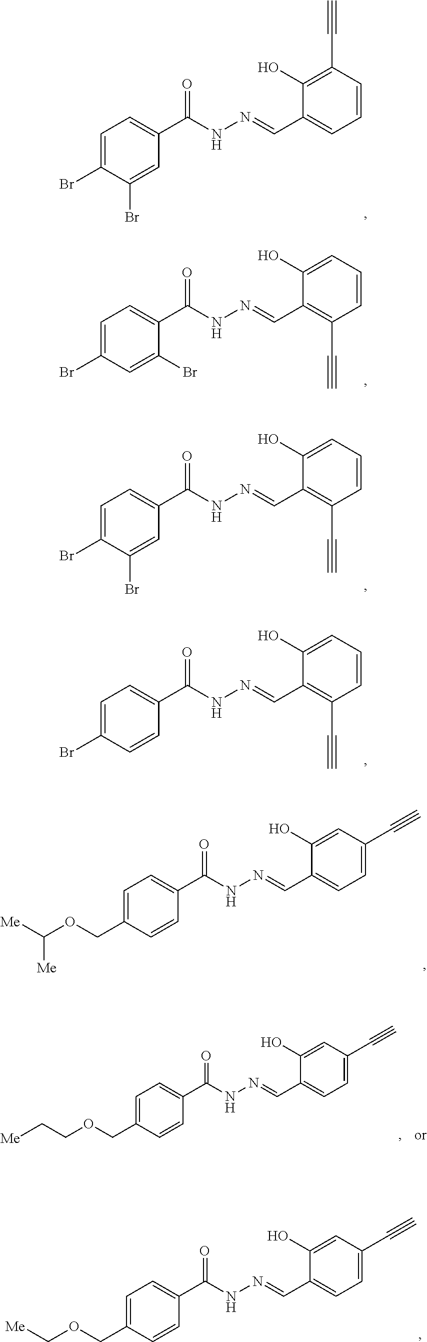

The present invention provides a method of inhibiting the growth of a fungus comprising contacting the fungus with an effective amount of a compound having the structure having the structure:

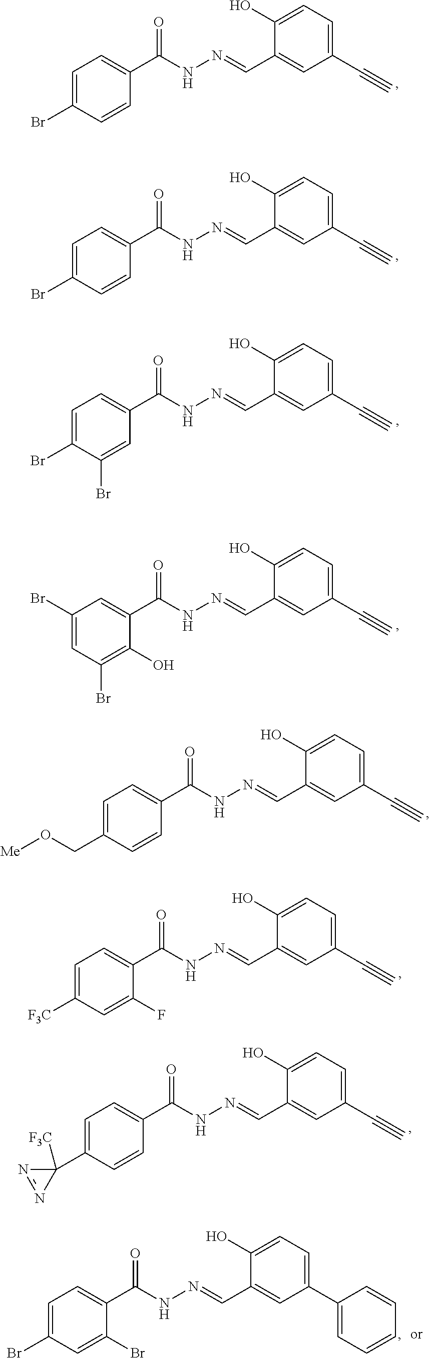

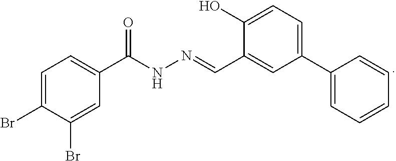 [SMILES: O=C(N/N=C/c1cc(-c2ccccc2)ccc1O)c1ccc(Br)c(Br)c1]

The present invention provides a method of inhibiting the growth of a fungus comprising contacting the fungus with an effective amount of a compound having the structure having the structure:

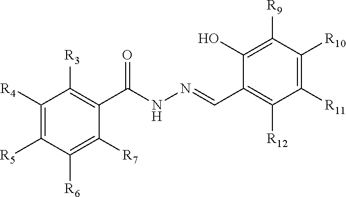

wherein
   R₃, R₄, R₅, R₆, and R₇ are each independently H, alkenyl, alkynyl, aryl, heteroaryl, heterocycle, —OAc, —COR₁₃, —CH₂OR₁₃, —SH, —SR₁₃, —SO₂R₁₃, —NH₂, —NR₁₄R₁₅, —NHCOR₁₂, or —CONR₁₄R₁₅;
   R₉, R₁₀, R₁₁, R₁₂ are each independently —H, halogen, —CN, —CF₃, —OCF₃, —NO₂, alkyl, alkenyl, alkynyl, aryl, heteroaryl, —OH, —OAc, —OR₁₃, —COR₁₃, —SH, —SR₁₃, —SO₂R₁₃, —SO₂NR₁₄R₁₅, —NH₂, —NHR₁₃, NR₁₄R₁₅, —NHCOR₁₂, or —CONR₁₄R₁₅;
   wherein each occurrence of R₁₃ is independently alkyl, alkenyl, alkynyl, aryl, or heteroaryl;
   wherein each occurrence of R₁₄ is independently —H, alkyl, alkenyl, alkynyl, aryl, or heteroaryl;
   wherein each occurrence of R₁₅ is independently —H, alkyl, alkenyl, alkynyl, aryl, or heteroaryl;
wherein when R₁₄ is H, R₁₅ is not methyl or when R₁₅ is H, R₁₄ is not methyl;
wherein at least one of R₉, R₁₀, R₁₁, and R₁₂ is not H;
wherein at least one of R₃, R₄, R₅, R₆, and R₇ is not H;
or a pharmaceutically acceptable salt or ester thereof, so as to thereby inhibit the growth of the fungus.

The present invention provides a method of inhibiting fungal sphingolipid synthesis in a fungus comprising contacting the fungus with an effective amount of a compound having the structure:

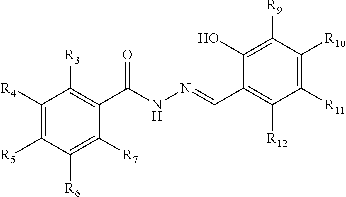

wherein
R₃, R₄, R₅, R₆ and R₇ are each independently H, alkenyl, alkynyl, aryl, heteroaryl, heterocycle, —OAc, —COR₁₃, —CH₂OR₁₃, —SH, —SR₁₃, —SO₂R₁₃, —NH₂, —NR₁₄R₁₅, —NHCOR₁₂, or —CONR₁₄R₁₅;
R₉, R₁₀, R₁₁, R₁₂ are each independently —H, halogen, —CN, —CF₃, —OCF₃, —NO₂, alkyl, alkenyl, alkynyl, aryl, heteroaryl, —OH, —OAc, —OR₁₃, —COR₁₃, —SH, —SR₁₃, —SO₂R₁₃, —SO₂NR₁₄R₁₅, —NH₂, —NHR₁₃, NR₁₄R₁₅, —NHCOR₁₂, or —CONR₁₄R₁₅;
wherein each occurrence of R₁₃ is independently alkyl, alkenyl, alkynyl, aryl, or heteroaryl;
wherein each occurrence of R₁₄ is independently —H, alkyl, alkenyl, alkynyl, aryl, or heteroaryl;
wherein each occurrence of R₁₅ is independently —H, alkyl, alkenyl, alkynyl, aryl, or heteroaryl;
wherein when R₁₄ is H, R₁₅ is not methyl or when R₁₅ is H, R₁₄ is not methyl;
wherein at least one of R₉, R₁₀, R₁₁, and R₁₂ is not H;
wherein at least one of R₃, R₄, R₅, R₆, and R₇ is not H;
or a pharmaceutically acceptable salt or ester thereof, so as to thereby inhibit sphingolipid synthesis in the fungus.

The present invention provides a method of inhibiting fungal sphingolipid synthesis in a fungus in a mammal without substantially inhibiting mammalian sphingolipid synthesis comprising administering to the mammal an effective amount of a compound having the structure:

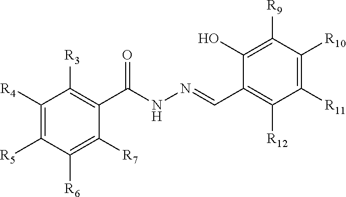

wherein
R₃, R₄, R₅, R₆, and R₇ are each independently H, alkenyl, alkynyl, aryl, heteroaryl, heterocycle, —OAc, —COR₁₃, —CH₂OR₁₃, —SH, —SR₁₃, —SO₂R₁₃, —CH₂OR₁₃, —NH₂, —NR₁₄R₁₅, —NHCOR₁₂, or —CONR₁₄R₁₅;
R₉, R₁₀, R₁₁, R₁₂ are each independently —H, halogen, —CN, —CF₃, —OCF₃, —NO₂, alkyl, alkenyl, alkynyl, aryl, heteroaryl, —OH, —OAc, —OR₁₃, —COR₁₃, —SH, —SR₁₃, —SO₂R₁₃, —SO₂NR₁₄R₁₅, —NH₂, —NHR₁₃, NR₁₄R₁₅, —NHCOR₁₂, or —CONR₁₄R₁₅;
wherein each occurrence of R₁₃ is independently alkyl, alkenyl, alkynyl, aryl, or heteroaryl;
wherein each occurrence of R₁₄ is independently —H, alkyl, alkenyl, alkynyl, aryl, or heteroaryl;
wherein each occurrence of R₁₅ is independently —H, alkyl, alkenyl, alkynyl, aryl, or heteroaryl;
wherein when R₁₄ is H, R₁₅ is not methyl or when R₁₅ is H, R₁₄ is not methyl;
wherein at least one of R₉, R₁₀, R₁₁, and R₁₂ is not H;
wherein at least one of R₃, R₄, R₅, R₆, and R₇ is not H;
or a pharmaceutically acceptable salt or ester thereof, so as to thereby inhibit fungal sphingolipid synthesis in the fungus in the mammal without substantially inhibiting mammalian sphingolipid synthesis.

The present invention provides a method of inhibiting the growth of a fungus comprising contacting the fungus with an effective amount of a compound having the structure having the structure:

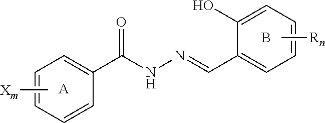

wherein Ring A has the following structure:

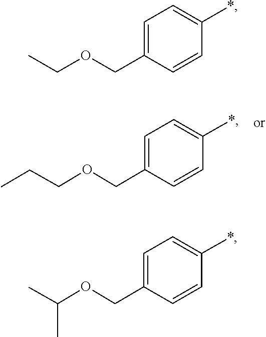

wherein Ring B has the following structure:

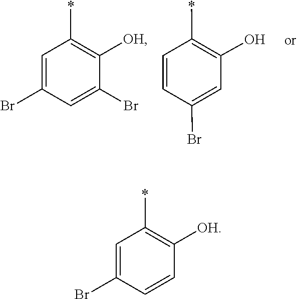

The present invention provides a method of inhibiting the growth of a fungus comprising contacting the fungus with an effective amount of a compound having the structure:

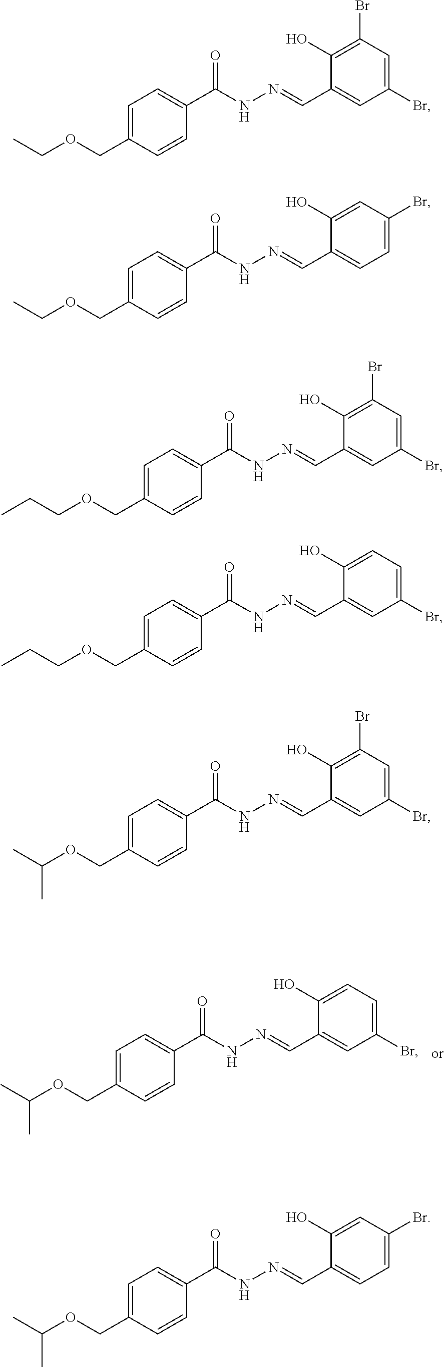

In some embodiments of the method, Rₙ is C₁-C₆ alkynyl or aryl.

In some embodiments of the method, Rₙ is C₁-C₆ alkynyl.

In some embodiments of the method, C₁-C₆ alkynyl is ethynyl, prop-1-yne, but-1-yne, but-2-yne, pent-1-yne, pent-2-yne, hex-1-yne, hex-2-yne, hex-3-yne, buta-1,3-diyne, hexa-1,3-diyne, hexa-1,4-diyne, hexa-1,5-diyne, hexa-2,4-diyne, hexa-1,3,5-triyne, 4-methylpent-2-yne, 4-methylpent-1-yne, or 3-methylpent-1-yne.

In some embodiments of the method, Rₙ is unsubstituted or substituted ethynyl.

In some embodiments of the method, Rₙ is unsubstituted ethynyl.

In some embodiments of the method, Rₙ is substituted ethynyl.

In some embodiments of the method, Rₙ is aryl.

In some embodiments of the method, aryl is phenyl, ρ-toluenyl (4-methylphenyl), naphthyl, tetrahydro-naphthyl, indanyl, biphenyl, phenanthryl, anthryl or acenaphthyl.

In some embodiments of the method, Rₙ is substituted or unsubstituted phenyl.

In some embodiments of the method, Rₙ is unsubstituted phenyl.

In some embodiments of the method, Rₙ is substituted phenyl.

In some embodiments of the method, heterocycle is three to four-membered and has one or more degrees of unsaturation.

In some embodiments of the method, heterocycle is three to four-membered and has one degrees of unsaturation.

In some embodiments of the method, heterocycle is three-membered and has one or more degrees of unsaturation.

In some embodiments of the method, heterocycle is three-membered and has one degree of unsaturation.

In some embodiments of the method, heterocycle is aziridine, azirine, diazirine, oxirane, thiirane, azetidine, oxetane, thietane.

In some embodiments of the method, heterocycle is aziridine.

In some embodiments of the method, heterocycle is azirine.

In some embodiments of the method, the method further comprises inhibiting the growth of a fungus in a plant.

In some embodiments of the method, the method further comprising contacting the fungus with an effective amount of an anti-fungal agent.

In some embodiments of the method, the method further comprising administering to the mammal an effective amount of an anti-fungal agent.

In some embodiments of the method, the method further comprising administering to a plant an effective amount of an anti-fungal agent.

In some embodiments of the method, the amount of the compound and the amount of the anti-fungal agent when taken together is more effective to inhibit the growth of the fungus than the anti-fungal agent alone, or more effective to inhibit fungal sphingolipid synthesis than the anti-fungal agent alone.

In some embodiments of the method, the amount of the compound and the amount of the anti-fungal agent when taken together is more effective to inhibit fungal sphingolipid synthesis without substantially inhibiting mammalian sphingolipid synthesis in the mammal than the anti-fungal agent alone.

In some embodiments of the method, the anti-fungal agent is fluconazole, amphotericin B, caspofungin, tunicamycin or aureobasidin A.

In some embodiments of the method, the fungus is Cryptococcus Neoformans, Cryptococcus Neoformans, Cryptococcus gattii, Candida albicans, Candida krusei, Candida glabrata, Candida parapsilosis, Candida guilliermondii, Aspergillus fumigatus, Rhizopus oryzae, Rhizopus spp., Blastomyces dermatitis, Histoplasma capsulatum, Coccidioides spp., Paecilomyces variotii, Pneumocystis murina, Pneumocystis jiroveci, Histoplasma capsulatum, Aspergillus spp., Sporothrix brasiliensis, S. schenckii, S. globosa, S. mexicana, S. chilensis, S. luriei, or S. pallida.

In some embodiments of the method, the fungus is Cryptococcus Neoformans.

In some embodiments of the method, the fungus is Sporothrix brasiliensis.

In some embodiments of the method, the fungus is other than Cryptococcus Neoformans.

In some embodiments of the method, the fungus is Cryptococcus gattii, Candida albicans, Candida krusei, Candida glabrata, Candida parapsilosis, Candida guilliermondii, Aspergillus fumigatus, Rhizopus oryzae, Rhizopus spp., Blastomyces dermatitis, Histoplasma capsulatum, Coccidioides spp., Paecilomyces variotii, Pneumocystis murina, Pneumocystis jiroveci, Histoplasma capsulatum, Aspergillus spp., dimorphic fungi or mucorales fungi.

In some embodiments of the method, the fungal sphingolipid is glucosylceramide (GlcCer).

The present invention yet further provides a method of inhibiting the growth of or killing a fungus in a subject or treating a subject afflicted with a fungal infection comprising administering to the subject an effective amount of the compound of the present invention, or a pharmaceutically acceptable salt or ester thereof, so as to thereby inhibiting the growth of or kill the fungus in the subject or treat the subject afflicted with the fungal infection.

In some embodiments of the method, the method further comprises administering an effective amount of an anti-fungal agent.

In some embodiments of the method, the amount of the compound and the amount of the anti-fungal agent when taken together is more effective to treat the subject than when the anti-fungal agent is administered alone.

In some embodiments of the method, the amount of the compound and the amount of the anti-fungal agent when taken together is effective to reduce a clinical symptom of the fungal infection in the subject.

In some embodiments of the method, the anti-fungal agent is fluconazole, amphotericin B, caspofungin, tunicamycin or aureobasidin A.

In some embodiments of the method, the fungal infection is caused by Candida, Aspergillus, Cryptococcus, Histoplasma, Pneumocystis, Stachybotrys or Mycrorales fungus.

In some embodiments of the method, the fungal infection is caused by Cryptococcus Neoformans.

In some embodiments of the method, the fungal infection is Cryptococcus neoformans cryptococcosis.

In some embodiments of the method, the fungal infection is caused by Sporothrix.

In some embodiments of the method, the fungal infection is caused by S. brasiliensis, S. schenckii, S. globosa, S. mexicana, S. chilensis, S. luriei, and S. pallida.

In some embodiments of the method, the fungal infection is caused by S. brasiliensis.

In some embodiments of the method, the fungal infection is caused by a fungus other than Cryptococcus Neoformans.

In some embodiments of the method, the fungal infection is a fungal infection other than Cryptococcus neoformans cryptococcosis.

In some embodiments of the method, the fungal infection is Aspergillosis, Blastomycosis, Candidiasis, Coccidioidomycosis, Cryptococcus gattii cryptococcosis, Fungal Keratitis, Dermatophytes, Histoplasmosis, Mucormycosis, Pneumocystis pneumonia (PCP), or Sporotrichosis.

In some embodiments of the method, the fungal infection is Sporotrichosis.

In some embodiments of the method, the fungal infection is caused by Cryptococcus gattii, Candida albicans, Candida krusei, Candida glabrata, Candida parapsilosis, Candida guilliermondii, Aspergillus fumigatus, Rhizopus oryzae, Rhizopus spp., Blastomyces dermatitis, Histoplasma capsulatum, Coccidioides spp., Paecilomyces variotii, Pneumocystis murina, Pneumocystis jiroveci, Histoplasma capsulatum, Aspergillus spp., or dimorphic fungi.

In some embodiments of the method, the anti-fungal agent is fluconazole, amphotericin B, caspofungin, tunicamycin or aureobasidin A.

In some embodiments of the method, the fungal infection is caused by Candida, Aspergillus, Cryptococcus, Histoplasma, Pneumocystis, Stachybotrys or Mycrorales fungus.

In some embodiments of the method, the fungal infection is Aspergillosis, Blastomycosis, Candidiasis, Coccidioidomycosis, Cryptococcus gattii cryptococcosis, Fungal Keratitis, Dermatophytes, Histoplasmosis, Mucormycosis, Pneumocystis pneumonia (PCP), or Sporotrichosis.

In some embodiments of the method, the fungal infection is caused by Cryptococcus gattii, Candida albicans, Candida krusei, Candida glabrata, Candida parapsilosis, Candida guilliermondii, Aspergillus fumigatus, Rhizopus oryzae, Rhizopus spp., Blastomyces dermatitis, Histoplasma capsulatum, Coccidioides spp., Paecilomyces variotii, Pneumocystis murina, Pneumocystis jiroveci, Histoplasma capsulatum, or dimorphic fungi.

In some embodiments, the fungal infection is a fungal infection on a plant.

In some embodiments, the fungal infection is an internal fungal infection. In some embodiments, the fungal infection is an invasive fungal infection.

In some embodiments, the fungal infection is a fungal infection of the skin or lung. In some embodiments, the compound has a fungistatic effect on the fungus. In some embodiments, the compound has a fungicidal effect on the fungus. In some embodiments, the compound is administered orally to the subject. In some embodiments, the compound is administered topically to the subject. In some embodiments, the subject is also afflicted with an immunodeficiency disorder. In some embodiments, the subject is also afflicted with human immunodeficiency virus (HIV).

In some embodiments, the antifungal agent is Amphotericin B, Candicidin, Filipin, Hamycin, Natamycin, Nystatin, Rimocidin, Clotrimazole, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Fluconazole, Isavuconazole, Itraconazole, Posaconazole, Ravuconazole, Terconazole, Voriconazole, Abafungin, Amorolfin, Butenafine, Naftifine, Terbinafine, Anidulafungin, Caspofungin, Micafungin, Ciclopirox, Flucytosine, Griseofulvin, Haloprogin, Tolnaftate, or Undecylenic acid.

In some embodiments, a pharmaceutical composition comprising a compound of the present invention and an antifungal agent, and at least one pharmaceutically acceptable carrier for use in treating a fungal infection.

In some embodiments, a pharmaceutical composition comprising an amount of the compound of the present invention for use in treating a subject afflicted with a fungal infection as an add-on therapy or in combination with, or simultaneously, contemporaneously or concomitantly with an anti-fungal agent.

In some embodiments of any of the above methods or uses, the subject is a human. In some embodiments of any of the above methods or uses, the compound and/or anti-fungal agent is orally administered to the subject.

In some embodiments of any of the above methods or uses, the compound and/or anti-fungal agent is topically administered to the subject.

In some embodiments, the fungus or fungal infection has developed resistance to one or more drugs. For example, a drug resistant fungal infection may have developed drug-resistance to an azole antifungal drug, a polyene antifungal drug and/or an echinocandin antifungal drug.

In some embodiments of any of the above methods or uses, the compound targets APL5, COS111, MKK1, and STE2 in the fungus. In some embodiments of any of the above methods or uses, the compound targets at least one of APL5, COS111, MKK1, or STE2 in the fungus. In some embodiments of any of the above methods or uses, the compound disrupts vesicular transport mediate by APL5. In some embodiments of any of the above methods or uses, the fungus carries non-mutated APL5, COS111, MKK1, and STE2. In some embodiments of any of the above methods or uses, the fungus carries at least one of non-mutated APL5, COS111, MKK1, and STE2.

As used herein, a “symptom” associated with a fungal infection includes any clinical or laboratory manifestation associated with the fungal infection and is not limited to what the subject can feel or observe.

As used herein, “treating”, e.g. of a fungal infection, encompasses inducing prevention, inhibition, regression, or stasis of the disease or a symptom or condition associated with the infection.

The contents of U.S. application Ser. No. 16/622,431, now patented as U.S. Pat. No. 11,414,378, are hereby incorporated by reference.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or 3H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano, carbamoyl and aminocarbonyl and aminothiocarbonyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure result.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl, C₃-C₁₂ alkenyl, C₄-C₁₂ alkenyl and so on.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl, C₃-C₁₂ alkynyl, C₄-C₁₂ alkynyl and so on

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “heteroalkyl” includes both branched and straight-chain saturated aliphatic hydrocarbon groups having the specified number of carbon atoms and at least 1 heteroatom within the chain or branch.

As herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzimidazolyl, benzofuranyl, benzofurazanyl, benzopyrazolyl, benzotriazolyl, benzothiophenyl, benzoxazolyl, carbazolyl, carbolinyl, quinolyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, isoxazoline, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, acridinyl, carbazolyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, or pyrrolyl. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heteroarylalkyl” refers to alkyl groups as described above wherein one or more bonds to hydrogen contained therein are replaced by a bond to an heteroaryl group as described above. It is understood that an “heteroarylalkyl” group is connected to a core molecule through a bond from the alkyl group and that the heteroaryl group acts as a substituent on the alkyl group. Examples of heteroarylalkylmoieties include, but are not limited to, —CH₂—(C₅H₄N), —CH₂—CH₂—(C₅H₄N) and the like.

The term “heterocycle” or “heterocyclyl” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. More preferably the ring is three to four-membered and has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, aziridine, azirine, diazirine, oxirane, thiirane, azetidine, oxetane, thetane, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise. In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The terms “substitution”, “substituted” and “substituent” refer to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or pluraly. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure result.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Fumis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobactene. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

Variations on those general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

The following materials and methods are used to test the compounds of the present invention.

### Strains, Media and Reagents

A series of fungal clinical isolates and reference strains were used in this study. This includes Cryptococcus neoformans, Cryptococcus gattii, Candida albicans, Candida krusei, Candida glabrata, Candida parapsilosis, Candida guilliermondii, Aspergillus fumigatus, Rhizopus oryzae, Blastomyces dermatitis, Histoplasma capsulatum, Coccidioides spp. Sporothrix Schenckii, Sporothrix brasiliensis, S. globosa, S. mexicana, S. chilensis, S. luriei, S. pallida, Paecidomyces variotii, Pneumocystis murina, and, Pneumocystis jiroveci. Escherichia coli DH5-α and Pseudomonas aeruginosa were also used. Yeast Peptone Dextrose (YPD), Yeast Nitrogen Base (YNB), Luria Bertani (LB), Roswell Park Memorial Institute (RPMI) or Dulbecco Modified Eagle Medium (DMEM) were purchased from Invitrogen Life Technologies and used as described. Fluconazole, Amphotericin B, Dexamethasone, Cyclophosphamide, Tunicamycin were purchased from Sigma-Aldrich, St Louis, MO. Caspofungin and Posaconazole were obtained from Merck, Rahway, NJ. Voriconazole was obtained from Pfizer, Rey Brook, NY.

### Antifungal Activity Assay.

MICs were determined following the methods of the Clinical and Laboratory Standards Institutes (CLSI) with modifications. Yeast nitrogen base (YNB) medium without amino acid (pH 7.0, 2% glucose) buffered with (4-(2-hydroxyethyl)-1-piperazineethanesulfonic acid) (HEPES) was used for MIC studies in C. neoformans. YNB medium without ammonium sulfate, without amino acid and 1% asparagine (pH 7.0, 2% glucose) was used for MIC studies in Candida strains. RPMI medium (pH 7.0, 2% glucose) was used for MIC studies in A. fumigatus. HEPES was used instead of morpholinepropanesulfonic acid (MOPS), because MOPS was found to inhibit the activity of this kind of compounds. The compounds were serially diluted from 32 to 0.03 μg/mL, in a 96-well plate. The inoculum was prepared as described in the CLSI protocol M27A3 guidelines. The plates were incubated at 37° C. with 5% CO₂ for 24 to 72 h and the optical density was measure at 450 nm. The MICs were determined as the lowest concentration of the compound that inhibited 80% of growth compared to the control.

### Cytotoxicity Assay.

The human cancer cell lines A549 and HepG2 were maintained in Dulbecco's Modified Eagle Medium (DMEM) containing 10% Fetal bovine serum (FBS) and 1% penicillin-streptomycin. At passage 7, 10⁵ cells/well in DMEM containing 10% FBS were transferred into 96-well plates and cultured for 14 h for the cells to adhere to the wells. The compounds were added to the cells at concentrations ranging from 0.03 to 128 μg/mL. The wells without the compound served as controls. The plate was incubated at 37° C. with 5% CO₂. After 24 or 48 h, the supernatant was removed, and 50 μl of 5-mg/mL 3-(4,5-dimethylthiazol-2-yl)-2,5-diphenyltetrazoliumbromide (MTT) solution in Phosphate-buffered saline (PBS) was added to each well. The plates were incubated for an additional 4 h. The formazan crystal formed inside the cell was dissolved by adding 50 μL dimethyl sulfoxide (DMSO). The optical density was measured at 570 nm.

### Time-Kill Assay.

C. neoformans cells from a culture grown overnight were washed in PBS and resuspended in YNB buffered with HEPES at pH 7.4. The cells were counted, and 2×10⁴ cells were incubated with different concentration of the drugs in a final volume of 10 mL with a final concentration of 0.5% DMSO. The tubes were then incubated at 37° C. with 5% CO₂ on a rotary shaker at 200 rpm. Aliquots were taken at time points and diluted, and 100-1 portions were plated onto yeast extract-peptone-dextrose (YPD) plates. YPD plates were incubated in a 30° C. incubator and after 48 h, the numbers of colony forming units (CFU) were counted and recorded.

### In Vitro Killing Assay

From an overnight culture, C. neoformans cells were washed in PBS, resuspended in YNB buffered with HEPES at pH 7.4. Cells were counted and 2×10⁴ cells were incubated with either 1, 2 or 4 μg/ml of compound in a final volume of 10 ml. Tubes were then incubated at 37° C. in the presence of 5% CO₂ on a rotary shaker at 200 rpm. At the illustrated time points, aliquots were taken and diluted and 100 μL was plated onto yeast peptone dextrose (YPD) plates. YPD plates were incubated in a 30° C. incubator and, after 72 hours, colony forming units (CFU) were counted and recorded.

### Intracellular Effect

To assess whether the compound will be effective against intracellular C. neoformans, J774.16 macrophages was incubated with C. neoformans cells at a 1:20 ratio in presence of opsonins (complement and antibody mAb 18B7 against the cryptococcal capsular antigen). After 2 hours of incubation, about 60-80% of macrophages have at least one C. neoformans cell internalized. At this time, wells were washed to remove extracellular fungal cells and fresh DMEM medium without serum and without mAb 18B7 but containing different concentrations of compound was added. Plates were incubated at 37° C. and 5% CO₂. At selected time points, 0, 6, 12 and 24 hours, extracellular cells were collected by washing and plated onto YPD for CFU counting of extracellular cells. Then, macrophages containing C. neoformans were lysed, collected and serial dilutions were plated onto YPD for CFU counting of intracellular fungal cells.

### Synergistic Assay

Synergistic activity was assayed by calculating the fractional inhibitory index (FIC) as previously described (Del Poeta, M. et al. 2000). Briefly, in a 96 well plate, the compound was serially diluted from 16 to 0.015 μg/ml (11 dilutions) whereas drug B (e.g., either Fluconazole, Amphotericin B, Caspofungin, or Tunicamycin) was serially diluted from 12 to 0.19 μg/ml, 5 to 0.078 μg/ml, 70 to 1.09 μg/ml, and 6 to 0.09 μg/ml (7 dilutions), respectively. The FIC was defined as: [MIC combined/MIC Drug A alone]+[MIC combined/MIC Drug B alone].

### Resistance Assay

To see whether incubation with the drugs will induce resistance, C. neoformans cells were passaged daily in sub-MIC drug concentrations. Briefly, from an overnight culture, C. neoformans cells were washed with PBS, resuspended in YNB buffered with HEPES at pH 7.4 and counted. Then, 10⁶ cells were incubated with 0.5, 0.25 or 0.125 μg/ml of compound or 0.15, 0.075 and 0.037 μg/ml of compound in 1 ml final volume. Tubes without the drug served as negative control. Tubes with Fluconazole (0.5, 1 and 2 μg/ml) served as positive control. The cells were grown at 37° C. in the presence on 5% CO₂ on a rotary shaker at 200 rpm. Every 24 hours, the cells were pelleted by centrifugation, washed with PBS, and resuspended in YNB, and 10⁶ cells were transferred into a fresh drug tube and incubated as above. These daily passages were continued for 15 days. Cell aliquots were collected on day 0 (before any drug exposure), 5, 10, 15, and MIC was determined using the microbroth dilution assay as described above.

### Animal Studies for Cryptococcosis

For survival studies, 4-week old CBA/J female mice (Jackson Laboratory, Bar Harbor, ME) were used. Ten mice per treatment or control group were used. Mice were infected by nasal inoculation of 20 μL containing 5×10⁵ cells of C. neoformans H99 strain. Treated mice received an intraperitoneal injection of 1.2 mg/kg/day of compound in 100 μL final volume of PBS containing 0.4% DMSO. Untreated mice, received 100 μL of PBS/0.4% DMSO. Mice were feed ad-libitum and monitored closely for sign of discomfort and meningitis. Mice showing abnormal gait, lethargic, tremor, significant loss of body weight or inability to reach water or food were sacrificed and survival counted from that day. At the end of the survival study, tissue burden culture was performed in mice that survived the infection. Mice were sacrificed, and their organs were extracted, and homogenized in 10 ml sterile PBS using a homogenizer (Stomacher80, Cole-Parmer, Vernon Hills, IL). Organ homogenates were serially diluted 1:10 in PBS and 100 μL was plated on YPD agar plates and incubated at 30° C. for 72 hours for CFU count. For histopathology, extracted organs were fixed in 10% formalin before paraffin sectioning and staining with either Hematoxylin-Eosin or Mucicarmine. Images were taken at 40× in a Zeiss Axio Observer in brightfield mode.

### Animal Studies for Pneumocystosis

For survival studies, C3H/HeN mice ordered from the National Cancer Institute (Bethesda, MD) were used. Mice were infected with P. murina pneumonia through exposure to mice with a fulminant P. murina infection (seed mice). These mice were immune suppressed by the addition of dexamethasone at 4 mg/liter to the drinking water. Sulfuric acid at 1 ml/liter was also added to the drinking water for disinfection. The seed mice are rotated within the cages for 2 weeks and then removed. After the mice had developed a moderate infection level (approximately 5 weeks), they were divided into a negative control group (control steroid), positive control group (trimethoprim/sulfamethoxazole) and treatment groups (compound). Twelve mice were used in each group. Compound was administered intraperitoneally or by oral gavage on a mg/kg/day basis for up to 3 weeks. The dose, route, and frequency of administration varied depending on the agent being tested. At the end of the treatment, mice were sacrificed and processed for analysis. Slides were made from the lung homogenates at different dilutions and stained with Diff-Quik to quantify the trophic forms and Cresyl Echt violet to quantify the asci. Additional group of mice were selectively depleted of their CD4+ lymphocytes by antibody treatment with 300 μg of GK 1.5 antibody (Biovest International, Minneapolis, MN) administered intraperitoneally 3 times on days 1, 3, and 7. After this initial treatment, the mice were infected by exposure to P. murina infected mice. Mice then were treated with 100 μg of GK 1.5 antibody intraperitoneally once a week for 6 weeks. Mice were then treated with 1.25 or 12.5 mg/kg/day of 1 for 14 days while continuing the GK 1.5 treatment. Control mice received vehicle.

### Animal Studies for Candidiasis

For survival studies, 8-week old CBA/J female mice (Jackson Laboratory) were used. Eight mice per treatment or control group were used. Mice were infected by intravenous inoculation of 100 μL containing 1×10⁵ cells of Candida albicans SC-5314 strain. Treated mice received an intraperitoneal injection of 1.2 mg/kg/day of compound in 100 μL final volume of PBS containing 0.4% DMSO. Untreated mice, received 100 μL of PBS/0.4% DMSO. Mice were feed ad-libitum and monitored closely for sign of discomfort. At the end of the survival study, tissue burden culture was performed in mice that survived the infection. Mice were sacrificed and their organs were extracted and homogenized in 10 ml sterile PBS using homogenizer. Organ homogenates were diluted 10 times in PBS, and 100 μL was plated on YPD agar plates and incubated at 30° C. for 72 hours for CFU count.

### Toxicity

In vitro. The murine macrophage cell line J774.16 was maintained in DMEM containing 10% FBS and 1% Pen-strep. At passage #7, 10⁵ cells/well in DMEM containing 10% FBS was transferred into 96 well plates and cultured for 14 hours for the cells to adhere to the wells. The compound was added to the cells at concentration ranging from 0.1 to 100 μg/ml. The wells without the drug served as control. The plate was incubated at 37° C. in the presence of 5% CO₂. After 12 or 24 hours, the supernatant was removed and 50 μL of 5 mg/ml of 3-(4,5-dimethylthiazol-2-yl)-2,5-diphenyltetrazolium bromide (MIT) solution in PBS was added to each well and plates incubated for 4 additional 4 hours. The formazan crystal formed inside the cell was dissolved by adding 50 μL of isopropanol containing 0.1 N HCL. The optical density was measured at 570 nm.

To determine whether the compound's toxicity was enhanced by corticosteroids, a separate set of 1774.16 cells were incubated with 10 or 100 μg/ml of Dexamethasone alone or combined with either 1, 5 and 10 μg/ml of compound After 24 hours, the MTT assay was performed as described above.

### Lipid Mass Spectrometry

For lipid analysis by mass spectrometry, fungal cells (C. neoformans or C. albicans) were grown in YNB and incubated with compound as explained for the in vivo labeling (except that tritiated palmitate was not added), for 6 hrs. Samples without drug were included as control. Before lipid extraction, lipid internal standards (C17 ceramide and C17 sphingosine) were added. Lipids were then extracted following the methods of Mandala and Bligh and Dyer and one fourth of the sample was aliquoted for determination of the inorganic phosphate. The remainder of the sample was subjected to base hydrolysis and then analyzed using LC/MS. Results were normalized with the inorganic phosphate levels.

### In Vitro Activity of Gcs1

For the in vitro Gcs1 assay, C. neoformans wild-type (WT) or the Δgcs1 cells were grown in YPD broth overnight at 30° C. in a shaker incubator. Cells were washed with sterile water and then lysed by bead beating in presence of glass bead and protease cocktail inhibitor, as described (Liberto, C. et al. 2001). Next, 800 μg of cell lysate was incubated with 0.3 mM C16 ceramide (C16-R—OH) and in the presence or absence of compound. The mixture was subjected to 3 cycles of sonication (20 sec) and vortexing (5 sec). Next, 8 μM of radiolabelled UDP-¹⁴C-Glucose (American Radiolabeled Chemical) was added and, after brief vortexing, the tubes were incubated at 37° C. for 45 min. The reaction was stopped by adding 0.9 ml of 0.45% NaCl solution containing chloroform:methanol 2:1. The organic phase was collected in a glass tube and flushed with nitrogen. The sample was dried and resuspended in chloroform:methanol 1:1. Sample was then loaded on a TLC plate using by chloroform:methanol:water as the mobile phase.

### C6-NBD-Ceramide Staining

The Golgi apparatus of C. neoformans and C. albicans was stained with C6-NBD-ceramide using a previously described protocol (Kmetzsch, L. et al. 2011), based on the property that this fluorescent lipid accumulates at the Golgi of either living or fixed cells (Pagano R. E. et al. 1989). Control or compound-treated (4 μg/ml) yeast cells were fixed with 4% paraformaldehyde in PBS. Cell suspensions were then washed with the same buffer and incubated with C6-NBD-ceramide (20 mM) for 16 h at 4° C. The cells were then incubated with bovine serum albumin (BSA, 1%) at 4° C. for 1 h to remove the excess of C6-NBD-ceramide. After washing with PBS, the cells were incubated with 10 □g/ml DAPI (Sigma-Aldrich, St. Louis, USA) for 30 min at room temperature. The cells were washed again with PBS and stained cell suspensions were mounted over glass slides as described above and analyzed under an Axioplan 2 (Zeiss, Germany).

### Statistical Analysis

Statistical analysis for survival studies was performed using Student-Newman-Keuls t test for multiple comparisons using INSTAT. Statistical analysis for tissue burden and for trophic form and asci counts was performed using the analysis of variance (ANOVA). Additional statistic was performed using Student t test.

### Comparison Studies

For survival studies, 4-week old CBA/J female mice (Jackson Laboratory, Bar Harbor, ME) were used. Total of forty mice were infected by tail vein injection of 200 μL containing 10⁵ cells of C. neoformans H99 and were randomly separated into 5 groups (8 mice per group). Treatment started within 2 hours of infection. The treated mice received an intraperitoneal injection of 1.2 mg/kg/day of compound and amphotericin B or 10 mg/kg/day of fluconazole in 100 μL final volume of PBS containing 0.4% DMSO. Untreated mice, received 100 μL of PBS/0.4% DMSO. Mice were fed ad-libitum and monitored closely for sign of discomfort and meningitis. Mice showing abnormal gait, lethargy, tremor, significant loss of body weight, or inability to reach water or food were sacrificed and survival was counted until that day.

### Sample Preparation for Transmission Electron Microscopy (TEM)

Sample preparation for Transmission electron Microscopy (TEM) was performed similar to the methods of Heung (Heung et al. 2005) with minor modifications. Briefly, C. neoformans (H99) were grown in YNB (pH=7.4) at 37° C. and 5% CO₂ and treated for 6 hours with compound (4 μg/mL), non-treated cells were also included as control. The cells were pelleted at 3000 rpm (1700 g) and fixed with 2% EM glutaraldehyde in PBS solution for 1 hour. Samples were then washed in PBS, placed in 1% osmium tetroxide in 0.1 M PBS, dehydrated in a graded series of ethyl alcohol and embedded in Embed812 resin. Ultrathin sections of 80 nm were cut with a Leica EM UC7 ultramicrotome (Leica Microsystems Inc., Buffalo Grove, IL) and placed on uncoated mesh copper grids. Sections were then counterstained with uranyl acetate and lead citrate and viewed with a FEI Tecnail2 BioTwinG2 electron microscope (FEI, Hillsboro, Oregon) Transmission Electron Microscope (TEM). Digital images were acquired with an AMT XR-60 CCD Digital Camera system.

TABLE 1
Broad Spectrum In Vitro Antifungal Activity of New Ethynylacylhydrazones.

| | | | MIC₈₀ (μg/mL) | | |
|---|---|---|---|---|---|
| Entry | Codes | Structures | C. neoformans | C. albicans | A. fumigatus |
| 1 | SB-AF-10-23 | 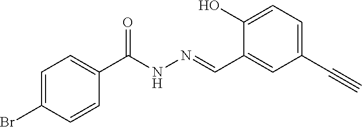 [SMILES: C#Cc1ccc(O)c(/C=N/NC(=O)c2ccc(Br)cc2)c1] | 0.007 | 0.06 | 2 |
| 2 | SB-AF-08-23 | 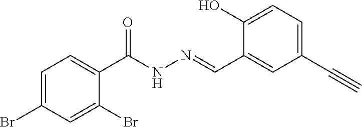 [SMILES: C#Cc1ccc(O)c(/C=N/NC(=O)c2ccc(Br)cc2Br)c1] | 0.03 | ≤0.01 | 2 |
| 3 | SB-AF-13-23 | 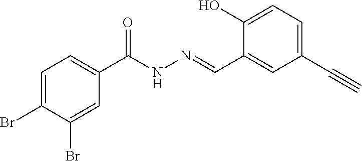 [SMILES: C#Cc1ccc(O)c(/C=N/NC(=O)c2ccc(Br)c(Br)c2)c1] | 0.06 | 0.5 | 2 |
| 4 | SB-AF-10-27 | 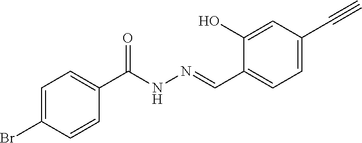 [SMILES: C#Cc1ccc(/C=N/NC(=O)c2ccc(Br)cc2)c(O)c1] | 0.03 | 4 | >16 |
| 5 | SB-AF-08-27 | 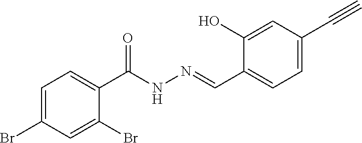 [SMILES: C#Cc1ccc(/C=N/NC(=O)c2ccc(Br)cc2Br)c(O)c1] | 0.03 | 0.12 | 0.5 |
| 6 | SB-AF-13-27 | 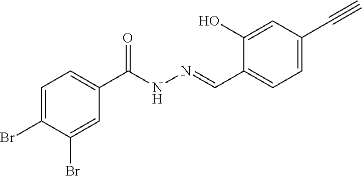 [SMILES: C#Cc1ccc(/C=N/NC(=O)c2ccc(Br)c(Br)c2)c(O)c1] | 0.007 | 8 | >16 |
| 7 | SB-AF-12-27 | 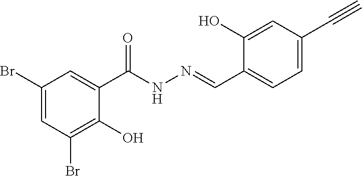 [SMILES: C#Cc1ccc(/C=N/NC(=O)c2cc(Br)cc(Br)c2O)c(O)c1] | 0.03 | 2 | >16 |
| 8 | SB-AF-36-27 | 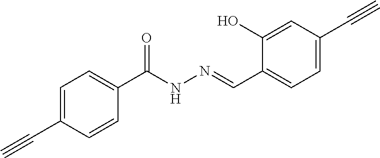 [SMILES: C#Cc1ccc(C(=O)N/N=C/c2ccc(C#C)cc2O)cc1] | 0.007 | 16 | >16 |
| 9 | SB-AF-46-27 | 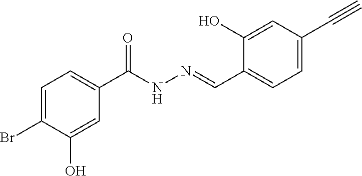 [SMILES: C#Cc1ccc(/C=N/NC(=O)c2ccc(Br)c(O)c2)c(O)c1] | 1 | >16 | >32 |
| 10 | SB-AF-10-28 | 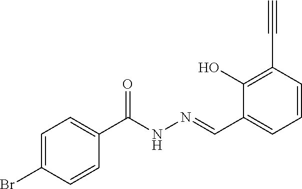 [SMILES: C#Cc1cccc(/C=N/NC(=O)c2ccc(Br)cc2)c1O] | 0.007 | 2 | 4 |
| 11 | SB-AF-13-28 | 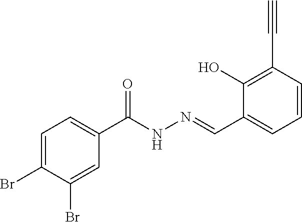 [SMILES: C#Cc1cccc(/C=N/NC(=O)c2ccc(Br)c(Br)c2)c1O] | 0.015 | 4 | 1 |
| 12 | SB-AF-08-29 | 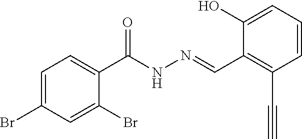 [SMILES: C#Cc1cccc(O)c1/C=N/NC(=O)c1ccc(Br)cc1Br] | 0.031 | 0.5 | >32 |
| 13 | SB-AF-13-29 | 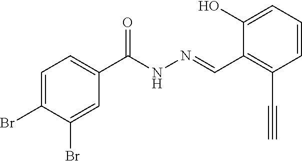 [SMILES: C#Cc1cccc(O)c1/C=N/NC(=O)c1ccc(Br)c(Br)c1] | 0.06 | 16 | 8 |
| 14 | SB-AF-10-29 | 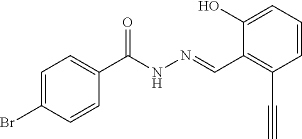 [SMILES: C#Cc1cccc(O)c1/C=N/NC(=O)c1ccc(Br)cc1] | 0.125 | 8 | >32 |
| 15 | SB-AF-41-27 | 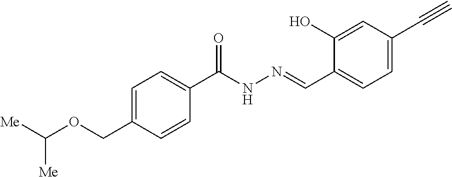 [SMILES: C#Cc1ccc(/C=N/NC(=O)c2ccc(COC(C)C)cc2)c(O)c1] | ≤0.015 | >16 | — |
| 16 | SB-AF-44-27 | 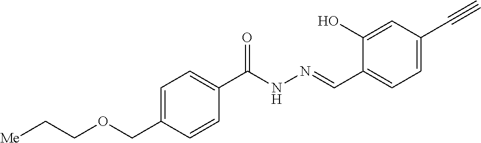 [SMILES: C#Cc1ccc(/C=N/NC(=O)c2ccc(COCCC)cc2)c(O)c1] | 0.25 | >16 | — |
| 17 | SB-AF-40-27 | 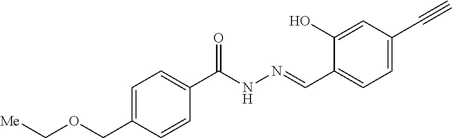 [SMILES: C#Cc1ccc(/C=N/NC(=O)c2ccc(COCC)cc2)c(O)c1] | 0.031 | 8 | — |
| 18 | SB-AF-12-23 | 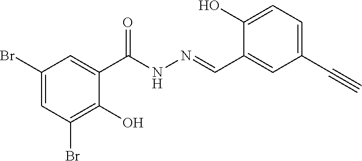 [SMILES: C#Cc1ccc(O)c(/C=N/NC(=O)c2cc(Br)cc(Br)c2O)c1] | 0.03 | 0.12 | 4 |
| 19 | SB-AF-39-23 | 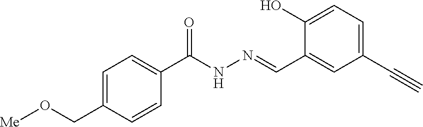 [SMILES: C#Cc1ccc(O)c(/C=N/NC(=O)c2ccc(COC)cc2)c1] | 0.25 | — | — |
| 20 | SB-AF-25-23 | 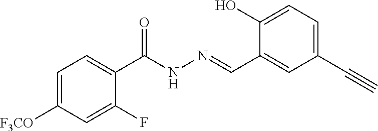 [SMILES: C#Cc1ccc(O)c(/C=N/NC(=O)c2ccc(OC(F)(F)F)cc2F)c1] | 4 | — | — |
| 21 | SB-AF-43-23 | 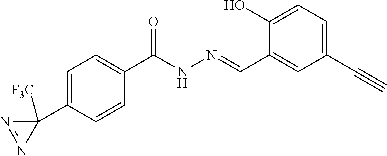 [SMILES: C#Cc1ccc(O)c(/C=N/NC(=O)c2ccc(C3(C(F)(F)F)N=N3)cc2)c1] | 0.007 | 1 | 1 |

TABLE 2
Antifungal activities of new acylhydrazones against C. neoformans

| | | | MIC₈₀ |
|---|---|---|---|
| Compound | Ring A | Ring B | (mg/mL) |
| SB-AF-08-24 | 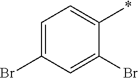 [SMILES: *c1ccc(Br)cc1Br] | 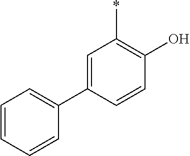 [SMILES: *c1cc(-c2ccccc2)ccc1O] | 0.06 |
| SB-AF-13-24 | 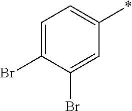 [SMILES: *c1ccc(Br)c(Br)c1] | 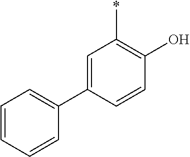 [SMILES: *c1cc(-c2ccccc2)ccc1O] | 0.06 |
| SB-AF-40-17 | 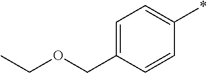 [SMILES: *c1ccc(COCC)cc1] | 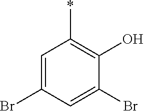 [SMILES: *c1cc(Br)cc(Br)c1O] | 0.007 |
| SB-AF-40-13 | 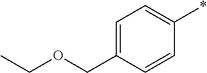 [SMILES: *c1ccc(COCC)cc1] | 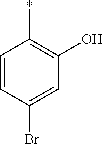 [SMILES: *c1ccc(Br)cc1O] | 0.06 |
| SB-AF-44-17 | 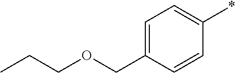 [SMILES: *c1ccc(COCCC)cc1] | 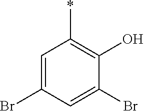 [SMILES: *c1cc(Br)cc(Br)c1O] | 0.007 |
| SB-AF-44-15 | 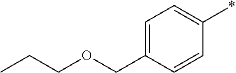 [SMILES: *c1ccc(COCCC)cc1] | 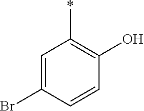 [SMILES: *c1cc(Br)ccc1O] | 0.007 |
| SB-AF-41-17 | 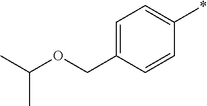 [SMILES: *c1ccc(COC(C)C)cc1] | 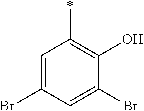 [SMILES: *c1cc(Br)cc(Br)c1O] | 0.03 |
| SB-AF-41-15 | 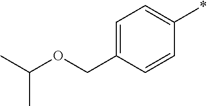 [SMILES: *c1ccc(COC(C)C)cc1] | 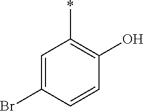 [SMILES: *c1cc(Br)ccc1O] | 0.06 |
| SB-AF-41-13 | 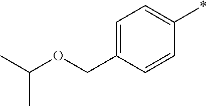 [SMILES: *c1ccc(COC(C)C)cc1] | 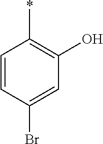 [SMILES: *c1ccc(Br)cc1O] | 0.06 |

### Example 2. Administration of the Compound

An amount of the compound of the present invention is administered to a subject afflicted with a fungal infection. The amount of the compound is effective to treat the subject.

An amount of the compound of the present invention is administered to a subject afflicted with a fungal infection. The amount of the compound is effective to treat the subject by inhibiting sphingolipid synthesis in the fungus without substantially inhibiting sphingolipid synthesis in the subject.

An amount of the compound of the present invention in combination with an anti-fungal agent are administered to a subject afflicted with a fungal infection. The amount of the compound and the agent are effective to treat the subject.

### Example 3: Assessment of Efficacy of Compound as Add-On Therapy to Anti-Fungal Agents

The add-on therapy provides a synergistic effect, and allows for lower doses with reduced side effects and resistance.

Periodic administration of the compound of the present invention as an add-on therapy for a subject afflicted with a fungal infection who is already receiving treatment with an anti-fungal agent provides a clinically meaningful advantage and is more effective (provides at least an additive effect or more than an additive effect) in treating the subject than when the anti-fungal agent is administered alone (at the same dose).

Periodic administration an anti-fungal agent as an add-on therapy for a human patient afflicted with a fungal infection who is already receiving a compound of the present invention provides a clinically meaningful advantage and is more effective (provides at least an additive effect or more than an additive effect) in treating the subject than when the compound is administered alone (at the same dose).

The add-on therapies also provide efficacy (provides at least an additive effect or more than an additive effect) in treating the subject without undue adverse side effects or affecting the safety of the treatment. As compared to when each agent is administered alone:
1. The add-on therapy is more effective (provides an additive effect or more than an additive effect) in killing the fungus; and/or
2. The add-on therapy is more effective (provides an additive effect or more than an additive effect) in slowing the growth of the fungus.

### Example 4: Synthesis and Characterization

Chemical Synthesis and Characterization of Acylhydrazones of this Invention

2-Hydroxy-5-((trimethylsilyl)ethynyl)benzaldehyde (2); A 100 ml two neck round bottom flask was charged with 5-bromosalicylaldehyde, 1 and dry triethylamine under an atmosphere of N₂ and the solution was stirred for five minutes at room temperature. Following, PdCl₂(PPh₃)₂ and CuI were added, and the solution was degassed. Under a slow flow of nitrogen ethynyltrimethylsilane was added quickly and the reaction mixture was stirred at 80° C. for 3 hours. The reaction mixture was then slowly cooled to room temperature; later dry THF was added to the reaction mixture and stirred at room temperature for 1 hour. The mixture was concentrated in vacuum, diluted with CH₂Cl₂, washed with water. The aqueous phase was extracted with CH₂Cl₂, the organic layers were combined and dried over anhydrous Mg₂SO₄. The solvent was evaporated and the remaining solid was purified by gradient flash chromatography on silica gel to yield the product as light yellow crystals, (26-50% yield); ¹H NMR (700 MHz, CDCl₃) δ 0.27 (s, 9H), 6.9 (d, 1H, J=8.9 Hz), 7.61 (dd, 1H, J=8.7 Hz), 7.73 (d, 1H, J=2.0 Hz), 9.87 (s, 1H), 11.12 (s, 1H); ¹³C NMR (125 MHz, CDCl₃) δ 81.8, 114.0, 118.1, 120.3, 137.5, 140.2, 161.7, 196.0 These data are consistent with the literature values.[¹]

The same procedure was used for the synthesis of 5, 8 and 11.

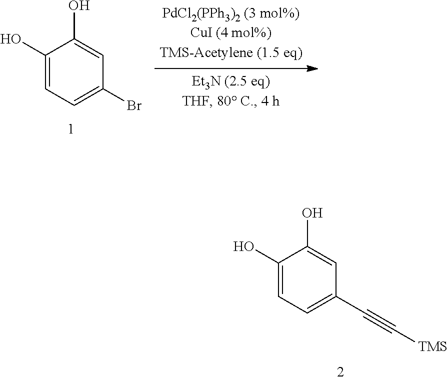 [SMILES: Oc1ccc(Br)cc1O ; C[Si](C)(C)C#Cc1ccc(O)c(O)c1]

5-Ethynyl-2-hydroxybenzaldehyde (3); To a solution of 2-Hydroxy-5-(2-(trimethylsilyl)ethynyl)benzaldehyde, 2 in dry THF, a freshly prepared solution of TBAF in dry THF was added. The reaction mixture was stirred at room temperature for 2 hours, and water was added. The mixture was extracted with Et₂O (2×40 mL), the organic layers were combined and dried over anhydrous Mg₂SO₄, the solution was filtered and concentrated in vacuum. Purification was performed by flash chromatography on silica gel (Hexane/Ethyl Acetate 20:1) and yielded yellow crystalline solid, 74% yield; Rf=0.32 (Hexane:Ethyl Acetate, 20:1). ¹H NMR (700 MHz, CDCl₃) δ 3.06 (s, 1H), 6.9 (d, 1H, J=8.9 Hz), 7.64 (dd, 1H, J=8.7 Hz), 7.74 (d, 1H, J=2.0 Hz), 9.89 (s, 1H), 11.15 (s, 1H). ¹³C NMR (125 MHz, CDCl₃) δ 93.8, 103.1, 115.1, 117.9, 120.3, 137.5, 140.1, 161.5, 196.0 These data are consistent with the literature values.^{[1}]

The same procedure was used for the synthesis of 6, 9 and 12.

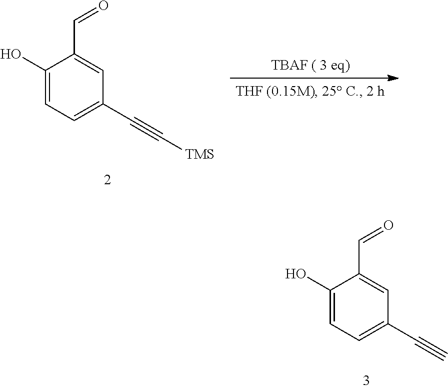

2-Hydroxy-4-((trimethylsilyl)ethynyl)benzaldehyde (5); Yellow solid, (1.113 g, 63% yield); ¹H NMR (400 MHz, CDCl₃) δ 0.26 (s, 9H), 7.06-7.08 (m, 2H), 7.48 (d, 1H, J=2.0 Hz), 9.87 (s, 1H), 10.98 (s, 1H); These data are consistent with the literature values.[²]

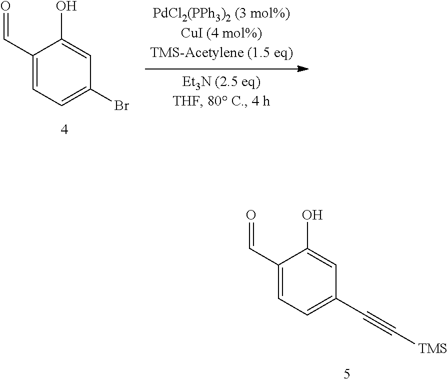 [SMILES: O=Cc1ccc(Br)cc1O ; C[Si](C)(C)C#Cc1ccc(C=O)c(O)c1]

4-Ethynyl-2-hydroxybenzaldehyde (6); Brown solid, (0.755 g, 89% yield); ¹H NMR (700 MHz, CDCl₃) δ 3.28 (s, 1H), 7.11 (m, 2H), 7.52 (d, 1H, J=2.0 Hz), 9.89 (s, 1H), 11.01 (s, 1H); These data are consistent with the literature values.[²]

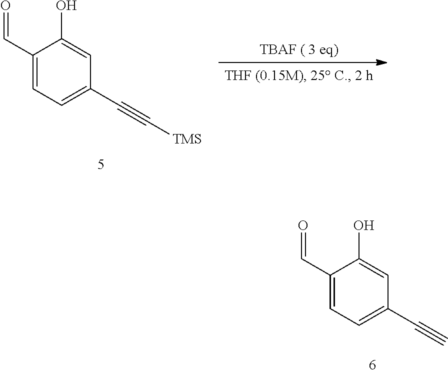

2-Hydroxy-3-((trimethylsilyl)ethynyl)benzaldehyde (3); Light yellow solid, (0.998 g, 60% yield); Rf=0.44 (Hexane:Ethyl Acetate, 20:1); ¹H NMR (700 MHz, CDCl₃) δ 0.28 (s, 9H), 6.96 (d, 1H, J=8.9 Hz), 7.60 (dd, 1H, J=8.7 Hz), 7.69 (d, 1H, J=2.0 Hz), 9.89 (s, 1H), 11.45 (s, 1H); ¹³C NMR (125 MHz, CDCl₃) δ 81.8, 114.0, 118.1, 120.3, 137.5, 140.2, 161.7, 196.0 These data are consistent with the literature values. [³]

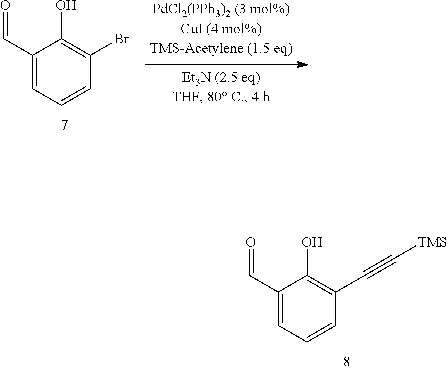 [SMILES: O=Cc1cccc(Br)c1O ; C[Si](C)(C)C#Cc1cccc(C=O)c1O]

3-Ethynyl-2-hydroxybenzaldehyde (5); Yellow solid (0.475 g, 75% yield); Rf=0.30 (Hexane:Ethyl Acetate, 20:1). 1H NMR (500 MHz, CDCl₃) δ 11.51 (s, 1H), 9.89 (s, 1H), 7.69 (d, J=7.4 Hz, 1H), 7.57 (d, J=7.7 Hz, 1H), 7.00 (t, J=7.7 Hz, 1H), 3.38 (s, 1H). ¹³C NMR (125 MHz, CDCl₃) δ 81.8, 114.0, 118.1, 120.3, 137.5, 140.2, 161.7, 196.0 These data are consistent with the literature values. [³]

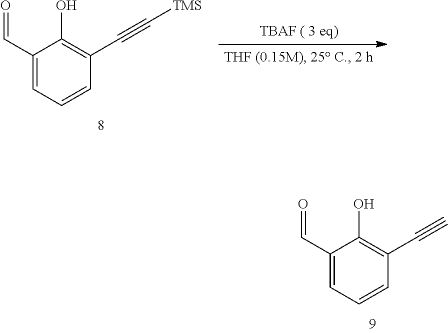

2-Hydroxy-6-((trimethylsilyl)ethynyl)benzaldehyde (4) Pale yellow solid, (0.77 g, 71% yield); Rf=0.43 (Hexane:Ethyl Acetate, 20:1). ¹H NMR (700 MHz, CDCl₃) δ 0.27 (s, 9H), 6.9 (d, 1H, J=8.9 Hz), 7.61 (dd, 1H, J=8.7 Hz), 7.73 (d, 1H, J=2.0 Hz), 9.87 (s, 1H), 11.12 (s, 1H); ¹³C NMR (125 MHz, CDCl₃) δ 81.8, 114.0, 118.1, 120.3, 137.5, 140.2, 161.7, 196.0 These data are consistent with the literature values'.[3]

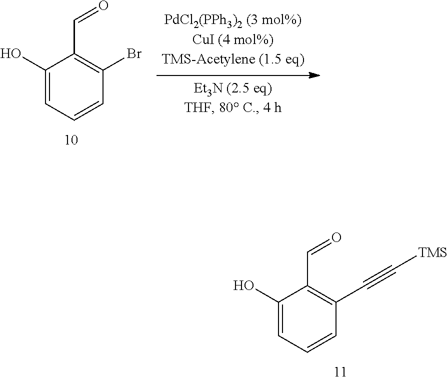 [SMILES: O=Cc1c(O)cccc1Br ; C[Si](C)(C)C#Cc1cccc(O)c1C=O]

2-Ethynyl-6-hydroxybenzaldehyde (6) Yellow solid, (0.400 g, 79% yield); Rf=0.34 (Hexane:Ethyl Acetate, 20:1). ¹H NMR (500 MHz, CDCl₃) δ 11.67 (s, 1H), 10.45 (s, 1H), 7.45 (t, J=8.0 Hz, 1H), 7.13 (d, J=7.5 Hz, 1H), 6.99 (d, J=8.5 Hz, 1H), 3.44 (s, 1H). These data are consistent with the literature values. [³]

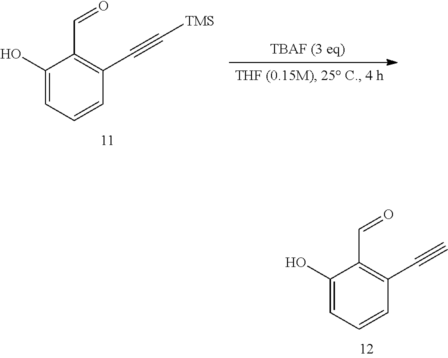

4-Bromo-N′-(5-ethynyl-2-hydroxybenzylidene) benzohydrazide (SB-AF-10-23). To a solution of 4-bromobenzohydrazide and 5-ethynyl-2-hydroxybenzaldehyde, 3 (1.05 eqv) in methanol, catalytic amount of glacial acetic acid was added. The reaction mixture was stirred at room temperature overnight. Addition of water to the reaction mixture resulted in precipitation of the product, which was filtered, washed with water, and dried to give pure product as yellow solid (72% yield); Rf=0.34 (Hexanes:Ethyl Acetate, 2:1); mp=188-190° C.; ¹H NMR (700 MHz, Acetone-d₆). δ 4.06 (s, 1H), 6.9 (d, 1H, 10 Hz), 7.41 (d, 1H, 10 Hz), 7.76 (m, 3H, 10 Hz), 7.90 (d, 2H, J=2.12 Hz), 8.63 (s, 1H), 11.52 (s, 1H), 12.25 (s, 1H)¹³C NMR (175 MHz, Acetone-d₆) δ 79.6, 80.1, 113.0, 117.5, 119.7, 126.3, 129.5, 130.1, 132.6, 134.6, 135.2, 138.8, 147.8, 158.8, 162.4; HRMS (TOF) m/z calcd for C₁₆H₁₁BrN₂O₂H⁺: 343.0077, found: 343.0084 (Δ=−2.13 ppm).

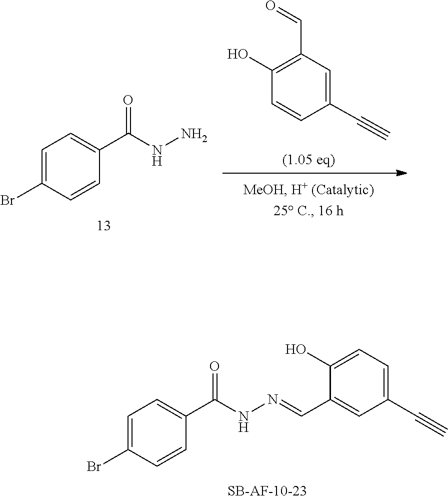 [SMILES: NNC(=O)c1ccc(Br)cc1 ; C#Cc1ccc(O)c(C=O)c1 ; C#Cc1ccc(O)c(/C=N/NC(=O)c2ccc(Br)cc2)c1]

The same procedure was used for the synthesis of SB-AF-08-23, SB-AF-13-23, SB-AF-10-27, SB-AF-08-27, SB-AF-13-27, SB-AF-12-27, SB-AF-36-27, SB-AF-46-27, SB-AF-10-28, SB-AF-13-28, SB-AF-08-29, SB-AF-13-29, SB-AF-10-29, SB-AF-41-27, SB-AF-44-27, SB-AF-40-27, SB-AF-12-23, SB-AF-39-23, SB-AF-25-23, SB-AF-43-23.

2,4-Dibromo-N′-(5-ethynyl-2-hydroxybenzylidene)benzohydrazide (SB-AF-08-23); White solid (0.05 g, 73% yield); Rf=0.36 (Hexanes:Ethyl Acetate, 2:1); mp 226-228° C.; ¹H NMR (400 MHz, DMSO-d₆) δ 3.96 (s, 1H, 37%), 4.02 (s, 1H, 63%), 6.81 (d, 1H, J=8.48 Hz, 35%), 6.92 (d, 1H, J=8.52 Hz, 65%), 7.28 (dd, 1H, J=8.44 Hz, 2.02 Hz, 35%), 7.34 (d, 1H, J=1.92 Hz, 37%), 7.37-7.40 (m, 1H, 100%) 7.53 (d, 1H, J=8.16 Hz, 63%), 7.68-7.75 (m, 2H, 100%, 66%), 7.98-8.01 (m, 1H, 100%), 8.27 (s, 1H, 35%), 8.45 (s, 1H, 65%), 10.32 (s, 1H, 30%), 11.25 (s, 1H, 70%), 12.17 (s, 1H, 100%); ¹³C NMR (100 MHz, DMSO-d₆) δ 79.3, 83.2, 83.3, 112.7, 112.9, 116.9, 117.1, 119.5, 119.8, 120.0, 120.8, 122.9, 124.0, 130.3, 130.91, 130.98, 131.1, 132.1, 134.4, 134.7, 134.96, 135.03, 136.2, 137.4, 142.5, 146.6, 157.1, 157.8, 162.7, 168.4; HRMS (TOF) m/z calcd for C₁₆H₁₀Br₂N₂O₂H⁺: 420.9182, found: 420.9185 (Δ=−0.68 ppm).

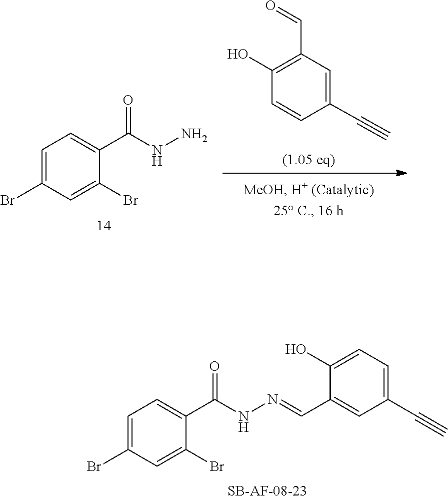 [SMILES: NNC(=O)c1ccc(Br)cc1Br ; C#Cc1ccc(O)c(C=O)c1 ; C#Cc1ccc(O)c(/C=N/NC(=O)c2ccc(Br)cc2Br)c1]

3,4-Dibromo-N′-(5-ethynyl-2-hydroxybenzylidene)benzohydrazide (SB-AF-13-23); Brown solid (70% yield); Rf=0.35 (Hexanes:Ethyl Acetate, 2:1); mp 209-211° C.; ¹H NMR (700 MHz, DMSO-d₆) δ 4.05 (s, 1H), 6.93 (d, J=8.5 Hz, 1H), 7.39 (dd, J=8.5, 2.0 Hz, 1H), 7.76 (d, J=1.9 Hz, 1H), 7.85 (dd, J=8.3, 1.9 Hz, 1H), 7.94 (d, J=8.3 Hz, 1H), 8.29 (d, J=1.9 Hz, 1H), 8.61 (s, 1H), 11.42 (s, 1H), 12.27 (s, 1H); ¹³C NMR (175 MHz, DMSO-d₆) δ 23 79.4, 83.3, 112.8, 117.2, 119.5, 124.4, 128.3, 128.7, 132.3, 132.7, 133.8, 134.3, 134.9, 147.0, 157.9, 160.9. HRMS (TOF) m/z calcd for C₁₆H₁₀Br₂N₂O₂H⁺: 420.9182, found: 420.9186 (Δ=−1.01 ppm).

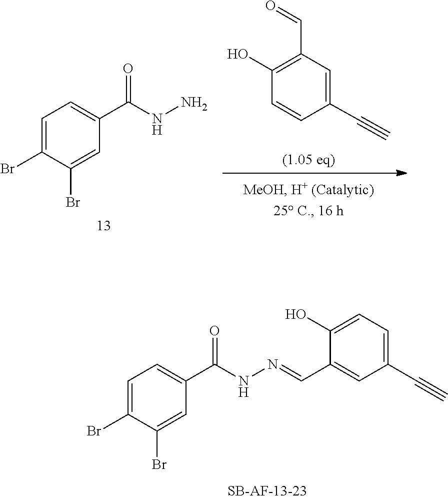 [SMILES: NNC(=O)c1ccc(Br)c(Br)c1 ; C#Cc1ccc(O)c(C=O)c1 ; C#Cc1ccc(O)c(/C=N/NC(=O)c2ccc(Br)c(Br)c2)c1]

4-Bromo-N′-(4-ethynyl-2-hydroxybenzylidene)benzohydrazide (SB-AF-10-27); Yellow solid (83% yield); Rf=0.32 (Hexanes:Ethyl Acetate, 2:1); mp=220° C.; ¹H NMR (500 MHz, Acetone-d₆) δ 3.79 (s, 1H), 7.06 (d, 2H, J=10 Hz), 7.41 (d, 1H, J=10 Hz), 7.76 (d, 2H, J=10 Hz), 7.96 (d, 2H, J=2.12 Hz), 8.61 (s, 1H), 11.54 (s, 1H), 11.74 (s, 1H). ¹³C NMR (175 MHz, DMSO-d₆) δ 82.7, 83.6, 119.6, 120.4, 123.3, 124.6, 126.3, 129.4, 130.2, 132.0, 147.2, 157.4, 162.4. HRMS (TOF) m/z calcd for C₁₆H₁₁BrN₂O₂H⁺: 343.0077, found: 343.0077 (Δ=−0.17 ppm).

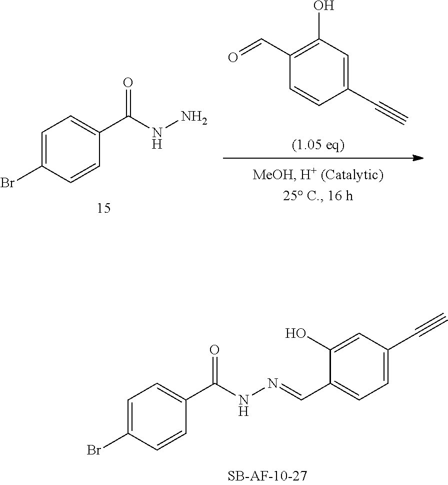 [SMILES: NNC(=O)c1ccc(Br)cc1 ; C#Cc1ccc(C=O)c(O)c1 ; C#Cc1ccc(/C=N/NC(=O)c2ccc(Br)cc2)c(O)c1]

2,4-Dibromo-N′-(4-ethynyl-2-hydroxybenzylidene)benzohydrazide (SB-AF-08-27); Yellow solid, (120 mg, 63% yield); Rf=0.32 (Hexanes:Ethyl Acetate, 2:1); mp=185° C.; ¹H NMR (500 MHz, Acetone-d₆), δ 3.75 (s, 1H, 40%), δ 3.79 (s, 1H, 60%) 1H 6.80 (s, 1H, 40%), 6.99 (d, 1H, 40%, J=8 Hz), 7.04 (d, 1H, J=9 Hz), 7.35 (d, 1H, 32%, J=7.9 Hz), 7.41 (d, 1H, 60%, J=7.8 Hz), 7.46 (d, 1H, 36%, J=8.2 Hz), 7.59 (d, 1H, 67%, J=8.2 Hz), 7.72 (dd, 1H, 60%, J=8.2 Hz, J=1.6 Hz), 7.75 (dd, 1H, 40%, J=9 Hz, J=1.7 Hz), 8.34 (s, 1H, 37%), 8.53 (s, 1H, 63%), 9.7 (s, 1H, 50%), 10.0 (s, 1H, 20%), 11.27 (s, 1H, 50%), 11.53 (s, 1H, 80%). ¹³C NMR (175 MHz, Acetone-d₆) 113.0, 117.5, 119.7, 126.3, 129.5, 130.1, 132.6, 134.6, 135.2, 138.8, 147.8, 158.8, 162.4. HRMS (TOF) m/z calcd for C₁₆H₁₀Br₂N₂O₂H⁺: 420.9186, found: 420.9182 (Δ=−1.01 ppm).

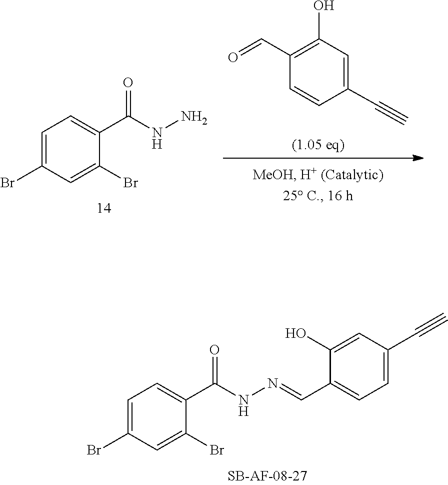 [SMILES: NNC(=O)c1ccc(Br)cc1Br ; C#Cc1ccc(C=O)c(O)c1 ; C#Cc1ccc(/C=N/NC(=O)c2ccc(Br)cc2Br)c(O)c1]

3,4-Dibromo-N′-(4-ethynyl-2-hydroxybenzylidene)benzohydrazide (SB-AF-13-27); Orange solid (98 mg, 57% yield); Rf=0.34 (Hexanes:Ethyl Acetate, 2:1); mp=204° C.; ¹H NMR (500 MHz, Acetone-d₆) δ 3.8 (s, 1H), 7.06 (d, 2H, J=10 Hz), 7.40 (d, 1H, J=10 Hz), 7.93 (d, 2H, J=2.12 Hz), 8.31 (s, 1H), 8.61 (s, 1H), 11.55 (s, 1H), 11.66 (s, 1H). ¹³C NMR (175 MHz, Acetone-d₆) δ 113.0, 117.5, 119.7, 126.3, 129.5, 130.1, 132.6, 134.6, 135.2, 138.8, 147.8, 158.8, 162.4. HRMS (TOF) m/z calcd for C₁₆H₁₀Br₂N₂O₂H⁺: 420.9182, found: 420.9147 (Δ=1.04 ppm).

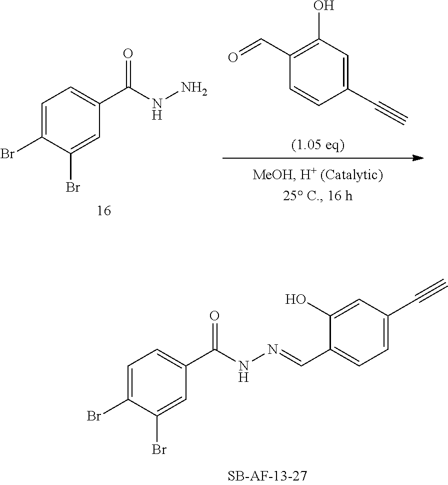 [SMILES: NNC(=O)c1ccc(Br)c(Br)c1 ; C#Cc1ccc(C=O)c(O)c1 ; C#Cc1ccc(/C=N/NC(=O)c2ccc(Br)c(Br)c2)c(O)c1]

3,5-Dibromo-N′-(4-ethynyl-2-hydroxybenzylidene)-2-hydroxybenzohydrazide (SB-AF-12-27); Yellow solid (56 mg, 84% yield), Rf=0.34 (Hexanes:Ethyl Acetate, 2:1), 2:1 mp=204° C.; ¹H NMR (500 MHz, DMSO-d₆) δ 4.3 (s, 1H), 7.01 (m, 2H), 7.61 (d, 1H, J=10 Hz), 8.01 (s, 1H), 8.18 (s, 1H), 8.72 (s, 1H), 11.04 (s, 1H), 13.03 (s, 1H). ¹³C NMR (175 MHz, DMSO-d₆) δ 82.7, 83.6, 109.9, 112.6, 114.7, 116.9, 117.2, 119.5, 129.35, 131.62, 135.11, 138.28, 147.99, 156.7, 158.0, 164.3; HRMS (TOF) m/z calcd for C₁₆H₁₀Br₂N₂O₃H⁺: 436.9131, found: 436.9127 (Δ=0.79 ppm).

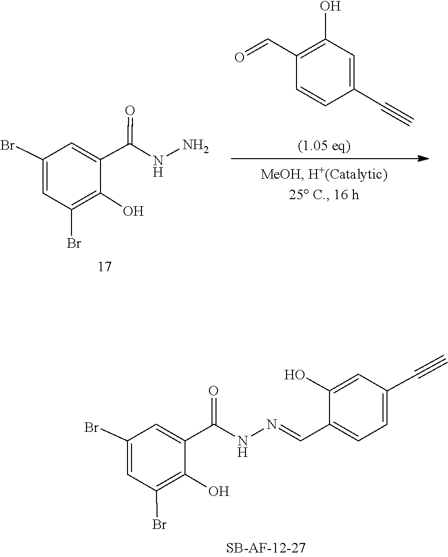 [SMILES: NNC(=O)c1cc(Br)cc(Br)c1O ; C#Cc1ccc(C=O)c(O)c1 ; C#Cc1ccc(/C=N/NC(=O)c2cc(Br)cc(Br)c2O)c(O)c1]

4-Ethynyl-N′-(4-ethynyl-2-hydroxybenzylidene)benzohydrazide (SB-AF-36-27); Brown solid (98 mg, 57% yield), mp=204° C.; Rf=0.34 (Hexanes:Ethyl Acetate, 2:1); ¹H NMR (500 MHz, DMSO-d₆) δ 4.3 (s, 1H), 4.4 (s, 1H), 7.01 (m, 2H), 7.64 (m, 3H), 7.93 (d, 2H, J=2.12 Hz), 8.66 (s, 1H), 11.27 (s, 1H), 12.21 (s, 1H). ¹³C NMR (175 MHz, DMSO-d₆) δ 82.7, 83.2, 83.6, 83.8, 119.6, 120.4, 123.3, 124.6, 125.6, 128.4, 129.4, 132.3, 133.2, 147.2, 157.4, 162.5. HRMS (TOF) m/z calcd for C₁₈H₁₂N₂O₂H⁺: 289.0972, found: 298.0982 (Δ=−3.67 ppm).

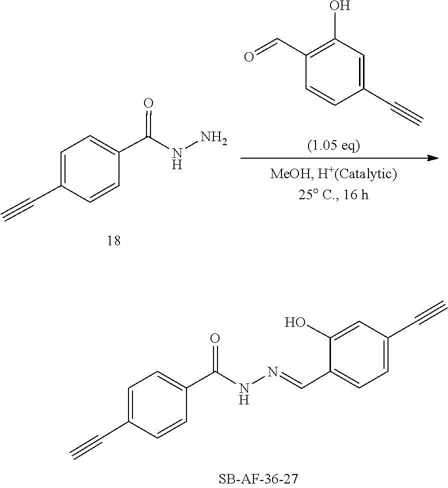 [SMILES: C#Cc1ccc(C(=O)NN)cc1 ; C#Cc1ccc(C=O)c(O)c1 ; C#Cc1ccc(C(=O)N/N=C/c2ccc(C#C)cc2O)cc1]

4-Bromo-N′-(4-ethynyl-2-hydroxybenzylidene)-3-hydroxybenzohydrazide (SB-AF-46-27); Yellow solid, (86% yield); Rf=0.35 (Hexanes:Ethyl Acetate, 2:1); mp 230° C.; ¹H NMR (700 MHz, DMSO-d₆) δ 4.31 (s, 1H), 7.06-6.97 (m, 2H), 7.31 (d, J=7.0 Hz, 1H), 7.49 (s, 1H), 7.60 (d, J=7.9 Hz, 1H), 7.66 (d, J=8.2 Hz, 1H), 8.64 (s, 1H), 10.67 (s, 1H), 11.29 (s, 1H), 12.14 (s, 1H). ¹³C NMR (176 MHz, DMSO-d₆) δ 82.78, 83.63, 114.08, 116.11, 119.62, 120.39, 123.29, 124.60, 129.53, 133.45, 133.82, 147.21, 154.76, 157.44, 162.64; HRMS (TOF) calcd for C₁₆H₁₁BrN₂O₂H⁺: 343.0076, found 343.0067 (Δ=−0.9 ppm).

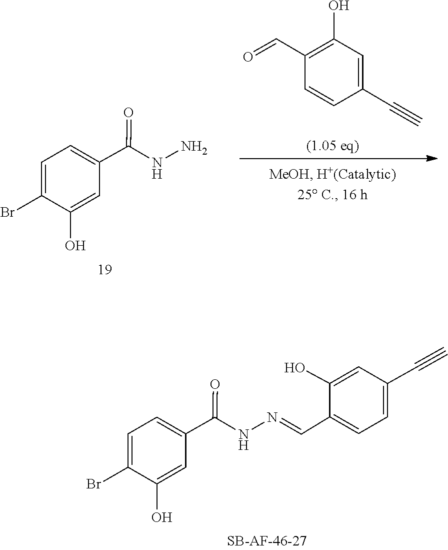 [SMILES: NNC(=O)c1ccc(Br)c(O)c1 ; C#Cc1ccc(C=O)c(O)c1 ; C#Cc1ccc(/C=N/NC(=O)c2ccc(Br)c(O)c2)c(O)c1]

4-Bromo-N′-(3-ethynyl-2-hydroxybenzylidene)benzohydrazide (SB-AF-13-28); Off-White solid, (98% yield); Rf=0.35 (Hexanes:Ethyl Acetate, 2:1); mp 185-186° C.; ¹H NMR (500 MHz, DMSO-d₆) δ 4.32 (s, 1H), 6.96 (t, J=7.7 Hz, 1H) 12.29 (s, 1H), 7.48 (d, J=7.4 Hz, 1H), 7.55 (d, J=6.8 Hz, 1H), 7.80 (d, J=8.4 Hz, 2H), 8.60 (s, 1H), 12.29 (s, 1H), 12.40 (s, 1H). ¹³C NMR (176 MHz, DMSO-d₆) δ 79.95, 85.53, 110.85, 118.76, 119.83, 126.52, 130.27, 131.97, 132.05, 132.17, 135.18, 135.46, 149.75, 159.54, 162.44; HRMS (TOF) calcd for C₁₆H₁₁BrN₂O₂H⁺: 344.0116, found 344.0108 (Δ=−2.41 ppm).

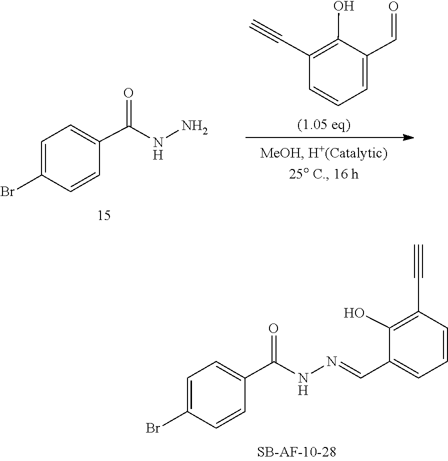 [SMILES: NNC(=O)c1ccc(Br)cc1 ; C#Cc1cccc(C=O)c1O ; C#Cc1cccc(/C=N/NC(=O)c2ccc(Br)cc2)c1O]

3,4-Dibromo-N′-(3-ethynyl-2-hydroxybenzylidene)benzohydrazide (SB-AF-13-28); Light-yellow solid, (55% yield); Rf=0.38 (Hexanes:Ethyl Acetate, 2:1); mp 195-196° C.; ¹H NMR (400 MHz, DMSO-d₆) δ 4.33 (s, 1H), 6.97 (t, J=7.7 Hz, 1H), 7.48 (dd, J=7.6, 1.5 Hz, 1H), 7.57 (dd, J=7.7, 1.5 Hz, 1H), 7.88 (dd, J=8.3, 2.0 Hz, 1H), 7.99 (d, J=8.3 Hz, 1H), 8.31 (d, J=2.0 Hz, 1H), 8.59 (s, 1H), 12.18 (s, 1H), 12.45 (s, 1H); ), ¹³C NMR (176 MHz, DMSO-d₆) δ 80.82, 87.13, 118.59, 119.68, 124.08, 124.12, 124.77, 128.81, 129.03, 132.09, 132.05, 133.62, 134.56, 148.90, 159.12, 162.10; HRMS (TOF) calcd for C₁₆H₁₀Br2N₂O₂H⁺: 421.9216, found 421.9213 (Δ=−0.73 ppm).

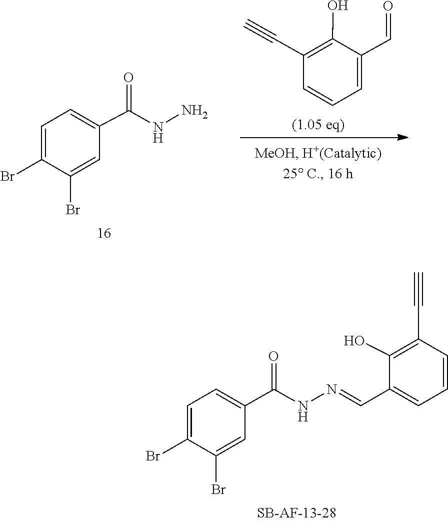 [SMILES: NNC(=O)c1ccc(Br)c(Br)c1 ; C#Cc1cccc(C=O)c1O ; C#Cc1cccc(/C=N/NC(=O)c2ccc(Br)c(Br)c2)c1O]

2,4-Dibromo-N′-(2-ethynyl-6-hydroxybenzylidene)benzohydrazide (SB-AF-08-29); White powder, (80% yield); Rf=0.35 (Hexanes:Ethyl Acetate, 2:1); mp Above 230° C.; ¹H NMR (700 MHz, Acetone-d₆) δ 4.09 (s, 1H, 70%), 4.12 (s, 1H, 30%), 6.85 (d, J=8.4 Hz, 1H, 30%), 7.04 (d, J=8.4 Hz, 1H, 70%), 7.09 (d, J=7.5 Hz, 1H, 30%), 7.14 (d, J=7.4 Hz, 1H, 70%), 7.29 (t, J=8.0 Hz, 1H, 30%), 7.37 (t, J=8.0 Hz, 1H, 70%), 7.50 (d, J=8.1 Hz, 1H, 30%), 7.65 (d, J=8.1 Hz, 1H, 70%), 7.75 (dd, J=8.2, 1.8 Hz, 1H, 70%), 7.78 (dd, J=8.1, 1.8 Hz, 1H, 30%), 7.96 (d, J=1.8 Hz, 1H, 70%), 7.99 (d, J=1.7 Hz, 1H, 30%), 8.89 (s, 1H, 30%), 9.06 (s, 1H), 10.11 (s, 1H, 30%), 11.41 (s, 1H, 40%), 11.98 (s, 1H, 60%). ¹³C NMR (176 MHz, Acetone-d₆) δ 80.01, 84.29, 84.46, 117.81, 118.15, 118.19, 118.23, 119.50, 120.74, 123.12, 124.23, 124.29, 124.31, 124.36, 124.39, 129.93, 130.88, 130.93, 131.20, 131.31, 131.58, 134.95, 135.42, 135.77, 145.79, 148.51, 157.92, 159.05, 167.57; HRMS (TOF) calcd for C₁₆H₁₁Br₂N₂O₂H⁺: 421.9226, found 421.9103 (Δ=−0.02 ppm)

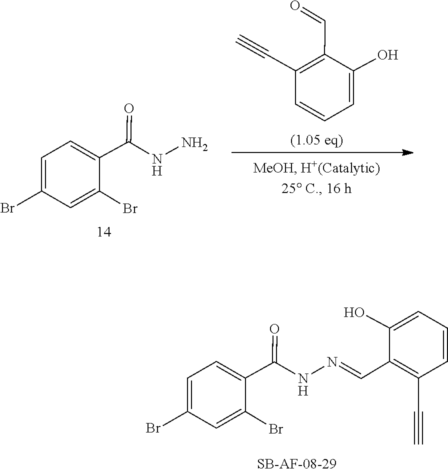 [SMILES: NNC(=O)c1ccc(Br)cc1Br ; C#Cc1cccc(O)c1C=O ; C#Cc1cccc(O)c1/C=N/NC(=O)c1ccc(Br)cc1Br]

3,4-Dibromo-N′-(2-ethynyl-6-hydroxybenzylidene)benzohydrazide (SB-AF-13-29); Light yellow solid, (78% yield); Rf=0.35 (Hexanes:Ethyl Acetate, 2:1); mp 185-185.5° C.; ¹H NMR (500 MHz, DMSO-d₆) δ 4.73 (s, 1H), 7.02 (d, J=8.3 Hz, 1H), 7.11 (d, J=7.5 Hz, 1H), 7.34 (t, J=7.9 Hz, 1H), 7.88 (dd, J=8.3, 1.8 Hz, 1H), 7.98 (d, J=8.3 Hz, 1H), 8.32 (d, J=1.8 Hz, 1H), 9.07 (s, 1H), 12.25 (s, 1H), 12.57 (s, 1H). ¹³C NMR (176 MHz, DMSO-d₆) δ 80.79, 87.11, 118.49, 118.68, 123.08, 124.62, 124.77, 128.81, 129.03, 132.06, 132.95, 133.62, 134.65, 148.89, 158.72, 161.10; HRMS (TOF) calcd for C₁₆H₁₁Br2N₂O₂H⁺: 421.9226, found 421.9213 (Δ=−3.07 ppm).

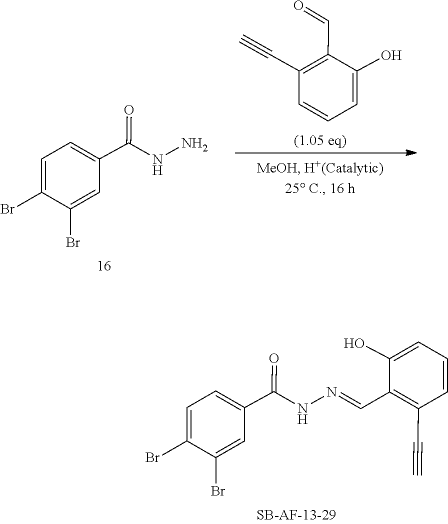 [SMILES: NNC(=O)c1ccc(Br)c(Br)c1 ; C#Cc1cccc(O)c1C=O ; C#Cc1cccc(O)c1/C=N/NC(=O)c1ccc(Br)c(Br)c1]

4-Bromo-N-(2-ethynyl-6-hydroxybenzylidene)benzohydrazide(SB-AF-10-29); Off-White solid, (61% yield); Rf=0.32 (Hexanes:Ethyl Acetate, 2:1); mp Above 230° C.; ¹H NMR (500 MHz, DMSO-d₆) δ 4.72 (s, 1H), 7.02 (d, J=8.3 Hz, 1H), 7.10 (d, J=7.5 Hz, 1H), 7.33 (t, J=8.0 Hz, 1H), 7.79 (d, J=8.5 Hz, 2H), 7.92 (d, J=8.5 Hz, 2H), 9.08 (s, 1H), 12.30 (s, 1H), 12.52 (s, 1H). ¹³C NMR (176 MHz, DMSO-d₆) δ 80.82, 87.06, 118.48, 118.78, 123.01, 124.58, 126.60, 130.27, 131.86, 131.95, 132.17, 148.54, 158.70, 162.37; HRMS (TOF) calcd for C₁₆H₁₁BrN₂O₂H⁺: 343.0076, found 343.0077 (Δ=0.1 ppm).

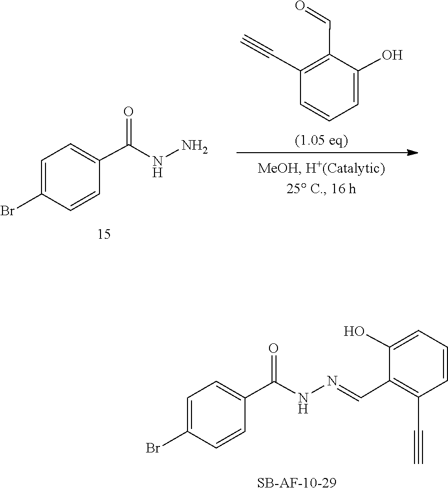 [SMILES: NNC(=O)c1ccc(Br)cc1 ; C#Cc1cccc(O)c1C=O ; C#Cc1cccc(O)c1/C=N/NC(=O)c1ccc(Br)cc1]

N′-(4-Ethynyl-2-hydroxybenzylidene)-4-(isopropoxymethyl)benzohydrazide (SB-AF-41-27); Off-white powder, (71% yield); Rf=0.38 (Hexanes:Ethyl Acetate, 2:1); mp 170-171° C.; ¹H NMR (700 MHz, DMSO-d₆) δ 1.17 (d, J=5.1 Hz, 6H), 3.81-3.55 (m, 1H), 4.32 (s, 1H), 4.55 (s, 2H), 7.09-6.93 (m, 2H), 7.48 (d, J=7.2 Hz, 2H), 7.61 (d, J=7.5 Hz, 1H), 7.93 (d, J=7.2 Hz, 2H), 8.66 (s, 1H), 11.36 (s, 1H), 12.14 (s, 1H). ¹³C NMR (176 MHz, DMSO-d₆) δ 22.51 (s) 68.96 (s), 71.12 (s), 82.75 (s), 83.65 (s), 119.62 (s), 120.41 (s), 123.28 (s), 124.52 (s), 127.52 (s), 128.12 (s), 129.60 (s), 131.95 (s), 144.09 (s), 147.01 (s), 157.45 (s), 163.13 (s). HRMS (TOF) m/z calcd for C₂₀H₂₀N₂O₃H⁺: 336.1474, found: 336.1469 (Δ=1.42 ppm).

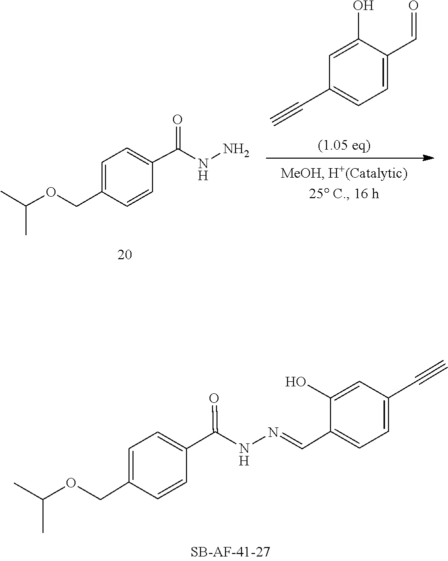 [SMILES: CC(C)OCc1ccc(C(=O)NN)cc1 ; C#Cc1ccc(C=O)c(O)c1 ; C#Cc1ccc(/C=N/NC(=O)c2ccc(COC(C)C)cc2)c(O)c1]

N′-(4-Ethynyl-2-hydroxybenzylidene)-4-(propoxymethyl)benzohydrazide (SB-AF-44-27); Off-White solid, (76% yield); Rf=0.42 (Hexanes:Ethyl Acetate, 2:1); mp 177-178° C.; ¹H NMR (500 MHz, DMSO-d₆) δ 0.91 (t, J=7.4 Hz, 3H), 1.65-1.52 (m, 2H), 3.33 (s, 1H), 3.43 (t, J=6.6 Hz, 2H), 4.32 (s, 1H), 4.55 (s, 2H), 7.12-6.93 (m, 2H), 7.49 (d, J=8.0 Hz, 2H), 7.61 (d, J=7.9 Hz, 1H), 7.94 (d, J=8.1 Hz, 2H), 8.66 (s, 1H), 11.35 (s, 1H), 12.14 (s, 1H). ¹³C NMR (176 MHz, DMSO-d₆) δ 11.07 (s), 22.93 (s), 71.61 (s), 71.97 (s), 82.75 (s), 83.65 (s), 119.62 (s), 120.40 (s), 123.28 (s), 124.53 (s), 127.61 (s), 128.17 (s), 129.59 (s), 132.11 (s), 143.55 (s), 147.01 (s), 157.45 (s), 163.12 (s); HRMS (TOF) m/z calcd for C₂₀H₂₀N₂O₃H⁺: 336.1474, found: 336.1474 (Δ=−1.67 ppm).

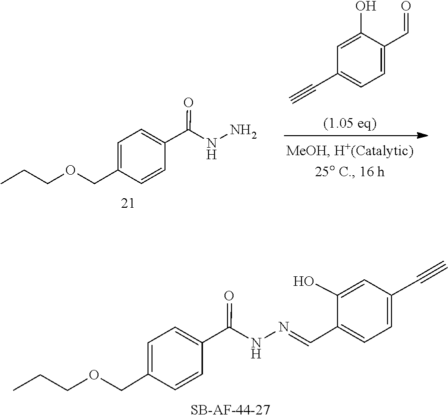 [SMILES: C#Cc1ccc(C=O)c(O)c1 ; CCCOCc1ccc(C(=O)NN)cc1 ; C#Cc1ccc(/C=N/NC(=O)c2ccc(COCCC)cc2)c(O)c1]

4-(Ethoxymethyl)-N′-(4-ethynyl-2-hydroxybenzylidene)benzohydrazide (SB-AF-40-27); Off-White powder, (89% yield); Rf=0.35 (Hexanes:Ethyl Acetate, 2:1); mp 180-181° C.; ¹H NMR (500 MHz, DMSO-d₆) δ 1.19 (t, J=7.0 Hz, 3H), 3.53 (q, J=7.0 Hz, 2H), 4.32 (s, 1H), 4.55 (s, 2H), 7.12-6.95 (m, 2H), 7.49 (d, J=8.0 Hz, 2H), 7.61 (d, J=7.9 Hz, 1H), 7.94 (d, J=8.0 Hz, 2H), 8.66 (s, 1H), 11.35 (s, 1H), 12.14 (s, 1H). ¹³C NMR (176 MHz, DMSO-d₆) δ 15.57 (s), 65.71 (s), 71.45 (s), 82.75 (s), 83.65 (s), 119.62 (s), 120.41 (s), 123.28 (s), 124.53 (s), 127.63 (s), 128.17 (s), 129.59 (s), 132.09 (s), 143.50 (s), 147.01 (s), 157.45 (s), 163.11 (s); HRMS (TOF) m/z calcd for C₂₀H₂₀N₂O₃H⁺: 332.1317, found: 332.1378 (Δ=3.67 ppm).

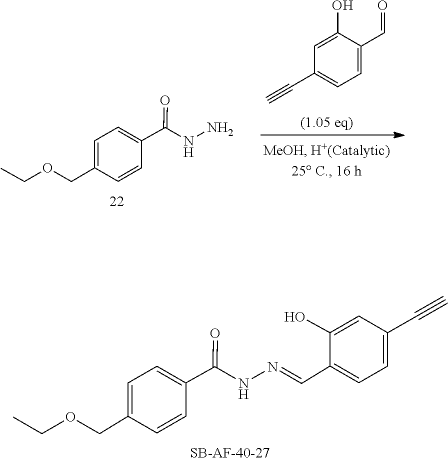 [SMILES: CCOCc1ccc(C(=O)NN)cc1 ; C#Cc1ccc(C=O)c(O)c1 ; C#Cc1ccc(/C=N/NC(=O)c2ccc(COCC)cc2)c(O)c1]

2-Hydroxy-3,5-dibromo-N-(5-ethynyl-2-hydroxybenzylidene)benzohydrazide (SB-AF-12-23); Recrystallization from acetone: hexanes (Beige solid, 70 mg, 830% yield); Rf=0.37 (Hexanes:Ethyl Acetate, 2:1); mp decomposed at 185° C.; ¹H NMR (700 MHz DMSO-d₆) δ 4.05 (s, 1H), 6.95 (d, J=8.5 Hz, 1H), 7.41 (dd, J=1.8 Hz, 8.5 Hz, 1H), 7.79 (d, J=1.8 Hz, 1H), 8.02 (d, J=2.0 Hz, 1H), 8.19 (d, J=2.0 Hz, 1H), 8.68 (s, 1H), 11.24 (s, 1H), 12.45 (br s, 1H), 12.99 (br s, 1H); ¹³C NMR (175 MHz, DMSO-d₆) δ 79.2, 83.0, 109.9, 112.4, 112.7, 116.7, 117.0, 119.3, 129.3, 131.6, 135.1, 138.8, 147.9, 156.7, 157.8, 164.3; HRMS (TOF) m/z calcd for C₁₆H₁₀Br₂N₂O₃H⁺: 436.9131, found: 436.9135 (Δ=−0.89 ppm).

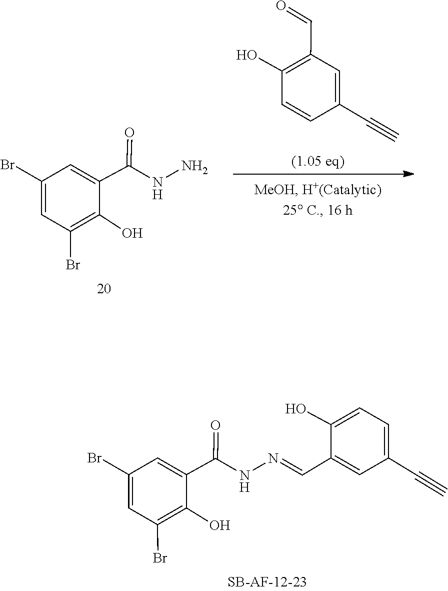 [SMILES: NNC(=O)c1cc(Br)cc(Br)c1O ; C#Cc1ccc(O)c(C=O)c1 ; C#Cc1ccc(O)c(/C=N/NC(=O)c2cc(Br)cc(Br)c2O)c1]

4-Methoxymethyl-N′-(5-ethynyl-2-hydroxybenzylidene)benzohydrazide (SB-AF-39-23); White solid (91% yield); Rf=0.37 (Hexanes:Ethyl Acetate, 2:1); mp 159-160° C.; ¹H NMR (700 MHz DMSO-d₆) δ 3.31 (s, 3H), 4.04 (s, 1H), 4.48 (s, 2H), 6.93 (d, J=8.4 Hz, 1H), 7.39 (d, J=8.4 Hz, 1H), 7.46 (d, J=7.7 Hz, 2H), 7.73 (s, 1H), 7.93 (d, J=7.7 Hz, 2H), 8.61 (s, 1H), 11.60 (s, 1H), 12.18 (s, 1H); ¹³C NMR (175 MHz, DMSO-d₆) δ 57.8, 73.0, 79.2, 83.1, 112.6, 117.0, 119.3, 127.2, 127.7, 131.7, 132.5, 134.5, 142.6, 146.6, 157.8, 162.7; HRMS (TOF) m/z calcd for C₁₈H₁₆N₂O₃H⁺: 309.1234, found: 309.1223 (Δ=−3.51 ppm).

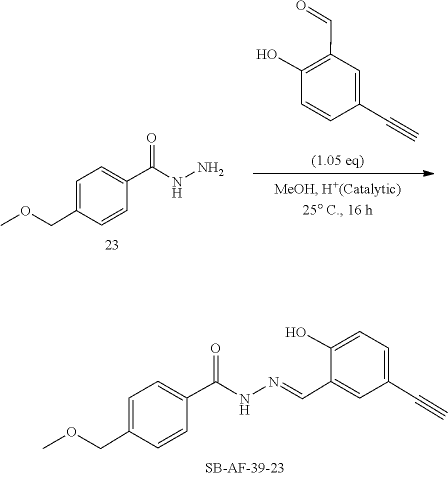 [SMILES: COCc1ccc(C(=O)NN)cc1 ; C#Cc1ccc(O)c(C=O)c1 ; C#Cc1ccc(O)c(/C=N/NC(=O)c2ccc(COC)cc2)c1]

2-Fluoro-4-(trifluoromethoxy)-N-(5-ethynyl-2-hydroxybenzylidene)benzohydrazide (SBAF-25-23); Beige solid (0.13 g, 78% yield); Rf=0.34 (Hexanes:Ethyl Acetate, 2:1); mp 159-161° C.; ¹H NMR (700 MHz, DMSO-d₆) δ 3.94 (s, 1H, 26%), 4.04 (s, 1H, 74%), 6.83 (d, J=8.5 Hz, 1H, 25%), 6.93 (d, J=8.5 Hz, 1H, 75%), 7.29 (dd, J=8.4 Hz, 2.1 Hz, 1H, 25%), 7.34 (d, J=2.1 Hz, 1H, 25%), 7.36 (d, J=8.5 Hz, 1H, 25%), 7.38-7.40 (m, 2H, 75%, 75%), 7.54 (d, J=8.9 Hz, 1H, 25%), 7.58 (d, J=8.9 Hz, 1H, 75%), 7.67 (t, J=8.1 Hz, 1H, 25%), 7.76 (d, J=2.1 Hz, 1H, 75%), 7.85 (t, J=8.1 Hz, 1H, 75%), 8.30 (s, 1H, 25%), 8.51 (s, 1H, 75%), 10.43 (s, 1H, 22%), 11.30 (s, 1H, 78%), 12.14 (s, 1H, 26%), 12.21 (s, 1H, 74%); ¹³C NMR (175 MHz, DMSO-d₆) δ 79.1, 79.4, 83.2, 109.4, 109.6, 110.1, 110.3, 112.80, 112.85, 116.9, 117.20, 117.23, 117.4, 117.8, 119.30, 119.34, 119.5, 120.2, 120.75, 120.80, 122.20, 122.22, 122.3, 123.5, 123.6, 130.1, 131.20, 131.22, 132.15, 132.17, 132.3, 134.6, 135.0, 141.5, 146.9, 149.90, 149.94, 150.55, 150.61, 157.1, 157.9, 159.0, 159.3, 159.5, 160.4, 165.9; ¹⁹F NMR (376 MHz DMSO-d₆) δ−56.95 (s, 3F), −57.03 (s, 3F), −109.20 (s, 1F), −109.45 (s, 1F); HRMS (TOF) m/z calcd for C₁₇H₁₀F₄N₂O₃H⁺:367.0700, found: 367.0704 (Δ=−0.99 ppm).

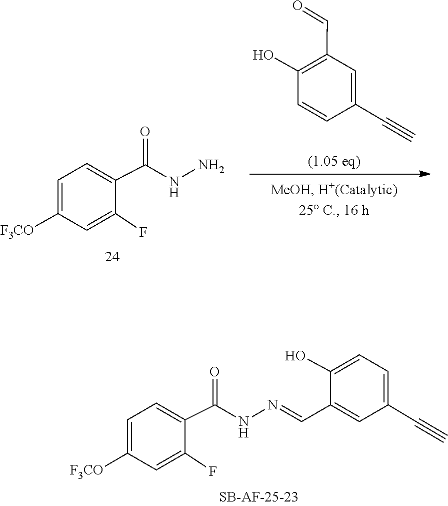 [SMILES: NNC(=O)c1ccc(OC(F)(F)F)cc1F ; C#Cc1ccc(O)c(C=O)c1 ; C#Cc1ccc(O)c(/C=N/NC(=O)c2ccc(OC(F)(F)F)cc2F)c1]

4-(3-(Trifluoromethyl)-3H-diazirin-3-yl)-N′-(5-ethynyl-2-hydroxybenzylidene) benzohydrazide (SB-AF-43-23); Beige solid (19 mg, 63% yield) Rf=0.38 (Hexanes:Ethyl Acetate, 2:1); mp decomposed at 131° C.; ¹H NMR (500 MHz DMSO-d₆) δ 4.03 (s, 1H), 6.93 (d, 1H, J=8.5 Hz), 7.39 (dd, 1H, J=8.5, 2.0 Hz), 7.45 (d, 2H, J=8.2 Hz), 7.75 (d, 1H, 2.0 Hz), 8.04 (d, 2H, J=8.5 .Hz), 8.62 (s, 1H), 11.47 (s, 1H), 12.28 (s, 1H); ¹³C NMR (125 MHz DMSO-d₆) δ 79.2, 83.1, 112.6, 117.0, 119.3, 120.6, 122.8, 126.6, 128.6, 131.0, 132.2, 134.3, 134.6, 146.9, 157.8, 161.8; ¹⁹F NMR (376 MHz DMSO-d₆) δ−64.40 (s, 3F); HRMS (TOF) m/z calcd for C₁₈H₁₁F₃N₄O₂H⁺: 373.0907, found: 373.0940 (Δ=−8.9 ppm).

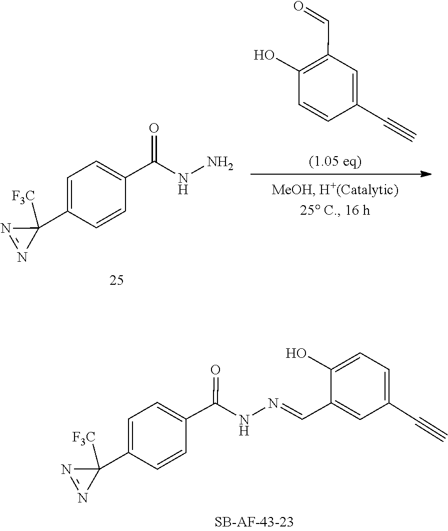 [SMILES: NNC(=O)c1ccc(C2(C(F)(F)F)N=N2)cc1 ; C#Cc1ccc(O)c(C=O)c1 ; C#Cc1ccc(O)c(/C=N/NC(=O)c2ccc(C3(C(F)(F)F)N=N3)cc2)c1]

### 2,4-Dibromo-N′-((4-hydroxy-[1,1′-biphenyl]-3-yl)methylene)benzohydrazide (SB-AF-08-24)

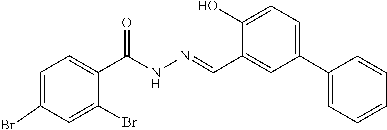 [SMILES: O=C(N/N=C/c1cc(-c2ccccc2)ccc1O)c1ccc(Br)cc1Br]

SB-AF-08-24 was synthesized by the condensation reaction of hydrazide 14 with commercially available 4-hydroxy-5-formyl-1,1′-biphenyl in the same manner as the synthesis of SB-AF-10-23.

White solid (0.07 g, 49% yield); mp 234-236° C.; ¹H NMR (400 MHz, DMSO-d₆) δ 6.89 (d, J=8.5 Hz, 1H, 36%), 7.03 (d, J=8.5 Hz, 1H, 64%), 7.27-7.33 (m, 1H, 100%), 7.39-7.52 (m, 3H, 100%, 100%, 100%), 7.54-7.57 (m, 2H, 100%, 32%), 7.61-7.64 (m, 2H, 100%, 82%), 7.70-7.74 (m, 1H, 100%), 7.90 (d, J=2.2 Hz, 1H, 68%), 8.01 (dd, J=6.6 Hz, 1.6 Hz, 1H, 100%), 8.23 (s, 1H, 36%), 8.55 (s, 1H, 64%), 9.99 (s, 1H, 37%), 11.08 (s, 1H, 63%), 12.20 (s, 1H, 100%); ¹³CNMR (126 MHz, DMSO-d₆) δ168.3, 162.4, 157.0, 156.1, 148.0, 143.0, 139.4, 139.2, 137.4, 136.1, 134.8, 134.1, 131.5, 131.1, 130.9, 130.8, 130.7, 130.1, 130.0, 129.3, 128.9, 126.9, 126.8, 126.1, 125.8, 125.7, 123.8, 122.6, 120.7, 1120.0, 119.5, 119.0, 117.0, 116.8; HRMS (TOF) m/z calcd for C₂₀H₁₄Br₂N₂O₂H⁺: 472.9495, found: 472.9508 (Δ=−2.75 ppm).

### 3,4-dibromo-N′-((4-hydroxy-[1,1′-biphenyl]-3-yl)methylene)benzohydrazide (SB-AF-13-24)

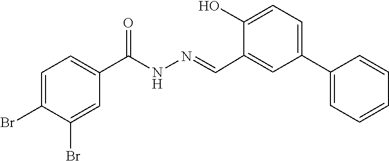 [SMILES: O=C(N/N=C/c1cc(-c2ccccc2)ccc1O)c1ccc(Br)c(Br)c1]

SB-AF-13-24 was synthesized by the condensation reaction of hydrazide 16 with commercially available 4-hydroxy-5-formyl-1,1′-biphenyl in the same manner as the synthesis of SB-AF-10-23.

Beige solid (0.10 g, 63% yield); mp>230° C.; ¹H NMR (500 MHz, DMSO-d₆) δ 7.03 (d, J=8.5 Hz, 1H), 7.31 (t, J=7.4 Hz, 1H), 7.44 (t, J=7.4 Hz, 2H), 7.60-7.63 (m, 3H), 7.85-7.87 (m, 2H), 7.95 (d, J=8.5 Hz, 1H), 8.29 (s, 1H), 8.71 (s, 1H), 11.25 (s, 1H), 12.28 (s, 1H); ¹³C NMR (126 MHz, DMSO-d₆) δ 117.0, 119.1, 124.2, 126.2, 126.9, 127.0, 128.0, 128.5, 128.9, 129.9, 131.5, 132.5, 133.7, 134.1, 139.4, 148.3, 157.1, 160.7; HRMS (TOF) m/z calcd for C₂₀H₁₄Br₂N₂O₂H⁺: 472.9495, found: 472.9498 (Δ=−0.76 ppm).

### 4-(Methoxymethyl)benzohydrazide (26)

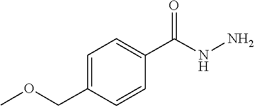

Methyl 4-(bromomethyl)benzoate (2.0 g, 8.7 mmol) was refluxed in methanol (36 mL) in the presence of sulfuric acid (2 mL) for 72 hours. The reaction mixture was cooled to room temperature, followed by the addition of hydrazine monohydrate (12 g, 435 mmol), and the reaction mixture was heated at 90° C. for 24 hours. Upon completion, the reaction mixture was cooled to room temperature and concentrated in a rotary evaporator, and 20 mL of ware was added. The product was extracted using ethyl acetate (3×30 mL). The organic layers were combined, dried using anhydrous magnesium sulfate, filtered, and concentrated in a rotary evaporator. The product was dried under vacuum to give a white solid (1.4 g, 89% yield); mp 72-74° C.; ¹H NMR (300 MHz DMSO-d₆) δ 3.43 (s, 3H), 4.43 (s, 2H), 4.47 (s, 2H), 7.36 (d, 2H, J=8.1 Hz), 7.79 (d, 2H, J=8.1 Hz), 9.75 (s, 1H); ¹³C NMR (175 MHz DMSO-d₆) δ 57.7, 73.1, 126.9, 127.1, 132.6, 141.4, 165.7; MS (ESI) m/z 181.1 (M+1).

The same procedure was used for the synthesis of 27, 28 and 29.

### 4-(ethoxymethyl)benzohydrazide (27)

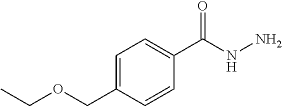

White solid (0.16 g, 83% yield); m.p. 70-71° C.; ¹H NMR (300 MHz DMSO-d₆) δ 1.14 (t, J=7.0 Hz, 3H), 3.47 (q, J=7.0 Hz, 2H), 4.47 (s, 4H), 7.36 (d, J=8.4 Hz, 2H), 7.78 (d, J=8.4 Hz, 2H), 9.74 (s, 1H); ¹³C NMR (125 MHz DMSO-d₆) δ 15.1, 66.0, 71.9, 127.0, 127.5, 142.8, 168.4; MS (ESI) m/z 195.1 (M+1).

### 4-(isopropoxymethyl)benzohydrazide (28)

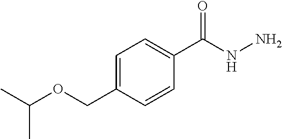

Pinkish brown solid (0.25 g, 93% yield); m.p. 40-41° C., ¹H NMR (300 MHz DMSO-d₆) δ 1.12 (d, 6H, J=6 Hz), 3.61 (sept 1H, J=6 Hz), 4.47 (s, 4H), 7.35 (d, 2H, J=8.2 Hz), 7.77 (d, 2H, J=8.2 Hz), 9.74 (s, 1H).

### 4-(propoxymethyl)benzohydrazide (29)

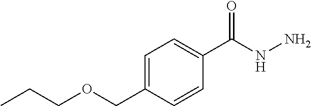

White solid (0.18 g, 86% yield); m.p. 69-71° C., ¹H NMR (300 MHz DMSO-d₆) δ 0.87 (t, J=7.4 Hz, 3H), 1.48-1.60 (m, 2H), 3.37 (t, J=6.6 Hz, 2H), 4.47 (s, 4H), 7.36 (d, J=8.0 Hz, 2H), 7.78 (d, J=8.0 Hz, 2H), 9.74 (s, 1H); MS (ESI) m/z 209.1 (M+1).

### 4-Methoxymethyl-N′-(3,5-dibromo-2-hydroxybenzylidene)benzohydrazide (SB-AF-39-17)

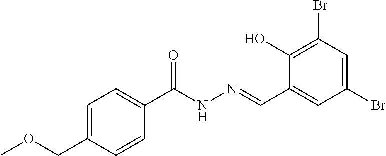 [SMILES: COCc1ccc(C(=O)N/N=C/c2cc(Br)cc(Br)c2O)cc1]

4-(Methoxymethyl)benzohydrazide (26, 1 g, 5.6 mmol) was coupled with 3,5-dibromosalicylaldehyde (1.6 g, 5.8 mmol) in methanol in the presence of the catalytic amount of acetic acid. The reaction mixture was stirred at room temperature overnight. The reaction was quenched by adding 15 mL of water. The precipitate formed was filtered, washed with water, and dried to give the crude product. The crude product was washed with DCM (30 mL) and hexanes (30 mL) to give SB-AF-39-17 as a beige solid (2.1 g, 85% yield); mp>230° C.; ¹H NMR (700 MHz DMSO-d₆) δ 4.49 (s, 2H), 7.48 (d, 2H, J=8.1 Hz), 7.81 (dd, 2H, J=9.9 Hz, 2.1 Hz), 7.94 (d, 2H, J=8.1 Hz), 8.52 (s, 1H), 12.53 (s, 1H), 12.75 (s, 1H); ¹³C NMR (175 MHz DMSO-d₆) δ 57.8, 73.0, 110.4, 111.2, 121.0, 127.3, 127.8, 131.1, 132.1, 135.5, 143.0, 147.0, 153.7, 162.8; HRMS (TOF) m/z calcd for C₁₆H₄Br₂N₂O₃H⁺:440.9444, found: 440.9448 (Δ=−0.88 ppm).

The same procedure was used for the synthesis of SB-AF-39-13, SB-AF-39-15, SB-AF-39-22, SB-AF-39-17, SB-AF-40-17, SB-AF-40-15, SB-AF-40-22, SB-AF-44-17, SB-AF-44-15, SB-AF-41-17, SB-AF-41-15, SB-AF-41-13.

### 4-Methoxymethyl-N′-(4-bromo-2-hydroxybenzylidene)benzohydrazide (SB-AF-39-13)

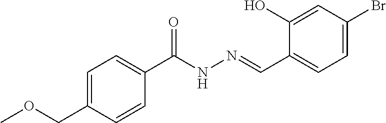 [SMILES: COCc1ccc(C(=O)N/N=C/c2ccc(Br)cc2O)cc1]

White solid (0.12 g, 86% yield); mp 198-199° C.; ¹H NMR (500 MHz DMSO-d₆) δ 3.31 (s, 3H), 4.48 (s, 2H), 7.10 (dd, 1H, J=8.3, 1.6 Hz), 7.13 (d, 1H, J=1.6 Hz), 7.46 (d, 2H, J=8.1 Hz), 7.54 (d, 1H, J=8.3 Hz), 7.91 (d, 2H, J=8.1 Hz), 8.61 (s, 1H), 11.53 (s, 1H), 12.11 (s, 1H); ¹³C NMR (125 MHz DMSO-d₆) δ 57.8, 73.0, 118.5, 119.1, 122.4, 123.9, 127.2, 127.7, 130.4, 131.7, 142.6, 146.5, 158.1, 162.6; HRMS (TOF) m/z calcd for C₁₆H₁₅BrN₂O₃H⁺: 363.0339, found: 363.0345 (Δ=−1.59 ppm).

### 4-Methoxymethyl-N′-(5-bromo-2-hydroxybenzylidene)benzohydrazide (SB-AF-39-15)

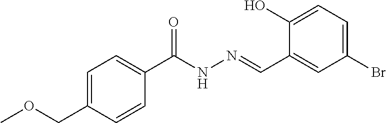 [SMILES: COCc1ccc(C(=O)N/N=C/c2cc(Br)ccc2O)cc1]

Light yellow solid (0.14 g, 70% yield); mp 188-189° C.; ¹H NMR (700 MHz, DMSO-d₆) δ 12.18 (s, 1H), 11.31 (s, 1H), 8.62 (s, 1H), 7.93 (d, J=8.0 Hz, 2H), 7.80 (d, J=2.2 Hz, 1H), 7.47 (d, J=8.0 Hz, 2H), 7.43 (dd, J=8.7, 2.3 Hz, 1H), 6.91 (d, J=8.7 Hz, 1H), 4.50 (s, 2H), 3.33 (s, 3H); ¹³C NMR (176 MHz, DMSO-d₆) δ 162.7, 156.4, 145.6, 142.6, 133.6, 131.7, 130.5, 127.7, 127.2, 121.3, 118.7, 110.5, 73.0, 57.8; HRMS (TOF) m/z calcd for C₁₆H₁₅BrN₂O₃H⁺: 363.0321, found: 363.0339 (Δ=4.88 ppm).

### 4-Methoxymethyl-N′-(3-bromo-2-hydroxybenzylidene)benzohydrazide (SB-AF-39-22)

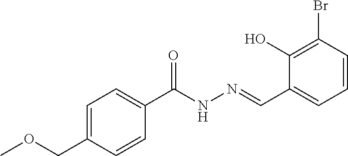 [SMILES: COCc1ccc(C(=O)N/N=C/c2cccc(Br)c2O)cc1]

White solid (94 mg, 91% yield); m.p. 187-188° C., ¹H NMR (400 MHz, DMSO-d₆) δ 3.32 (s, 3H), 4.49 (s, 2H), 6.90 (t, J=7.8 Hz, 1H), 7.47-7.51 (m, 3H), 7.62 (dd, J=7.8 Hz, 1.1 Hz, 1H), 7.94 (d, J=8.2 Hz, 2H), 8.57 (s, 1H), 12.36 (s, 1H), 12.61 (s, 1H); C NMR (100 MHz, DMSO-d₆) δ 57.7, 72.9, 109.9, 119.4, 120.5, 127.3, 127.7, 130.3, 131.2, 134.3, 142.8, 148.6, 154.2, 162.6.

### 4-Ethoxymethyl-N′-(3,5-dibromo-2-hydroxybenzylidene)benzohydrazide (SB-AF-40-17)

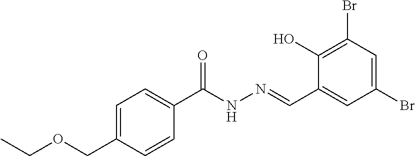 [SMILES: CCOCc1ccc(C(=O)N/N=C/c2cc(Br)cc(Br)c2O)cc1]

Light yellow solid (0.16 g, 86% yield); mp 205-207° C.; ¹H NMR (700 MHz, DMSO-d₆) δ 12.77 (s, 1H), 12.54 (s, 1H), 8.54 (s, 1H), 7.95 (d, J=8.1 Hz, 2H), 7.82 (dd, J=9.8, 2.2 Hz, 2H), 7.49 (d, J=8.1 Hz, 2H), 4.55 (s, 2H), 3.52 (q, J=7.0 Hz, 2H), 1.18 (t, J=7.0 Hz, 3H); ¹³C NMR (176 MHz, DMSO-d₆) δ 162.8, 153.7, 147.0, 143.4, 135.5, 132.1, 131.0, 127.8, 127.2, 121.0, 111.2, 110.4, 70.9, 65.3, 15.1; HRMS (TOF) m/z calcd for C₁₇H₁₆Br₂N₂O₃H⁺: 454.9611, found: 454.9600 (Δ=−2.25 ppm).

### 4-Ethoxymethyl-N′-(5-bromo-2-hydroxybenzylidene)benzohydrazide (SB-AF-40-15)

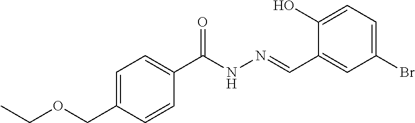 [SMILES: CCOCc1ccc(C(=O)N/N=C/c2cc(Br)ccc2O)cc1]

Beige solid (0.10 g, 67% yield); 195-196° C.; ¹H NMR (500 MHz, DMSO-d₆) δ 1.16 (t, J=7.0 Hz, 3H), 3.50 (q, J=7.0 Hz, 2H), 4.52 (s, 2H), 7.10 (dd, J=8.3, 1.5 Hz, 1H), 7.13 (d, J=1.5 Hz, 1H), 7.46 (d, J=8.1 Hz, 2H), 7.54 (d, J=8.3 Hz, 1H), 7.91 (d, J=8.1 Hz, 2H), 8.61 (s, 1H), 11.52 (br.s, 1H), 12.10 (br.s, 1H); ¹³C NMR (126 MHz, DMSO-d₆) δ 15.1, 65.2, 70.9, 118.5, 119.1, 122.4, 123.8, 127.1, 127.6, 130.4, 131.6, 143.0, 146.5, 158.0, 162.6; HRMS (TOF) m/z calcd for C₁₇H₁₇BrN₂O₃H⁺: 377.0495, found: 377.0502 (Δ=1.90 ppm).

### 4-Ethoxymethyl-N′-(3-bromo-2-hydroxybenzylidene)benzohydrazide (SB-AF-40-22)

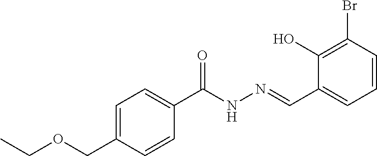 [SMILES: CCOCc1ccc(C(=O)N/N=C/c2cccc(Br)c2O)cc1]

White solid (73 mg, 75% yield); m.p. 176-177° C.; ¹H NMR (400 MHz, DMSO-d₆) δ 1.16 (t, J=7.0 Hz, 3H), 3.51 (q, J=7.0 Hz, 2H), 4.53 (s, 2H), 6.90 (t, J=7.8 Hz, 1H), 7.47-7.51 (m, 3H), 7.61 (d, J=7.8 Hz, 1H), 7.93 (d, J=8.1 Hz, 2H), 8.57 (s, 1H), 12.35 (s, 1H), 12.61 (s, 1H); ¹³C NMR (100 MHz, DMSO-d₆) δ 15.1, 65.2, 70.9, 109.9, 119.4, 120.5, 127.2, 127.7, 130.3, 131.2, 134.3, 143.3, 148.6, 154.2, 162.6.

### 4-Propoxymethyl-N′-(3,5-dibromo-2-hydroxybenzylidene)benzohydrazide (SB-AF-44-17)

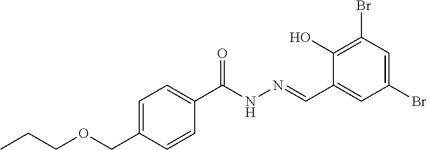 [SMILES: CCCOCc1ccc(C(=O)N/N=C/c2cc(Br)cc(Br)c2O)cc1]

White solid (0.10 g, 45% yield); mp 190-192° C.; ¹H NMR (700 MHz, DMSO-d₆) δ 12.77 (s, 1H), 12.53 (s, 1H), 8.53 (s, 1H), 7.94 (d, J=8.1 Hz, 2H), 7.82 (dd, J=8.9, 2.2 Hz, 2H), 7.49 (d, J=8.1 Hz, 2H), 4.54 (s, 2H), 3.42 (t, J=6.6 Hz, 2H), 1.78-1.42 (m, 2H), 0.90 (t, J=7.4 Hz, 3H); ¹³C NMR (176 MHz, DMSO-d₆) δ 162.8, 153.7, 147.0, 143.5, 135.5, 132.1, 131.0, 127.8, 127.2, 121.0, 111.2, 110.4, 71.5, 71.1, 22.5, 10.6; HRMS (TOF) m/z calcd for C₁₈H₁₈Br₂N₂O₃H⁺: 468.9765, found: 468.9757 (Δ=−1.63 ppm).

### 4-Propoxymethyl-N′-(5-bromo-2-hydroxybenzylidene)benzohydrazide (SB-AF-44-15)

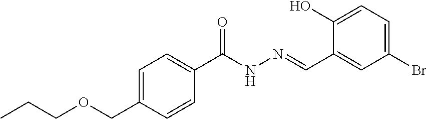 [SMILES: CCCOCc1ccc(C(=O)N/N=C/c2cc(Br)ccc2O)cc1]

White solid (0.12 g, 64% yield); mp 175-176° C.; ¹H NMR (500 MHz, DMSO-d₆) δ 0.88 (t, J=7.4 Hz, 3H), 1.52-1.59 (m, 2H), 3.40 (t, J=6.6 Hz, 2H), 4.52 (s, 2H), 6.89 (d, J=8.7 Hz, 1H), 7.42 (dd, J=8.7, 2.5 Hz, 1H), 7.46 (d, J=8.1 Hz, 2H), 7.78 (d, J=2.5 Hz, 1H), 7.92 (d, J=8.1 Hz, 2H), 8.60 (s, 1H), 11.30 (s, 1H), 12.16 (s, 1H); ¹³C NMR (126 MHz, DMSO-d₆) δ 10.6, 22.4, 71.1, 71.5, 110.4, 118.7, 121.3, 127.1, 127.7, 130.4, 131.6, 133.5, 143.1, 145.6, 156.4, 162.7; HRMS (TOF) m/z calcd for C₁₈H₁₉BrN₂O₃H⁺: 391.0648, found: 391.0652 (Δ=0.87 ppm).

### 4-Isopropoxymethyl-N′-(3,5-dibromo-2-hydroxybenzylidene)benzohydrazide (SB-AF-41-17)

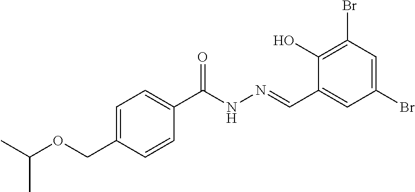 [SMILES: CC(C)OCc1ccc(C(=O)N/N=C/c2cc(Br)cc(Br)c2O)cc1]

Yellow solid (0.12 g, 70% yield); mp 186-187° C.; 1H NMR (700 MHz, DMSO-d₆) δ 12.77 (s, 1H), 12.52 (s, 1H), 8.54 (s, 1H), 7.94 (d, J=8.2 Hz, 2H), 7.82 (dd, J=8.0, 2.2 Hz, 2H), 7.49 (d, J=8.2 Hz, 2H), 4.55 (s, 2H), 3.67 (hept, J=6.1 Hz, 1H), 1.17 (d, J=6.1 Hz, 6H); ¹³C NMR (175 MHz, DMSO-d₆) δ 162.8, 153.7, 146.9, 144.0, 135.5, 132.1, 130.8, 127.8, 127.1, 121.0, 111.2, 110.4, 70.7, 68.5, 22.0; HRMS (TOF) m/z calcd for C₁₈H₁₈Br₂N₂O₃H⁺: 468.9757, found: 468.9771 (Δ=−2.98 ppm).

### 4-Isopropoxymethyl-N′-(5-bromo-2-hydroxybenzylidene)benzohydrazide (SB-AF-41-15)

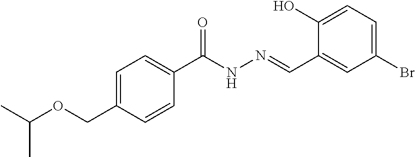 [SMILES: CC(C)OCc1ccc(C(=O)N/N=C/c2cc(Br)ccc2O)cc1]

Yellow solid (0.11 g, 73% yield); mp 179-181° C.; ¹H NMR (700 MHz, DMSO-d₆) δ 1.16 (d, J=6.1 Hz, 6H), 3.64 (hept, J=6.1 Hz, 1H), 4.53 (s, 2H), 6.90 (d, J=8.7 Hz, 1H), 7.42 (dd, J=8.7, 2.4 Hz, 1H), 7.47 (d, J=8.1 Hz, 2H), 7.78 (d, J=2.4 Hz, 1H), 7.91 (d, J=8.1 Hz, 2H), 8.60 (s, 1H), 11.30 (s, 1H), 12.16 (s, 1H); ¹³C NMR (176 MHz, DMSO-d₆) δ 22.0, 68.5, 70.6, 110.4, 118.7, 121.3, 127.0, 127.7, 130.5, 131.5, 133.5, 143.6, 145.6, 156.4, 162.7; HRMS (TOF) m/z calcd for C₁₈H₁₉BrN₂O₃H⁺: 391.0652, found: 391.0659 (Δ=−1.93 ppm).

### 4-Isopropoxymethyl-N′-(4-bromo-2-hydroxybenzylidene)benzohydrazide (SB-AF-41-13)

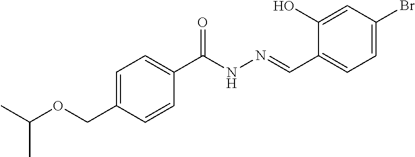 [SMILES: CC(C)OCc1ccc(C(=O)N/N=C/c2ccc(Br)cc2O)cc1]

Beige solid (0.12 g 80% yield); mp 193-195° C.; ¹H NMR (700 MHz, DMSO-d₆) δ 1.16 (d, J=6.1 Hz, 6H), 3.65 (hept, J=6.1 Hz, 1H), 4.53 (s, 2H), 7.10 (d, J=8.4 Hz, 1H), 7.13 (s, 1H), 7.46 (d, J=7.8 Hz, 2H), 7.54 (d, J=8.2 Hz, 1H), 7.91 (d, J=7.8 Hz, 2H), 8.61 (s, 1H), 11.53 (s, 1H), 12.10 (s, 1H); ¹³C NMR (176 MHz, DMSO-d₆) δ 22.0, 68.5, 70.6, 118.6, 119.1, 122.42, 123.9, 127.0, 127.6, 130.4, 131.5, 143.6, 146.5, 158.1, 162.7; HRMS (TOF) m/z calcd for C₁₈H₁₉BrN₂O₃H⁺: 391.0652, found: 391.0659 (Δ=−1.73 ppm).

### Example 5: In Vitro Activities (MIC₈₀ and K₁₀₀) of Acylhydrazones

### In Vitro Susceptibility (MIC₈₀) Assay

MICs was determined following the methods of the Clinical and Laboratory Standards Institutes (CLSI) with modifications. Yeast Nitrogen Base (YNB) medium (pH 7.0, 0.2% glucose) buffered with HEPES was used for MIC studies. HEPES was used instead of morpholinepropanesulfonic acid (MOPS), because MOPS was found to inhibit the activity of this kind of compounds. The compound was serially diluted from 16 to 0.03 μg/ml, in a 96-well plate. The inoculum was prepared as described in the CLSI protocol M27A3 guidelines. The plates were incubated at 37° C. with 5% CO₂ for 24 to 72 h and the optical density was measure at 450 nm. The MICs was determined as the lowest concentration of the compound that inhibited 80% of growth compared to the control.

### In Vitro Killing Activity (K₁₀₀) Assay

C. neoformans cells from a culture grown overnight were washed in PBS and resuspended in YNB buffered with HEPES at pH 7.4. The cells were counted, and 2×10⁴ cells were incubated with different concentration of compounds in a final volume of 10 ml with a final concentration of 0.5% DMSO. The tubes were then incubated at 37° C. with 5% CO₂ on a rotary shaker at 200 rpm. Aliquots were taken at time points and diluted, and 100-1 portions were plated onto Yeast Extract-Peptone-Dextrose (YPD) plates. YPD plates were incubated in a 30° C. incubator and after 48 h, the numbers of CFU were counted and recorded.

### DISCUSSION

The compounds described herein have potent killing activity with low or no toxicity that can be used alone or in combination of current antifungal agents to treat superficial or invasive fungal infections.

The fungal sphingolipid glucosylceramide (GlcCer) synthesis has emerged as a highly promising new target for the development of next-generation antifungal agents. GlcCer is essential for the cell division of pathogenic fungi such as C. neoformans, Candida albicans (C. albicans), and Aspergillus fumigatus (A. funigatus) and responsible for their virulence. It has been shown that fungal cells lacking GlcCer cannot replicate in neutral or alkaline environments. This finding clearly indicates the importance of GlcCer for virulence in alveolar spaces, cerebrospinal fluid, or bloodstream of the host wherein the pH is neutral or alkaline and thus makes GlcCer a promising target for drug discovery.

There is a major clinical need for new drugs due to a dramatic increase of morbidity and mortality by invasive fungal infections. Without being limited by a particular theory, the compounds contained herein decrease the synthesis of fungal but not mammalian GlcCer. This action seems to be specific to the transport of fungal ceramide species. The compounds are active in vitro against fungi, especially C. neoformans, P. murina, P. jiroveci, R. oryzae, Sporothrix schenckii, Sporothrix brasiliensis and dimorphic fungi. The compounds appear to be effective in vivo against cryptococcosis, candidiasis, sporotrichosis, and also against pneumocystosis. The compounds do not induce resistance in vitro and they are synergistic with existing antifungals.

C. albicans is resistant in vitro but not in vivo. Studies performed in this fungus have suggested that GlcCer is important for virulence but through a mechanism other than facilitating growth at neutral/alkaline pH, which is the pH used to screen our ChemBridge library. Hence, inhibition of GlcCer in C. albicans does not block fungal growth in vitro. However, because the compound still decreases GlcCer synthesis, which is required for Candida virulence, the treatment is effective in partially protecting mice from invasive candidiasis. These findings support previous studies suggesting that the effect of GlcCer in vivo during Candida infection goes beyond the regulation of fungal alkaline tolerance.

The compounds disclosed herein inhibit GlcCer synthesis; however, this lipid is most likely not the only target of these compounds. In fact, the blockage of fungal growth in alkaline pH due to the loss of GlcCer (Δgcs1 mutant) can be restored if Δgcs1 cells are shifted to an acidic environment (Singh A. et al. 2012). This can occur even after the cells are left in cell cycle arrest for 72 hours. This means that the lack of GlcCer has a “static” effect on cell growth. However, the compounds disclosed herein kill fungal cells. One explanation for this effect is that treatment with the compound acutely leads to the accumulation of sphingosines, which is highly toxic to fungal cells (Chung, N. et al. 2001; Chung, N. et al. 2000). The accumulation of sphingosine species is not present when Gcs1 is deleted (Rittershaus, P. C. 2006) or in mammalian cells treated with compound. Thus, the effect seems to go beyond the inhibition of GlcCer and this may account for the fungal killing effect exerted by the compounds and not by the absence of GlcCer.

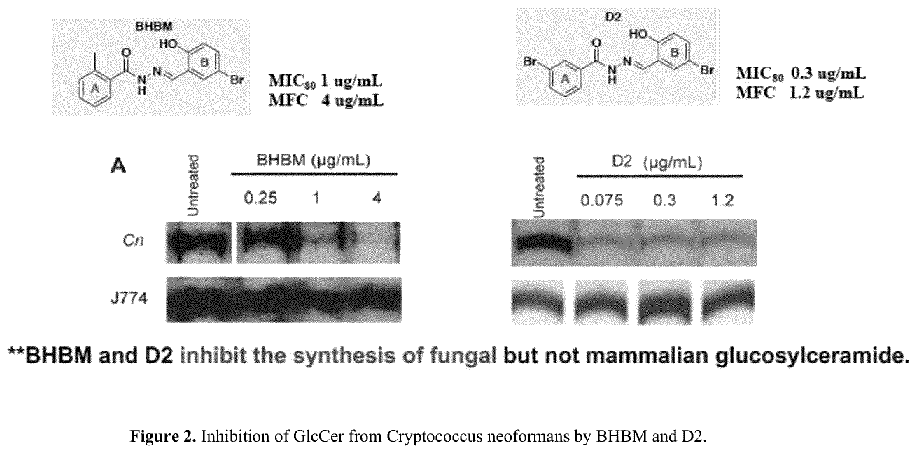

It was known that acylhydrazone analogs BHBM and compound D2 displayed potent antifungal activities by inhibiting the synthesis of sphingolipid GlcCer in C. neoformans (FIG. 2). Based on the structure of BHBM and D2, extensive SAR studies were carried out by synthesizing a library of ˜300 N-(aromatic acyl)-2-hydroxyarylhydrazone, which led the identification of several lead compounds that displayed excellent MIC₈₀, in vitro killing activity, and high selectivity indices. However, most of these novel acylhydrazones have poor solubility. Ethynyl functional group was introduced as the bioisosteric replacement of bromine moiety of the lead acylhydrazone, which resulted broad-spectrum in vitro evaluation of novel acylhydrazones bearing ethynyl phenyl as ring B.

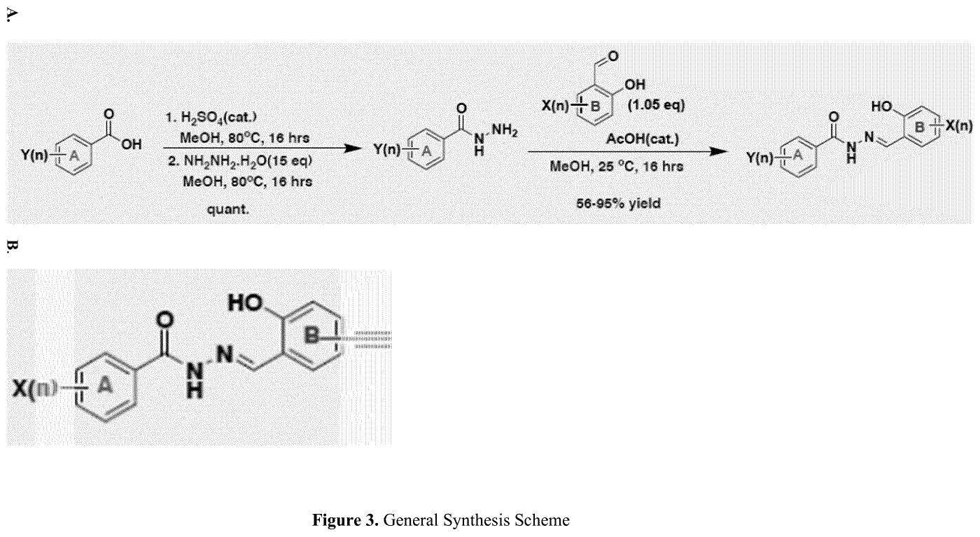

As shown in FIG. 3, commercially available substituted aromatic benzoic acids were converted to respective methyl esters, followed by treatment with hydrazine monohydrate to yield hydrazides. In some cases, commercially available substituted aromatic benzoic esters were directly treated with hydrazine monohydrate to yield hydrazides. The hydrazides were then coupled with a variety of salicylaldehydes to give corresponding acylhydrazones. Ethynyl group was incorporated on ring B with various bromo salicylaldehyde via sonogashira coupling.

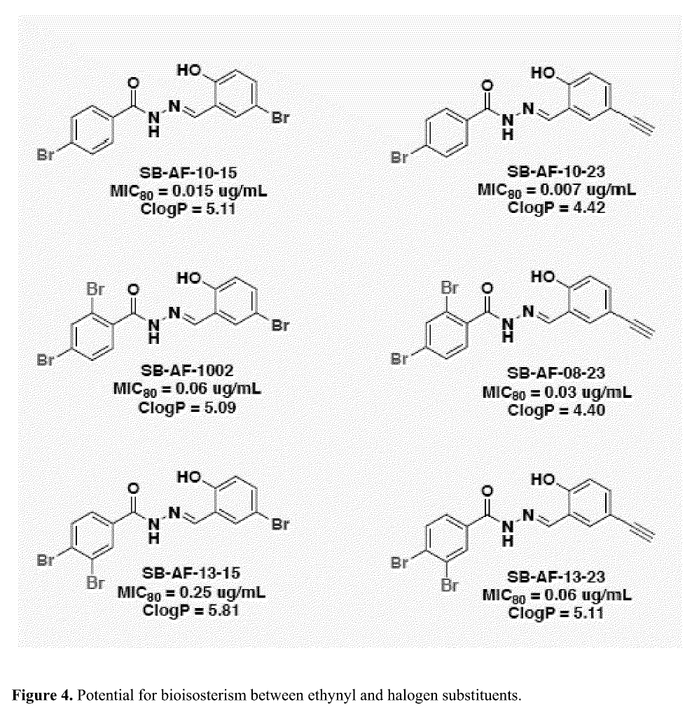

According to FIG. 4, the ethynyl group (or “acetylene CH”) group is an interesting example of a non-classical bioisostere of a non-classical bioisostere of halogens because of its versatility: its π cloud is useful for mimicking aromatic systems. Its polarized —CH moiety is a weak hydrogen bond donor and is a replacement for a Bromine atom. The molecular electrostatic potentials for halobenzenes and phenylacetylene are remarkably similar, both bearing an area of positive charge at the tip of the C—X/H bond (where X═Cl, Br, I, or ethynyl) as well as an area of negative charge perpendicular to the C—XH Bond. Ethynyl moiety was a potential bioisostere replacement of the bromine moiety of the acylhydrazones. A series of ethynyl bearing aromatic acylhydrazones were synthesized and tested against C. neoformans; all displayed MIC₈₀≤1 μg/mL. There are four compounds which possess broad-spectrum antifungal activity. SB-AF-08-27 displayed excellent in vitro broad-spectrum activity.

### REFERENCES

Aerts A M, et al. The antifungal activity of RsAFP2, a plant defensin from raphanus sativus, involves the induction of reactive oxygen species in Candida albicans. J Mol Microbiol Biotechnol. 2007; 13(4):243-7.
Beyer, C., and Wagenknecht, H.-A. (2010) Synthesis of Spiropyrans As Building Blocks for Molecular Switches and Dyads. J. Org. Chem., 75 (8), 2752-2755.
Bonifaz A, Tirado Sánchez A. 2017. Cutaneous disseminated and extracutaneous sporotrichosis: current status of a complex disease. J Fungi 3:6. 10.3390/jof3010006
Bligh E G, and Dyer W J. A rapid method for total lipid extraction and purification. Can J Bioch Physiol. 1959; 37; 911-7.
Artunduaga Bonilla J J, Honorato L, Haranahalli K., Gremião IDF, Pereira S A, Guimarães A, Baptista A R S, de M Tavares P, Rodrigues M L, Miranda K, Ojima I, Del Poeta M, Nimrichter L. Antifungal activity of Acylhydrazone derivatives against Sporothrix spp. Antimicrob Agents Chemother. 2021 Feb. 16; 65 (5):e02593-20. doi: 10.1128/AAC.02593-20. Epub ahead of print. PMID: 33593845; PMCID: PMC8092869.
Brilhante R S N, Rodrigues A M, Sidrim J J C, Rocha M F G, Pereira S A., Gremiaõ IDF, Schubach T M P, de Camargo Z P. 2016. In vitro susceptibility of antifungal drugs against Sporothrix brasiliensis recovered from cats with sporotrichosis in Brazil. Med Mycol 54:275-279. 10.1093/nrmmynvv039.
Brown G D, Denning D W, Gow N A, Levitz S M, Netea M G, and White T C. Hidden killers: human fungal infections. Sci Transl Med. 2012; 4(165):165rv13.
Carmona E M, and Limper A H. Update on the diagnosis and treatment of Pneumocystis pneumonia. Ther Adv Respir Dis. 2011; 5(1):41-59.
Chamilos G, Lewis R E, and Kontoyiannis D P. Lovastatin has significant activity against zygomycetes and interacts synergistically with voriconazole. Antimicrob Agents Chemother. 2006; 50(1):96-103.
Chung N, Mao C, Heitman J, Hannun Y A, and Obeid L M. Phytosphingosine as a specific inhibitor of growth and nutrient import in Saccharomyces cerevisiae. J Biol Chem. 2001; 276(38):35614-21.
Chung N, and Obeid L M. Use of yeast as a model system for studies of sphingolipid metabolism and signaling. Methods Enzymol. 2000; 311(8):319-31.
da Silva A F, et al. Glucosylceramides in Colletotrichum gloeosporioides are involved in the differentiation of conidia into mycelial cells. FEBS Lett. 2004; 561 (1-3):137-43.
DePristo, M. A., et al. A framework for variation discovery and genotyping using next-generation DNA sequencing data. Nature genetics 43, 491-498 (2011).
Del Poeta M, Nimrichter L, Rodrigues M L, and Luberto C. Synthesis and biological properties of fungal glucosylceramide. PLoS Pathog. 2014; 10 (1):e1003832.
Del Poeta M, et al. Synergistic antifungal activities of bafilomycin A(1), fluconazole, and the pneumocandin MK-0991/Caspofungin acetate (L-743,873) with calcineurin inhibitors FK506 and L-685,818 against Cryptococcus neoformans. Antimicrob Agents Chemother. 2000; 44(3):739-46.
Farowski F, et al. Intracellular concentrations of micafungin in different cellular compartments of the peripheral blood. Int J Antimicrob Agents. 2012; 39(3):228-31.
Farowski F, et al. Intracellular concentrations of anidulafungin in different compartments of the peripheral blood. Int J Antimicrob Agents. 2013; 41(4):379-82.
Funato K, and Riezman H. Vesicular and nonvesicular transport of ceramide from ER to the Golgi apparatus in yeast. J Cell Biol. 2001; 155(6):949-59.
Fungal Infection Trust, How common are fungal diseases? Fungal Research Trust 20th Anniversary Meeting. Fungal Infection Trust; London. Jun. 18th 2011, updated December 2012.
Guery B P, et al. Management of invasive candidiasis and candidemia in adult non-neutropenic intensive care unit patients: Part I. Epidemiology and diagnosis. Intensive Care Med. 2009; 35(1):55-62.
Gullo A. Invasive fungal infections: the challenge continues. Drugs. 2009; 69 Suppl 1,65-73.
Haranahalli K, Lazzarini C, Sun Y, Zambito J, Pathiranage S, McCarthy J B, Mallamo J, Del Poeta M, Ojima I. SAR Studies on Aromatic Acylhydrazone-Based Inhibitors of Fungal Sphingolipid Synthesis as Next-Generation Antifungal Agents. J Med Chem. 2019 Sep. 12; 62(17):8249-8273. doi: 10.1021/acs.imedchem.9b01004. Epub 2019 Aug. 16. PMID: 31369263; PMCID: PMC6755904.
Heung L J, Luberto C, and Del Poeta M. Role of sphingolipids in microbial pathogenesis. Infect Immun. 2006; 74(1):28-39.
Heung, L. J., Kaiser, A. E., Luberto, C. & Del Poeta, M. The role and mechanism of diacylglycerol-protein kinase C₁ signaling in melanogenesis by Cryptococcus neoformans. J. Biol. Chem. 280, 28547-28555 (2005).
Hoffman. C. S., Winston, F. A ten-minute DNA preparation from yeast efficiently releases autonomous plasmids for transformation of Escherichia coli. Gene 57:267-272 (1987).
Hua, Z. & Graham, T. R. Requirement for neolp in retrograde transport from the Golgi complex to the endoplasmic reticulum. Molecular biology of the cell 14, 4971-4983 (2003).
Huang L, Morris A, Limper A H, Beck J M, and Participants ATSPW. An Official ATS Workshop Summary: Recent advances and future directions in pneumocystis pneumonia (PCP). Proc Am Thorac Soc. 2006; 3(8):655-64.
Huang Z, et al. A functional variomics tool for discovering drug-resistance genes and drug targets. Cell Rep. 2013; 3(2):577-85.
Huang Z, et al. Sampangine inhibits heme biosynthesis in both yeast and human. Eukaryot Cell. 2011; 10(11):1536-44.
Hsu, D.-S., and Liou, C.-Y. (2018) Total synthesis and structural revision of (±)-nidemone. Org. Biomol. Chem., 16 (27), 4990-4995.
Kajiwara K, et al. Osh proteins regulate COPII-mediated vesicular transport of ceramide from the endoplasmic reticulum in budding yeast. J Cell Sci. 2014; 127 (Pt 2):376-87.
Kazanjian P, et al. Pneumocystis carinii cytochrome b mutations are associated with atovaquone exposure in patients with AIDS. J Infect Dis. 2001; 183(5):819-22.
Kechichian T B, et al. Depletion of alveolar macrophages decreases the dissemination of a glucosylceramide-deficient mutant of Cryptococcus neoformans in immunodeficient mice. Infect Immun. 2007; 75(10):4792-8.
Kelley C F, et al. Trends in hospitalizations for AIDS-associated Pneumocystis jirovecii Pneumonia in the United States (1986 to 2005). Chest. 2009; 136(1):190-197.
Lazzarini C, Haranahalli K, Rieger R. Ananthula H K, Desai P B, Ashbaugh A, Linke M J, Cushion M T, Ruzsicska B. Haley J, Ojima I, Del Poeta M. 2018. Acylhydrazones as antifungal agents targeting the synthesis of fingal sphingolipids. Antinicrob Agents Chenothcr 62:1-14, 10.1128/AAC.00156-18.
Lee A Y, et al. Mapping the cellular response to small molecules using chemogenomic fitness signatures. Science. 2014; 344(6180):208-11.
Levery S B, et al. Disruption of the glucosylceramide biosynthetic pathway in Aspergillus nidulans and Aspergillus fumigatus by inhibitors of UDP-Glc:ceramide glucosyltransferase strongly affects spore germination, cell cycle, and hyphal growth. FEBS Lett. 2002; 525 (1-3):59-64.
Li, R, et al. SOAP2: an improved ultrafast tool for short read alignment. BioInformatics 25:1966-1967 (2009).
Li, H. & Durbin, R. Fast and accurate short read alignment with Burrows-Wheeler transform. Bioinformatics 25, 1754-1760 (2009).
Lobo D S, et al. Antifungal Pisum sativum defensin 1 interacts with Neurospora crassa cyclin F related to the cell cycle. Biochemistry. 2007; 46(4):987-96.
Ma L, Borio L, Masur H, and Kovacs J A. Pneumocystis carinii dihydropteroate synthase but not dihydrofolate reductase gene mutations correlate with prior trimethoprim-sulfamethoxazole or dapsone use. J Infect Dis. 1999; 180(6):1969-78.
Mandala S M, et al. The discovery of australifungin, a novel inhibitor of sphinganine N-acyltransferase from Sporormiella australis. Producing organism, fermentation, isolation, and biological activity. J Antibiot (Tokyo). 1997; 50(4):339-43.
Mayr A, and Lass-Florl C. Epidemiology and antifungal resistance in invasive Aspergillosis according to primary disease: review of the literature. Eur J Med Res. 2011; 16(4):153-7.
McKenna, A., et al. The Genome Analysis Toolkit: a MapReduce framework for analyzing next-generation DNA sequencing data. Genome research 20, 1297-1303 (2010).
Mello Ede, O., et al. Functional expression and activity of the recombinant antifungal defensin PvD1r from Phaseolus vulgaris L. (common bean) seeds. BMC Biochem. 2014; 15(1):7.
Mor V, Rella A, Farnoud A M, Singh A, Munshi M, Bryan A, Naseem S, Konopka J B, Ojima I, Bullesbach E, Ashbaugh A, Linke M J, Cushion M, Collins M, Ananthula H K, Sallans L, Desai P B, Wiederhold N P, Fothergill A W, Kirkpatrick W R, Patterson T, Wong L H, Sinha S, Giaever G, Nislow C, Flaherty P, Pan X, Cesar G V, de Melo Tavares P, Frases S, Miranda K, Rodrigues M L, Luberto C, Nimrichter L, Del Poeta M. Identification of a New Class of Antifungals Targeting the Synthesis of Fungal Sphingolipids. mBio. 2015 Jun. 23; 6 (3):e00647. doi: 10.1128/mBio.00647-15. Erratum in: MBio. 2018 Mar. 13; 9 (2): PMID: 26106079; PMCID: PMC4479701.
Mor V, Rella A, Farnoud A M, Singh A, Munshi M, Bryan A, Naseem S, Konopka J B, Ojima I, Bullesbach E, Ashbaugh A, Linke M J, Cushion M, Collins M, Ananthula H K, Sallans L, Desai P B, Wiederhold N P, Fothergill A W, Kirkpatrick W R, Patterson T, Wong L H, Sinha S, Giaever G, Nislow C, Flaherty P, Pan X, Cesar G V, de Melo Tavares P, Frases S, Miranda K, Rodrigues M L, Luberto C, Nimrichter L, Del Poeta M. Identification of a New Class of Antifungals Targeting the Synthesis of Fungal Sphingolipids. mBio. 2015 Jun. 23; 6 (3):e00647. doi: 10.1128/mBio.00647-15. Erratum in: MBio. 2018 Mar. 13; 9 (2): PMID: 26106079; PMCID: PMC4479701.
Munoz P, Guinea J, Narbona M T, and Bouza E. Treatment of invasive fungal infections in immunocompromised and transplant patients: AmBiLoad trial and other new data. Int J Antimicrob Agents. 2008; 32 Suppl 2: S125-31.
Noble S M, French S, Kohn L A, Chen V, and Johnson A D. Systematic screens of a Candida albicans homozygous deletion library decouple morphogenetic switching and pathogenicity. Nat Genet. 2010; 42(7):590-8.
Oura T, and Kajiwara S. Candida albicans sphingolipid C₉-methyltransferase is involved in hyphal elongation. Microbiology. 2010; 156 (Pt 4):1234-43.
Oura T, and Kajiwara S. Disruption of the sphingolipid Delta8-desaturase gene causes a delay in morphological changes in Candida albicans. Microbiology. 2008; 154 (Pt 12):3795-803.
Pagano R E, Sepanski M A, and Martin O C. Molecular trapping of a fluorescent ceramide analogue at the Golgi apparatus of fixed cells: interaction with endogenous lipids provides a trans-Golgi marker for both light and electron microscopy. J Cell Biol. 1989; 109(5):2067-79.
Perlroth J, Choi B, and Spellberg B. Nosocomial fungal infections: epidemiology, diagnosis, and treatment. Med Mycol. 2007; 45(4):321-46.
Rhome R, et al. Biosynthesis and immunogenicity of glucosylceramide in Cryptococcus neoformans and other human pathogens. Eukaryot Cell. 2007; 6(10):1715-26.
Rhome R, et al. Surface localization of glucosylceramide during Cryptococcus neoformans infection allows targeting as a potential antifungal. PLoS One. 2011; 6 (1):e15572.
Rueping M J, eInvasive candidiasis and candidemia: from current opinions to future perspectives. Expert Opin Investig Drugs. 2009; 18(6):735-48.
Ruping M J, Vehreschild J J, and Comely O A. Patients at high risk of invasive fungal infections: when and how to treat. Drugs. 2008; 68(14):1941-62.
Saribas Z, Yurdakul P, Cetin-Hazirolan G, and Arikan-Akdagli S. Influence of serum on in vitro susceptibility testing of echinocandins for Candida parapsilosis and Candida guilliermondii. Mycoses. 2012; 55(2):156-60.
Sekarpandi, S., Jammi, S., and Punniyamurthy, T. (2012) Fluorescent non-linear chiral polymer chemosensor bonded alternatively with 1,4-diethynyl-2,5-dioctyloxybenzene and (R,R)-salen for Zn2+recognition. Tetrahedron: Asymmetry, 23, 101-107.
Singh A, and Del Poeta M. Lipid signalling in pathogenic fungi. Cellular microbiology. 2011; 13(2):177-85.
Singh A, Na C, Silva L C, Prieto M, Futerman A H, Luberto C, and Del Poeta M. Membrane lipid topography controlled by sphingolipids regulates pathogenicity of Cryptococcus neoformans. Cellular Microbiology. 2012; 14(4):500-16.
Singh J, Rimek D, and Kappe R. In vitro susceptibility of 15 strains of zygomycetes to nine antifungal agents as determined by the NCCLS M38-A microdilution method. Mycoses. 2005; 48(4):246-50.
Sorrell T C, Chen S C-A, Phillips P, and Marr K A. In: Heitman J, Kozel T R, Kwon-Chung K J, Perfect J, and Casadevall A eds. Cryptococcus: from human pathogen to model yeast. Washington, DC: ASM; 2011:595-606.
Suzuki, Y., et al. Knocking out multigene redundancies via cycles of sexual assortment and fluorescence selection. Nature methods 8, 159-164 (2011).
Tavares P M, et al. In vitro activity of the antifungal plant defensin RsAFP2 against Candida isolates and its in vivo efficacy in prophylactic murine models of candidiasis. Antimicrob Agents Chemother. 2008; 52(12):4522-5.
Thevissen K, et al. The plant defensin RsAFP2 induces cell wall stress, septin mislocalization and accumulation of ceramides in Candida albicans. Mol Microbiol. 2012; 84(1):166-80.
Thevissen K, et al. Defensins from insects and plants interact with fungal glucosylceramides. J Biol Chem. 2004; 279(6):3900-5.
Toledo M S, et al. Characterization of cerebrosides from the thermally dimorphic mycopathogen Histoplasma capsulatum: expression of 2-hydroxy fatty N-acyl (E)-Delta(3)-unsaturation correlates with the yeast-mycelium phase transition. Glycobiology. 2001; 11(2):113-24.
Wesp, A., et al. End4p/Sla2p interacts with actin-associated proteins for endocytosis in Saccharomyces cerevisiae. Molecular biology of the cell 8, 2291-2306 (1997).
World Health Organization. World Malaria Report 2013—http://www.who.int/malaria/publications/world_malaria report_2013/en/. Accessed Dec. 11, 2013.
World Health Organization. Global Tuberculosis Report 2013—http://www.who.int/tb/publications/global report/en/. Accessed November 2013.

1. A compound having the structure: 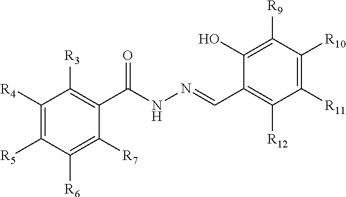
wherein
R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅;
R₉, R₁₀, R₁₁, and R₁₂ are each independently, H, —CN, alkenyl, alkynyl, aryl, heteroaryl, cycloalkyl, heterocycloalkyl, —OAc, —OR₁₃, —COR₁₃, —SH, —SR₁₃, —SO₂R₁₃, —NH₂, —NHR₁₃, —NR₁₄R₁₅, —NHCOR₁₂, or —CONR₁₄R₁₅;
wherein each occurrence of R₁₃ is independently alkyl, alkenyl, alkynyl, aryl, or heteroaryl,
wherein each occurrence of R₁₄ is independently —H, alkyl, alkenyl, alkynyl, aryl, or heteroaryl,
wherein each occurrence of R₁₅ is independently —H, alkyl, alkenyl, alkynyl, aryl, or heteroaryl,
wherein when R₁₄ is methyl, R₁₅ is not methyl;
wherein at least one of R₉, R₁₀, R₁₁, and R₁₂ is not H;
wherein at least one of R₃, R₄, R₅, R₆, and R₇ is not H;
wherein when one of R₉, R₁₀, R₁₁, and R₁₂ is —OR₁₃, R₃, R₄, R₅, R₆, and R₇ are not halogen or alkyl.

2. The compound of claim 1, wherein
(a) R₃, R₄, R₅, R₆, and R₇ are each independently, H, halogen, —CN, —CF₃, —OCF₃, —NO₂, alkyl, alkenyl, alkynyl, aryl, heteroaryl, heterocycle, —OH, —OAc, —OR₁₃, —COR₁₃, —CH₂OR₁₃, —SH, —SR₁₃, —SO₂R₁₃, —NH₂, —NHR₁₃, —NR₁₄R₁₅, —NHCOR₁₂, or —CONR₁₄R₁₅; and/or
(b) R₉, R₁₀, R₁₁, and R₁₂ are each independently, H, —CN, alkenyl, alkynyl, aryl, heteroaryl, cycloalkyl, heterocycloalkyl, —OAc, —COR₁₃, —SH, —SR₁₃, —SO₂R₁₃, —NH₂, —NHR₁₃, —NR₁₄R₁₅, —NHCOR₁₂, or —CONR₁₄R₁₅.

3. The compound of claim 1 having the structure: 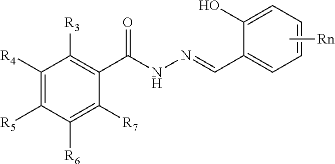
wherein
Rₙ is alkenyl, alkynyl, aryl, heteroaryl, cycloalkyl, or heterocycloalkyl.

4. The compound of claim 1, wherein R₃, R₄, R₅, R₆, and R₇ are each independently, H, halogen, —CN, alkyl, alkenyl, alkynyl, aryl, heteroaryl, heterocycle, —OCF₃, —OH, —OAc, —OR₁₃, —COR₁₃, —CH₂OR₁₃; wherein the heterocycle is six-membered heterocycle, five-membered heterocycle, four-membered heterocycle, or three-membered heterocycle.

5. (canceled)

6. The compound of claim 4 wherein
(a) R₃ is OH, R₄, R₅, R₆ and R₇ are each independently H or halogen;
(b) R₇ is OH, R₃, R₄, R₅ and R₆ are each independently H or halogen;
(c) R₃, R₄, R₅, R₆, R₇ are each independently, H or CH₂OR₁₃;
(d) R₃, R₄, R₅, R₆, and R₇ are each independently, H, halogen or —OCF₃;
(e) R₃, R₄, R₅, R₆, and R₇ are each H or heterocycle; or
(f) R₄, R₅, and R₆ are each H or heterocycle, R₇ is H.

7. The compound of claim 6, wherein [A]
a) R₇ is OH, R₄ and R₅ are halogen, R₃ and R₅ are H;
b) R₇ is OH, R₄ and R₆ are halogen, R₃ and R₅ are H;
c) R₇ is OH, R₃ and R₄ are halogen, R₅ and R₆ are H;
d) R₇ is OH, R₃ and R₅ are halogen, R₄ and R₆ are H;
e) R₇ is OH, R₃ and R₅ are halogen, R₄ and R₅ are H;
f) R₇ is OH, R₅ and R₆ are halogen, R₃ and R₄ are H;
g) R₇ is OH, R₃, R₄, and R₅ are H, R₆ is halogen;
h) R₇ is OH, R₃, R₄, and R₆ are H, R₅ is halogen;
i) R₇ is OH, R₄, R₅, and R₆ are H, R₃ is halogen; or
j) R₇ is OH, R₃, R₅, and R₆ are H, R₄ is halogen;
[B]
a) R₃ is OH, R₄, R₅, and R₆ are H, R₇ is halogen;
b) R₃ is OH, R₅, R₆, and R₇ are H, R₄ is halogen;
c) R₃ is OH, R₄, R₆, and R₇ are H, R₅ is halogen;
d) R₃ is OH, R₄, R₅, and R₇ are H, R₆ is halogen;
e) R₃ is OH, R₄ and R₅ are halogen, R₆ and R₇ are H;
f) R₃ is OH, R₄ and R₆ are halogen, R₅ and R₇ are H;
g) R₃ is OH, R₄ and R₇ are halogen, R₅ and R₆ are H;
h) R₃ is OH, R₅ and R₆ are halogen, R₄ and R₇ are H;
i) R₃ is OH, R₅ and R₇ are halogen, R₄ and R₆ are H; or
j) R₃ is OH, R₆ and R₇ are halogen, R₄ and R₅ are H
[C]
a) R₃ is CH₂OR₁₃, R₄, R₅, R₆, and R₇ are H;
b) R₄ is CH₂OR₁₃, R₃, R₅, R₆, and R₇ are H;
c) R₅ is CH₂OR₁₃, R₃, R₄, R₆, and R₇ are H;
d) R₆ is CH₂OR₁₃, R₃, R₄, R₅, and R₇ are H;
e) R₇ is CH₂OR₁₃, R₃, R₄, R₅, and R₆ are H;
f) R₃ and R₄ are CH₂OR₁₃, R₅, R₆, R₇ are H;
g) R₃ and R₅ are CH₂OR₁₃, R₄, R₆, R₇ are H;
h) R₃ and R₆ are CH₂OR₁₃, R₄, R₅, R₇ are H;
i) R₃ and R₇ are CH₂OR₁₃, R₄, R₅, R₆ are H;
j) R₄ and R₅ are CH₂OR₁₃, R₃, R₆, R₇ are H;
k) R₄ and R₆ are CH₂OR₁₃, R₃, R₅, R₇ are H;
l) R₄ and R₇ are CH₂OR₁₃, R₃, R₅, R₆ are H;
m) R₅ and R₆ are CH₂OR₁₃, R₃, R₄, R₇ are H;
n) R₅ and R₇ are CH₂OR₁₃, R₃, R₄, R₆ are H; or
o) R₆ and R₇ are CH₂OR₁₃, R₃, R₄, R₅ are H;
[D]
a) R₃ is halogen, R₄, R₅, and R₆ are H, R₇ is —OCF₃;
b) R₃ is halogen, R₅, R₆, and R₇ are H, R₄ is —OCF₃;
c) R₃ is halogen, R₄, R₆, and R₇ are H, R₅ is —OCF₃;
d) R₃ is halogen, R₄, R₅, and R₇ are H, R₆ is —OCF₃;
e) R₄ is halogen, R₃, R₅, and R₆ are H, R₇ is —OCF₃;
f) R₄ is halogen, R₃, R₅, and R₇ are H, R₆ is —OCF₃;
g) R₄ is halogen, R₃, R₆, and R₇ are H, R₅ is —OCF₃;
h) R₄ is halogen, R₅, R₆, and R₇ are H, R₃ is —OCF₃;
i) R₆ is halogen, R₃, R₄, and R₅ are H, R₇ is —OCF₃;
j) R₆ is halogen, R₃, R₄, and R₇ are H, R₅ is —OCF₃;
k) R₆ is halogen, R₅, R₆, and R₇ are H, R₃ is —OCF₃;
l) R₆ is halogen, R₃, R₅, and R₇ are H, R₄ is —OCF₃;
m) R₇ is halogen, R₃, R₄, and R₅ are H, R₆ is —OCF₃;
n) R₇ is halogen, R₃, R₄, and R₆ are H, R₅ is —OCF₃;
o) R₇ is halogen, R₄, R₅, and R₆ are H, R₃ is —OCF₃; or
p) R₇ is halogen, R₃, R₅, and R₆ are H, R₄ is —OCF₃;
[E]
a) R₃ is heterocycle, R₄, R₅, R₆, and R₇ are H;
b) R₄ is heterocycle, R₃, R₅, R₆, and R₇ are H;
c) R₅ is heterocycle, R₃, R₄, R₆, and R₇ are H;
d) R₆ is heterocycle, R₃, R₄, R₅, and R₇ are H; or
e) R₇ is heterocycle, R₃, R₄, R₅, and R₆ are H;
[F]
a) R₄ is heterocycle, R₃, R₅, R₆, and R₇ are H;
b) R₅ is heterocycle, R₃, R₄, R₆, and R₇ are H; or
c) R₆ is heterocycle, R₃, R₄, R₅, and R₇ are H.

8. The compound of claim 7, wherein the (a) halogen is F, Cl, Br, and I and/or (b) R₅ is CH₂OR₁₃ and R₁₃ is branched or unbranched alkyl.

9. (canceled)

10. The method of claim 8, wherein (a) R₁₃ is methyl, ethyl, propyl, or isopropyl; (b) the halogen is Br; and/or (c) the heterocycle is three-membered heterocycle and the three-membered heterocycle is 3-methyl-3-(trifluoromethyl)-3H-diazirine.

11. (canceled)

12. (canceled)

13. (canceled)

14. (canceled)

15. (canceled)

16. (canceled)

17. The compound of claim 1 having the structure: 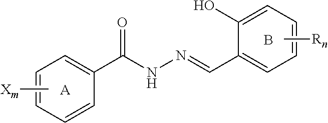
wherein ring A has the following structure: 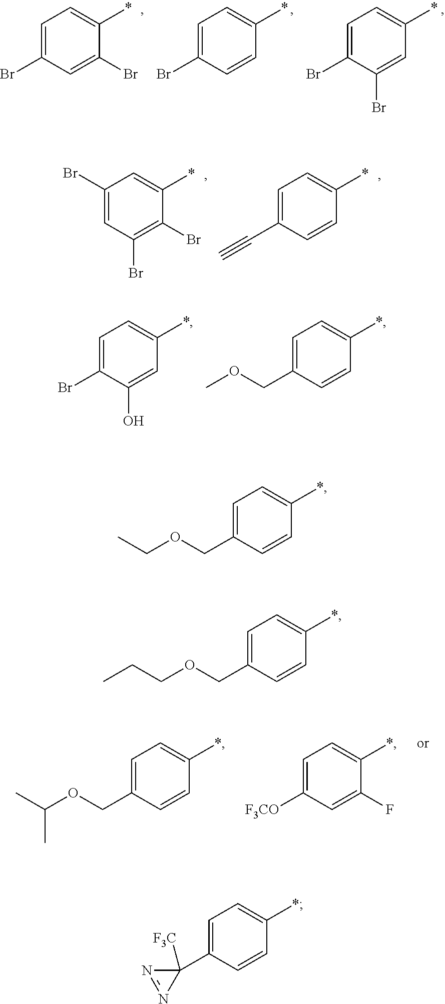
wherein ring B has the following structure:

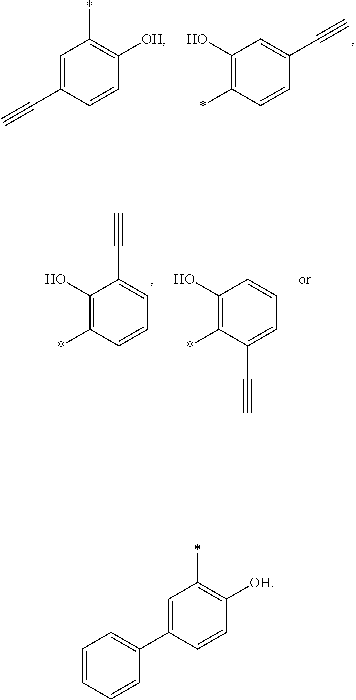

18. (canceled)

19. (canceled)

20. The compound of claim 1 having the structure: 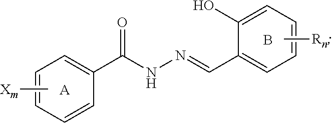
wherein Ring A has the following structure: 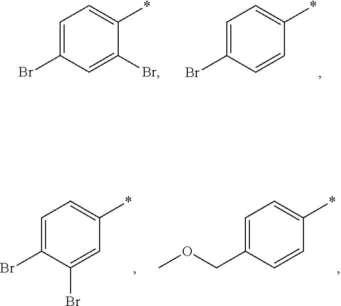
wherein Ring B has the following structure:

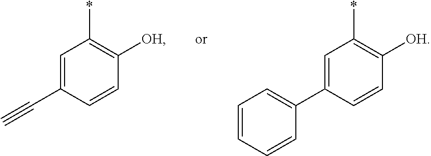

21. The compound of claim 1 having the structure:

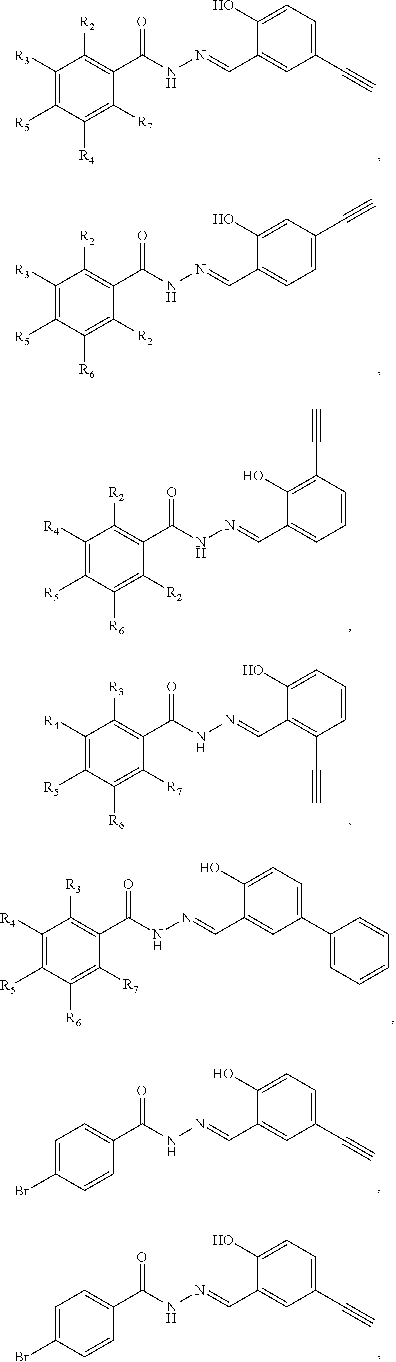

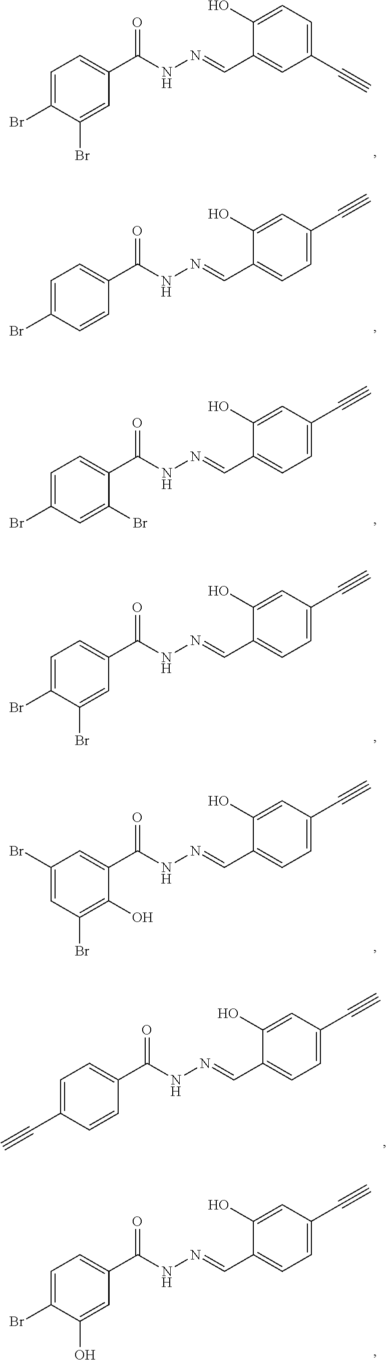

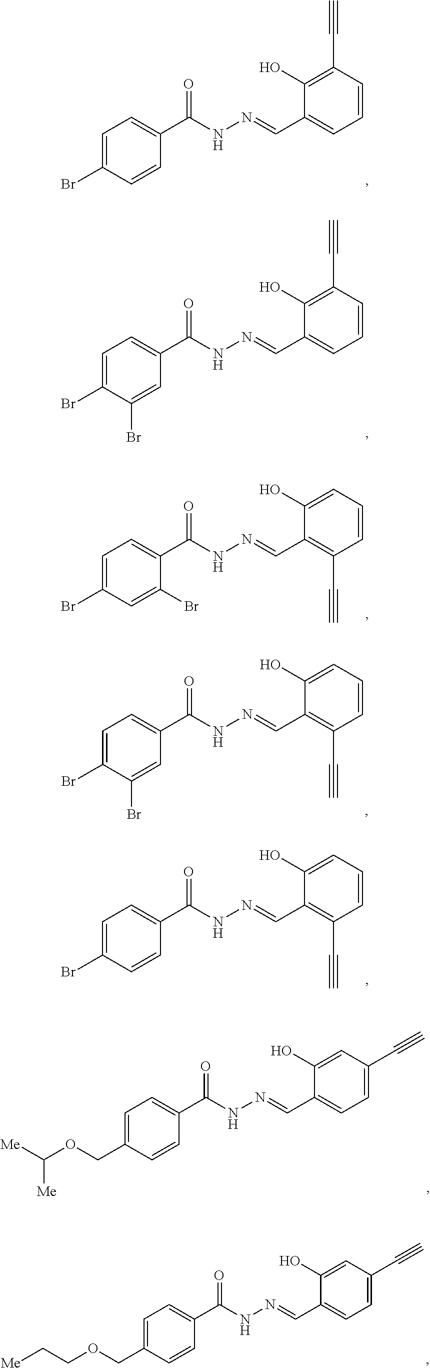

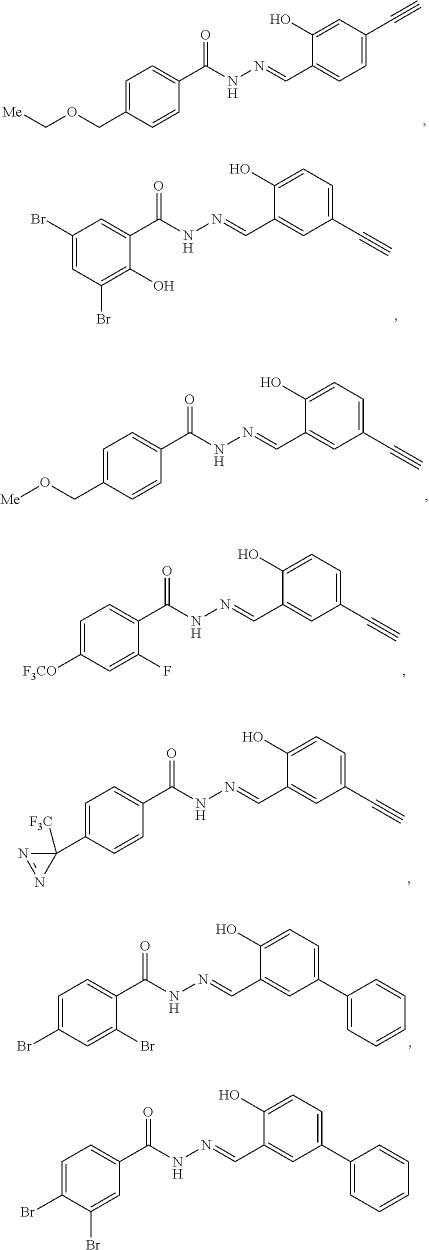

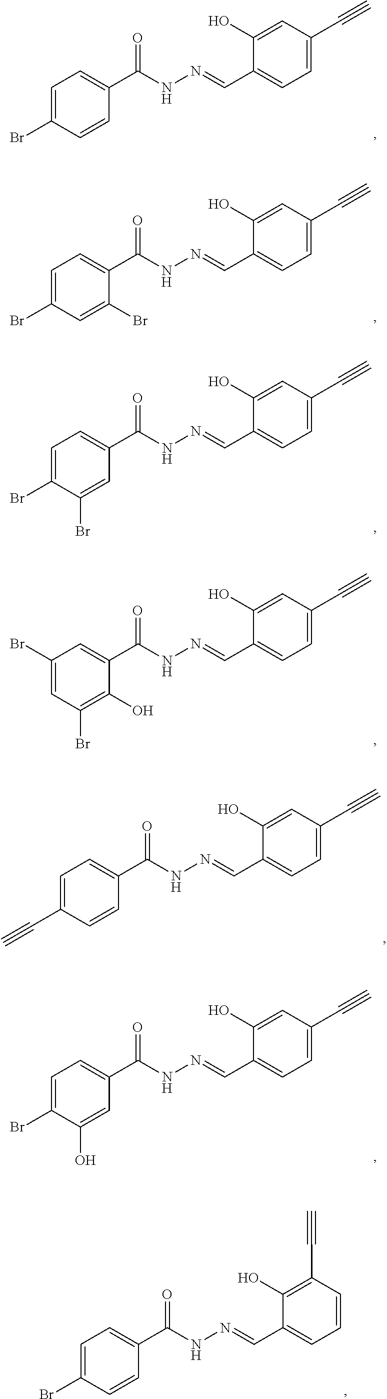

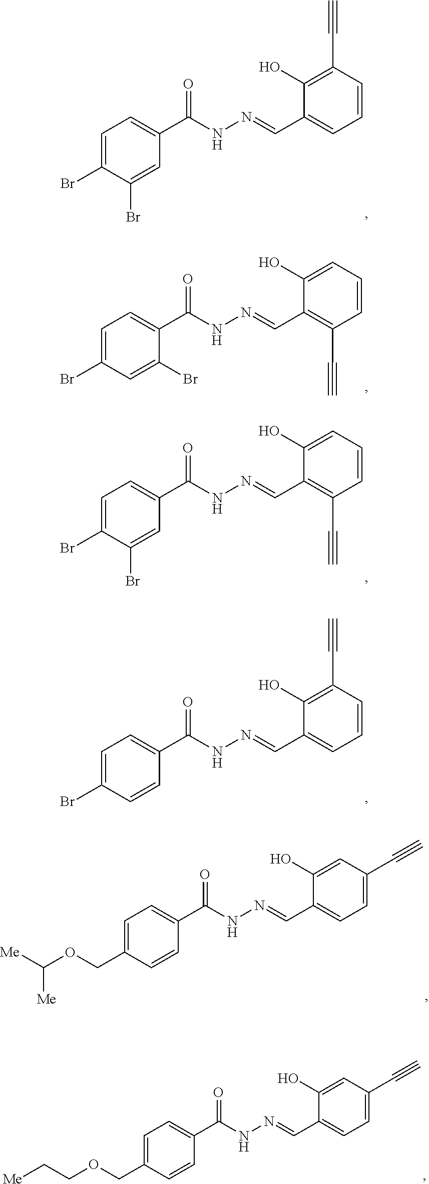

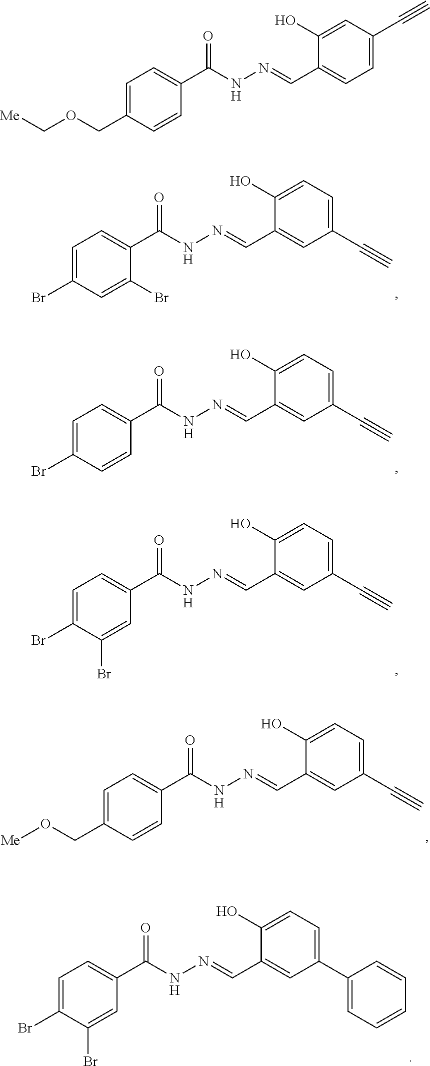 [SMILES: C#Cc1ccc(/C=N/NC(=O)c2ccc(COCC)cc2)c(O)c1 ; C#Cc1ccc(O)c(/C=N/NC(=O)c2ccc(Br)cc2Br)c1 ; C#Cc1ccc(O)c(/C=N/NC(=O)c2ccc(Br)cc2)c1 ; C#Cc1ccc(O)c(/C=N/NC(=O)c2ccc(Br)c(Br)c2)c1 ; C#Cc1ccc(O)c(/C=N/NC(=O)c2ccc(COC)cc2)c1 ; O=C(N/N=C/c1cc(-c2ccccc2)ccc1O)c1ccc(Br)c(Br)c1]

22. (canceled)

23. (canceled)

24. (canceled)

25. (canceled)

26. A compound having the structure: 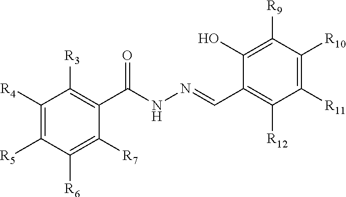
wherein
R₃, R₄, R₅, R₆, and R₇ are each independently H, alkenyl, alkynyl, aryl, heteroaryl, —OAc, —COR₁₃, —CH₂OR₁₃, —SH, —SR₁₃, —SO₂R₁₃, —NH₂, —NR₁₄R₁₅, —NHCOR₁₂, or —CONR₁₄R₁₅;
R₉, R₁₀, R₁₁, R₁₂ are each independently —H, halogen, —CN, —CF₃, —OCF₃, —NO₂, alkyl, alkenyl, alkynyl, aryl, heteroaryl, —OH, —OAc, —OR₁₃, —COR₁₃, —SH, —SR₁₃, —SO₂R₁₃, —SO₂NR₁₄R₁₅, —NH₂, —NHR₁₃, NR₁₄R₁₅, —NHCOR₁₂, or —CONR₁₄R₁₅;
wherein each occurrence of R₁₃ is independently alkyl, alkenyl, alkynyl, aryl, or heteroaryl;
wherein each occurrence of R₁₄ is independently —H, alkyl, alkenyl, alkynyl, aryl, or heteroaryl;
wherein each occurrence of R₁₅ is independently —H, alkyl, alkenyl, alkynyl, aryl, or heteroaryl;
wherein when R₁₄ is H, R₁₅ is not methyl or when R₁₅ is H, R₁₄ is not methyl;
wherein at least one of R₉, R₁₀, R₁₁, and R₁₂ is not H;
wherein at least one of R₃, R₄, R₅, R₆, and R₇ is not H.

27. The compound of claim 26, wherein R₃, R₄, R₅, R₆, and R₇ are each independently H, alkynyl, or —CH₂OR₁₃.

28. The compound of claim 27, wherein R₁₃ is alkyl; and/or wherein R₉, R₁₀, R₁₁, R₁₂ are each independently H, halogen, —CN, —CF₃, —OCF₃, —NO₂, alkyl, alkenyl, alkynyl, aryl, heteroaryl, —OH.

29. The compound of claim 28, wherein:
[A]
a) R₉ is halogen, R₁₀, R₁₁, and R₁₂ are H;
b) R₁₀ is halogen, R₉, R₁₁, and R₁₂ are H;
c) R₁₁ is halogen, R₉, R₁₀, and R₁₂ are H;
d) R₁₂ is halogen, R₉, R₁₀, and R₁₁ are H;
e) R₉ and R₁₀ are halogen, R₁₁ and R₁₂ are H;
f) R₉ and R₁₁ are halogen, R₁₀ and R₁₂ are H;
g) R₉ and R₁₂ are halogen, R₁₀ and R₁₁ are H;
h) R₁₀ and R₁₁ are halogen, R₉ and R₁₀ are H;
i) R₁₀ and R₁₂ are halogen, R₉ and R₁₁ are H; or
j) R₁₁ and R₁₂ are halogen, R₉ and R₁₀ are H;
[B]
a) R₃ is —CH₂OR₁₃, R₄, R₅, R₆, and R₇ are H;
b) R₄ is —CH₂OR₁₃, R₃, R₅, R₆, and R₇ are H;
c) R₅ is —CH₂OR₁₃, R₃, R₄, R₆, and R₇ are H;
d) R₆ is —CH₂OR₁₃, R₃, R₄, R₅, and R₇ are H;
e) R₇ is —CH₂OR₁₃, R₃, R₄, R₅, and R₆ are H;
f) R₃ and R₄ are —CH₂OR₁₃, R₅, R₆, R₇ are H;
g) R₃ and R₅ are —CH₂OR₁₃, R₄, R₆, R₇ are H;
h) R₃ and R₆ are —CH₂OR₁₃, R₄, R₅, R₇ are H;
i) R₃ and R₇ are —CH₂OR₁₃, R₄, R₅, R₆ are H;
j) R₄ and R₅ are —CH₂OR₁₃, R₃, R₆, R₇ are H;
k) R₄ and R₆ are —CH₂OR₁₃, R₃, R₅, R₇ are H;
l) R₄ and R₇ are —CH₂OR₁₃, R₃, R₅, R₆ are H;
m) R₅ and R₆ are —CH₂OR₁₃, R₃, R₄, R₇ are H;
n) R₅ and R₇ are —CH₂OR₁₃, R₃, R₄, R₆ are H; or
o) R₆ and R₇ are —CH₂OR₁₃, R₃, R₄, R₅ are H;
wherein the halogen is F, Br, Cl, or I.

30. (canceled)

31. The compound of claim 26 having the structure: 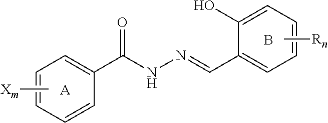
wherein Ring A has the following structure: 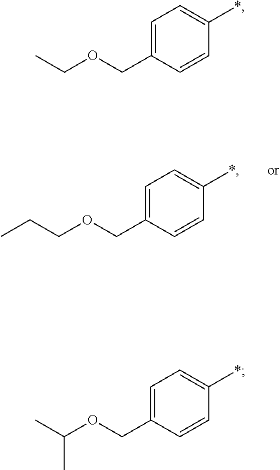
wherein Ring B has the following structure:

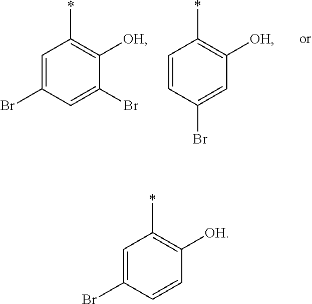

32. The compound of claim 31 having the structure:

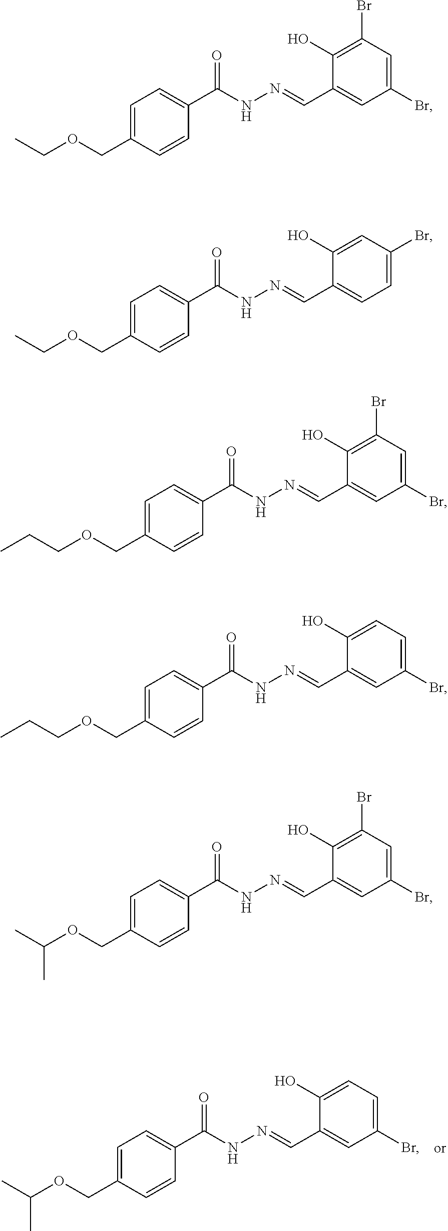 [SMILES: CCOCc1ccc(C(=O)N/N=C/c2cc(Br)cc(Br)c2O)cc1 ; CCOCc1ccc(C(=O)N/N=C/c2ccc(Br)cc2O)cc1 ; CCCOCc1ccc(C(=O)N/N=C/c2cc(Br)cc(Br)c2O)cc1 ; CCCOCc1ccc(C(=O)N/N=C/c2cc(Br)ccc2O)cc1 ; CC(C)OCc1ccc(C(=O)N/N=C/c2cc(Br)cc(Br)c2O)cc1 ; CC(C)OCc1ccc(C(=O)N/N=C/c2cc(Br)ccc2O)cc1]

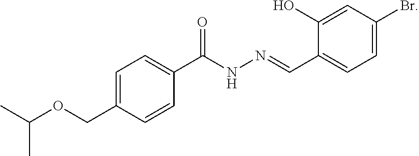 [SMILES: CC(C)OCc1ccc(C(=O)N/N=C/c2ccc(Br)cc2O)cc1]

33. A pharmaceutical composition comprising the compound of claim 1 and a pharmaceutically acceptable carrier.

34. A method of inhibiting the growth of a fungus comprising contacting the fungus with an effective amount of a compound of claim 1 or a pharmaceutically acceptable salt or ester thereof, so as to thereby inhibit the growth of the fungus; or a method of inhibiting fungal sphingolipid synthesis in a fungus comprising contacting the fungus with an effective amount of claim 1 or a pharmaceutically acceptable salt or ester thereof, so as to thereby inhibit sphingolipid synthesis in the fungus; or a method of inhibiting fungal sphingolipid synthesis in a fungus in a mammal without substantially inhibiting mammalian sphingolipid synthesis comprising administering to the mammal an effective amount of claim 1, or a pharmaceutically acceptable salt or ester thereof, so as to thereby inhibit fungal sphingolipid synthesis in the fungus in the mammal without substantially inhibiting mammalian sphingolipid synthesis.

35. (canceled)

36. (canceled)

37. (canceled)

38. (canceled)

39. The method of claim 34, further comprising contacting the fungus with an amount of an anti-fungal agent, wherein the anti-fungal agent is fluconazole, amphotericin B, caspofungin, tunicamycin or aureobasidin A; or wherein the fungus is Cryptococcus Neoformans, Cryptococcus gattii, Candida albicans, Candida krusei, Candida glabrata, Candida parapsilosis, Candida guilliermondii, Aspergillus fumigatus, Rhizopus oryzae, Rhizopus spp., Blastomyces dermatitis, Histoplasma capsulatum, Coccidioides spp., Paecilomyces variotii, Pneumocystis murina, Pneumocystis jiroveci, Histoplasma capsulatum, Aspergillus spp., S. brasiliensis, S. schenckii, S. globosa, S. mexicana, S. chilensis, S. luriei, or S. pallida.

40. (canceled)

41. (canceled)

42. (canceled)

43. (canceled)
